(19) United States
(12) Patent Application Publication
(10) Pub. No.: US 2024/0366726 A1
(43) Pub. Date: Nov. 7, 2024

(54) IL15/IL15R ALPHA HETERODIMERIC FC-FUSION PROTEINS FOR THE TREATMENT OF BLOOD CANCERS

(71) Applicants: Genentech, Inc., South San Francisco, CA (US); Xencor, Inc., Pasadena, CA (US)

(72) Inventor: Alexander Joachim Paul Ungewickell, South San Francisco, CA (US)

(21) Appl. No.: 18/423,941

(22) Filed: Jan. 26, 2024

Related U.S. Application Data
(63) Continuation of application No. PCT/US2022/074179, filed on Jul. 27, 2022.
(60) Provisional application No. 63/226,359, filed on Jul. 28, 2021.

(51) Int. Cl. A61K 38/20; A61K 38/17; A61P 37/04; C07K 16/28

(52) U.S. Cl. CPC A61K 38/2086; A61K 38/1793; A61P 37/04; C07K 16/2896

(57) ABSTRACT

The present disclosure provides methods of treating a blood cancer, such as multiple myelona, by administering a heterodimeric protein comprising a first monomer comprising an IL15 protein-Fc domain fusion and a second monomer comprising an IL15Rα protein-Fc domain fusion.

### CROSS-REFERENCE TO RELATED APPLICATIONS

This application claims priority to and benefit of U.S. Provisional Application No. 63/226,359, filed on Jul. 28, 2021, the contents of which are hereby incorporated by reference in their entirety.

### TECHNICAL FIELD

The present disclosure pertains to the field of treatment of blood cancer, such as multiple myeloma, using IL15-IL15R heterodimeric Fc-fusion proteins.

### SEQUENCE LISTING

The instant application contains a Sequence Listing which has been submitted electronically in XML format and is hereby incorporated by reference in its entirety. Said XML copy, created on Jul. 27, 2022, is named 000218-0046-WO1_SL.xml and is 60,483 bytes in size.

### BACKGROUND

Most blood cancers (or hematologic cancers) start in the bone marrow and result from abnormal blood cells growing out of control, interrupting the function of normal blood cells, which fight off infection and produce new blood cells. Multiple myeloma (MM), one type of blood cancer, is an incurable neoplasm characterized by the proliferation and accumulation of malignant plasma cells in the bone marrow that leads to the overproduction of monoclonal proteins (M-proteins) detectable in blood or urine of most subjects. A diagnosis of MM affects approximately 30,000 people every year in the United States (Siegel et a al. 2019), and approximately 160,000 people are diagnosed with MM annually worldwide (Bray et al. 2018). End-organ damage resulting from MM includes hypercalcemia, renal insufficiency, anemia, and lytic bone lesions. MM remains incurable despite advances in treatment, with an estimated median survival of 8-10 years for standard-risk and 2-3 years from high-risk myeloma, even with aggressive treatments such as autologous stem cell transplantation (ASCT) (Mikhael et al. 2013). Increased survival has been achieved with the introduction of proteasome inhibitors (PIs) such as bortezomib (Velcade® U.S. Package Insert [USPI]), immunomodulatory drugs (IMiDs) such as lenalidomide (Revlimid® USPI), and monoclonal antibodies such as daratumumab (Darzalex® USPI, Darzalex-Faspro™ USPI). Other agents with novel mechanisms of action that have received U.S. Food and Drug Administration approval for the treatment of MM include the nuclear export inhibitor Selinexor (Xpovio™ USPI) and belantamab mafodotin-blmf (Blenrep USPI).

Despite the significant progress in treatment options, most MM patients eventually relapse. Relapsed/refractory multiple myeloma (R/R MM) continues to constitute a significant unmet medical need, with median overall survival of less than one year in subjects who have disease refractory to anti-CD38 monoclonal antibodies (Chad et al. 2019; Ghandi et al. 2019). Several approaches that direct the human immune system to target and destroy malignant plasma cells are currently being explored in clinical settings, including T cell-engaging bispecific antibodies and chimeric antigen receptor [CAR] T cells. Emerging data from clinical studies using these agents suggest that manipulation of the subject's immune system is a potentially promising approach for the treatment of R/R MM (Moreau et al. 2019; Caraccio et al. 2020).

### SUMMARY

In a first aspect, the present disclosure provides a method of treating a blood cancer in a subject in need thereof, the method comprising administering to the subject a therapeutically effective amount of a heterodimeric protein, wherein the heterodimeric protein comprises (i) a first monomer comprising ILL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.

In a second aspect, the present disclosure provides a method for inducing the proliferation of CD8⁻ effector memory T cells in a subject suffering from a blood cancer, the method comprising administering to the subject an effective amount of a heterodimeric protein, wherein the heterodimeric protein comprises (i) a first monomer comprising IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.

In a third aspect, the present disclosure provides a method for inducing the proliferation of NK cells in a subject suffering from a blood cancer, the method comprising administering to the subject an effective amount of a heterodimeric protein, wherein the heterodimeric protein comprises (i) a first monomer comprising IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.

In a fourth aspect, the present disclosure provides a method for inducing the proliferation of CD8⁻ effector memory T cells and NK cells in a subject suffering from a blood cancer, the method comprising administering to the subject an effective amount of a heterodimeric protein, wherein the heterodimeric protein comprises (i) a first monomer comprising IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.

In a fifth aspect, the present disclosure provides a method for inducing IFNγ production in a subject suffering from a blood cancer, the method comprising administering to the subject an effective amount of a heterodimeric protein, wherein the heterodimeric protein comprises (i) a first monomer comprising IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.

In some embodiments, each of said first and second Fc domains comprises amino acid substitutions E233P, L234V, L235A, G236del, and S267K, according to EU numbering.

In some embodiments, the first Fc domain further comprises amino acid substitutions L368D and K370S and said second Fc domain further comprises amino acid substitutions S364K and E357Q, according to EU numbering. In some embodiments, the first Fc domain further comprises amino acid substitutions S364K and E357Q and said second Fc domain further comprises amino acid substitutions L368D and K370S, according to EU numbering. In some embodiments, the first Fc domain further comprises amino acid substitutions Q295E, N384D, Q418E and N421D, according to EU numbering.

In some embodiments, the second Fc domain further comprises amino acid substitutions Q295E, N384D, Q418E and N421D, according to EU numbering. In some embodiments, the second Fc domain further comprises amino acid substitution K246T, according to EU numbering.

In some embodiments, the IL-15 protein comprises amino acid substitutions D30N, E64Q and N65D. In some embodiments, the IL-15 protein comprises the amino acid sequence set forth in SEQ ID NO: 5.

In some embodiments, the sushi domain of IL-15Rα protein comprises the amino acid sequence set forth in SEQ ID NO: 4.

In some embodiments, the IL-15 protein is covalently attached to the N-terminus of the first Fc domain via a first linker.

In some embodiments, the IL-15Rα protein is covalently attached to the N-terminus of the second Fc domain via a second linker.

In some embodiments, the IL-15 protein is covalently attached to the N-terminus of the first Fc domain via a first linker and the IL-15Rα protein is covalently attached to the N-terminus of the second Fc domain via a second linker.

In some embodiments, the first linker and/or second linker is independently a variable length Gly-Ser linker. In some embodiments, the first linker and/or the second linker independently comprises a linker selected from the group consisting of (Gly-Gly-Gly-Gly-Ser)n (SEQ ID NO: 39), (Ser-Ser-Ser-Ser-Gly)n (SEQ ID NO: 40), (Gly-Ser-Ser-Gly-Gly)n (SEQ ID NO: 41), and (Gly-Gly-Ser-Gly-Gly)n (SEQ ID NO: 42), where n is an integer between 1 and 5.

In a sixth aspect, the present disclosure provides a method for treating a blood cancer in a subject in need thereof, the method comprising administering to the subject a therapeutically effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains comprises a set of amino acid substitutions selected from the group consisting of S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L; K370S: S364K/E357Q; S267K/S364K/E357Q: S267K/L368D/K370S; L368D/K370S: S364K/E357Q; S364K: L368D/K370S; S364K: L368E/K370S; D401K: T411E/K360E/Q362E; S364K/E357L: L368D/K370S; and S364K/E357Q: K370S, according to EU numbering.

In a seventh aspect, the present disclosure provides a method for inducing the proliferation of CD8⁺ effector memory T cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains comprises a set of amino acid substitutions selected from the group consisting of S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L; K370S: S364K/E357Q; S267K/S364K/E357Q: S267K/L368D/K370S; L368D/K370S: S364K/E357Q; S364K: L368D/K370S; S364K: L368E/K370S; D401K: T411E/K360E/Q362E; S364K/E357L: L368D/K370S; and S364K/E357Q: K370S, according to EU numbering.

In an eighth aspect, the present disclosure provides a method for inducing the proliferation of NK cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains comprises a set of amino acid substitutions selected from the group consisting of S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L; K370S: S364K/E357Q; S267K/S364K/E357Q: S267K/L368D/K370S; L368D/K370S: S364K/E357Q; S364K: L368D/K370S; S364K: L368E/K370S; D401K: T411E/K360E/Q362E; S364K/E357L: L368D/K370S; and S364K/E357Q: K370S, according to EU numbering.

In a ninth aspect, the present disclosure provides a method for inducing the proliferation of CD8⁻ effector memory T cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains comprises a set of amino acid substitutions selected from the group consisting of S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L; K370S: S364K/E357Q; S267K/S364K/E357Q: S267K/L368D/K370S; L368D/K370S: S364K/E357Q; S364K: L368D/K370S; S364K: L368E/K370S; D401K: T411E/K360E/Q362E; S364K/E357L: L368D/K370S; and S364K/E357Q: K370S, according to EU numbering.

In a tenth aspect, this present disclosure provides a method for inducing the proliferation of NK cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains comprises a set of amino acid substitutions selected from the group consisting of S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L; K370S: S364K/E357Q; S267K/S364K/E357Q: S267K/L368D/K370S; L368D/K370S: S364K/E357Q; S364K: L368D/K370S; S364K: L368E/K370S; D401K: T411E/K360E/Q362E; S364K/E357L: L368D/K370S; and S364K/E357Q: K370S, according to EU numbering.

In an eleventh aspect, this present disclosure provides a method for inducing the proliferation of CD8⁻ effector memory T cells and NK cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains comprises a set of amino acid substitutions selected from the group consisting of S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L; K370S: S364K/E357Q; S267K/S364K/E357Q: S267K/L368D/K370S; L368D/K370S: S364K/E357Q; S364K: L368D/K370S; S364K: L368E/K370S; D401K: T411E/K360E/Q362E; S364K/E357L: L368D/K370S; and S364K/E357Q: K370S, according to EU numbering.

In an twelfth aspect, this present disclosure provides a method for inducing IFNγ production in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains comprises a set of amino acid substitutions selected from the group consisting of S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L; K370S: S364K/E357Q; S267K/S364K/E357Q: S267K/L368D/K370S; L368D/K370S: S364K/E357Q; S364K: L368D/K370S; S364K: L368E/K370S; D401K: T411E/K360E/Q362E; S364K/E357L: L368D/K370S; and S364K/E357Q: K370S, according to EU numbering.

In some embodiments, the first and/or second Fc domains independently further comprises amino acid substitutions Q295E, N384D, Q418E and N421D, according to EU numbering.

In some embodiments, the first and/or second Fc domains independently further comprises amino acid substitutions selected from the group consisting of G236R/L328R; E233P/L234V/L235A/G236del/S239K; E233P/L234V/L235A/G236del/S267K; E233P/L234V/L235A/G236del/S239K/A327G; E233P/L234V/L235A/G236del/S267K/A327G; and E233P/L234V/L235A/G236del, according to EU numbering and wherein the Fc domains are derived from IgG1 or IgG3 Fc domains.

In some embodiments, the first and/or second Fc domains independently further comprises amino acid substitutions selected from the group consisting of L328R; S239K; and S267K, according to EU numbering and wherein the Fc domains are derived from IgG2 Fc domain.

In some embodiments, the first and/or second Fc domains independently further comprises amino acid substitutions selected from the group consisting of G236R/L328R; E233P/F234V/L235A/G236del/S239K; E233P/F234V/L235A/G236del/S267K; E233P/F234V/L235A/G236del/S239K/A327G; E233P/F234V/L235A/G236del/S267K/A327G; and E233P/F234V/L235A/G236del, according to EU numbering and wherein the Fc domains are derived from IgG4 Fc domain.

In some embodiments, the IL-15 protein comprises one or more amino acid substitutions selected from the group consisting of N1D, N4D, D8N, D30N, D61N, E64Q, N65D and Q108E.

In some embodiments, the IL-15 protein and said IL-15Rα protein comprise a set of amino acid substitutions or additions selected from E87C: 65DPC; E87C: 65DCA; V49C: S40C; L52C: S40C; E89C: K34C; Q48C: G38C; E53C: L42C; C42S: A37C and L45C: A37C, respectively.

In some embodiments, the IL-15 protein comprises a polypeptide sequence selected from the group consisting of SEQ ID NO:1 and SEQ ID NO:2.

In some embodiments, the IL-15Rα protein comprises a polypeptide sequence selected from the group consisting of SEQ ID NO:3 and SEQ ID NO:4.

In some embodiments, the first Fc domain comprises amino acid substitutions L368D and K370S; wherein the second Fc domain further comprises amino acid substitutions S364K and E357Q; and wherein each of said first and second Fc domains further comprises amino acid substitutions C220S, E233P, L234V, L235A, G236del, S267K, M428L and N434S, according to EU numbering; wherein said IL-15 protein comprises amino acid substitutions D30N, E64Q and N65D; and wherein said IL-15Rα protein comprises SEQ ID NO:4.

In some embodiments, the first Fc domain comprises amino acid substitutions S364K and E357Q; wherein the second Fc domain comprises amino acid substitutions L368D and K370S; and wherein each of said first and second Fc domains further comprises amino acid substitutions C220S, E233P, L234V, L235A, G236del, S267K, M428L and N434S, according to EU numbering; wherein said IL-15 protein comprises amino acid substitutions D30N, E64Q and N65D; and wherein said IL-15Rα protein comprises SEQ ID NO:4.

In some embodiments, the first Fc domain comprises amino acid substitutions L368D and K370S; wherein the second Fc domain comprises amino acid substitutions K246T, S364K and E357Q; and wherein each of said first and second Fc domains further comprises amino acid substitutions C220S, E233P, L234V, L235A, G236del, S267K, M428L and N434S, according to EU numbering; wherein said IL-15 protein comprises amino acid substitutions D30N, E64Q and N65D; and wherein said IL-15Rα protein comprises SEQ ID NO:4.

In some embodiments, the first Fc domain comprises amino acid substitutions S364K and E357Q; wherein the second Fc domain comprises amino acid substitutions K246T, L368D and K370S; and wherein each of said first and second Fc domains further comprises amino acid substitutions C220S, E233P, L234V, L235A, G236del, S267K, M428L and N434S, according to EU numbering; wherein said IL-15 protein comprises amino acid substitutions D30N, E64Q and N65D; and wherein said IL-15Rα protein comprises SEQ ID NO:4.

In some embodiments, the IL-15 protein is covalently attached to the N-terminus of the first Fc domain via a first linker. In some embodiments, the IL-15Rα protein is covalently attached to the N-terminus of the second Fc domain via a second linker. In some embodiments, the IL-15 protein is covalently attached to the N-terminus of the first Fc domain via a first linker and the IL-15Rα protein is covalently attached to the N-terminus of the second Fc domain via a second linker.

In some embodiments, the first linker and/or second linker is independently a variable length Gly-Ser linker. In some embodiments, the second linker independently comprises a linker selected from the group consisting of (Gly-Gly-Gly-Gly-Ser)n (SEQ ID NO: 39), (Ser-Ser-Ser-Ser-Gly)n (SEQ ID NO: 40), (Gly-Ser-Ser-Gly-Gly)n (SEQ ID NO: 41), and (Gly-Gly-Ser-Gly-Gly)n (SEQ ID NO: 42), where n is an integer between 1 and 5.

In some embodiments, the heterodimeric protein is selected from the group consisting of XENP22822, XENP23504, XENP24045, XENP24306, XENP22821, XENP23343, XENP23557, XENP24113, XENP24051, XENP24341, XENP24052, XENP24301, and XENP32803 proteins.

In some embodiments, the first monomer comprises the amino acid sequence set forth in SEQ ID NO: 9, and the second monomer comprises the amino acid sequence set forth in SEQ ID NO: 10. In some embodiments, the first monomer comprises the amino acid sequence set forth in SEQ ID NO: 9, and the second monomer comprises the amino acid sequence set forth in SEQ ID NO: 16.

In some embodiments, the heterodimeric protein is XENP24306, XENP32803, or a combination thereof.

In some embodiments, a combination of a first heterodimeric protein and a second heterodimeric protein is administered to the subject. In some embodiments, the first heterodimeric protein comprises a first monomer comprising the amino acid sequence set forth in SEQ ID NO: 9, and a second monomer comprising the amino acid sequence set forth in SEQ ID NO: 10; and the second heterodimeric protein comprises a first monomer comprising the amino acid sequence set forth in SEQ ID NO: 9, and a second monomer comprising the amino acid sequence set forth in SEQ ID NO: 16.

In some embodiments, the first and second heterodimeric proteins are administered simultaneously. In some embodiments, the first and second heterodimeric proteins are administered sequentially.

In some embodiments, the blood cancer is selected from the group consisting of leukemia, acute myeloid leukemia, adult acute lymphoblastic leukemia, chronic lymphocytic leukemia, lymphoma, non-Hodgkin's lymphoma, B-cell non-Hodgkin's lymphoma, and multiple myeloma.

In some embodiments, the blood cancer is multiple myeloma. In some embodiments, the multiple myeloma is relapsed or refractory multiple myeloma.

In some embodiments, the blood cancer is B-cell non-Hodgkin's lymphoma.

In some embodiments, the blood cancer is chronic lymphocytic leukemia.

In some embodiments, the subject has been previously administered one or more prior treatments. In some embodiments, the prior treatment is an immunomodulatory drug, a proteasome inhibitor, or an anti-CD38 monoclonal antibody. In some embodiments, the immunomodulatory drug is selected from the group consisting of lenalidomide, thalidomide, and pomalidomide. In some embodiments, the proteasome inhibitor is selected from the group consisting of bortezomib, carfilzomib, and ixazomib. In some embodiments, the anti-CD38 monoclonal antibody is selected from the group consisting of daratumumab, isatuximab, mezagitamab, and felzartamab.

In some embodiments, the heterodimeric protein or combination of heterodimeric proteins is administered at a dose selected from the group consisting of about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.1 mg/kg, about 0.12 mg/kg, about 0.16 mg/kg, about 0.2 mg/kg, about 0.24 mg/kg and about 0.32 mg/kg body weight.

In some embodiments, the heterodimeric protein or combination of heterodimeric proteins is administered at a dose selected from the group consisting of about 0.01 mg/kg, about 0.02 mg/kg, about 0.04 mg/kg, and about 0.06 mg/kg body weight.

In some embodiments, the heterodimeric protein or combination of heterodimeric proteins is administered at a dose selected from the group consisting of 0.0025 mg/kg, 0.005 mg/kg, 0.01 mg/kg, 0.015 mg/kg, 0.02 mg/kg, 0.025 mg/kg, 0.03 mg/kg, 0.04 mg/kg, 0.05 mg/kg, 0.06 mg/kg, 0.08 mg/kg, 0.10 mg/kg, 0.16 mg/kg, 0.20 mg/kg, 0.24 mg/kg and 0.32 mg/kg body weight.

In some embodiments, the heterodimeric protein or combination of heterodimeric proteins is administered at a dose selected from the group consisting of 0.01 mg/kg, 0.02 mg/kg, 0.04 mg/kg, and 0.06 mg/kg body weight.

In some embodiments, the method further comprises administering to the subject an anti-CD38 monoclonal antibody.

In some embodiments, the anti-CD38 monoclonal antibody is selected from the group consisting of daratumumab, isatuximab, mezagitamab, and felzartamab.

In some embodiments, the anti-CD38 monoclonal antibody is daratumumab.

In some embodiments, the heterodimeric protein and said anti-CD38 monoclonal antibody are administered simultaneously. In some embodiments, the heterodimeric protein and said anti-CD38 monoclonal antibody are administered sequentially.

In some embodiments, the heterodimeric protein is administered at a frequency selected from the group consisting of Q1W, Q2W, Q3W, Q4W, Q5W and Q6W. In some embodiments, the heterodimeric protein is administered at a frequency of Q1W in one or more cycles. In some embodiments, the heterodimeric protein is administered at a frequency of Q2W in one or more cycles. In some embodiments, the heterodimeric protein is administered at a frequency of Q4W in one or more cycles.

In some embodiments, the anti-CD38 monoclonal antibody is administered at a frequency selected from the group consisting of Q1W, Q2W, Q3W, Q4W, Q5W and Q6W. In some embodiments, the anti-CD38 monoclonal antibody is administered at a frequency of Q1W in one or more cycles. In some embodiments, the anti-CD38 monoclonal antibody is administered at a frequency of Q2W in one or more cycles. In some embodiments, the anti-CD38 monoclonal antibody is administered at a frequency of Q4W in one or more cycles.

In some embodiments, the heterodimeric protein is administered at a frequency of Q2W, and wherein said anti-CD38 monoclonal antibody is administered at a frequency of Q1W in one or more cycles. In some embodiments, the heterodimeric protein is administered at a frequency of Q2W, and wherein said anti-CD38 monoclonal antibody is administered at a frequency of Q2W in one or more cycles. In some embodiments, the heterodimeric protein is administered at a frequency of Q4W, and wherein said anti-CD38 monoclonal antibody is administered at a frequency of Q4W in one or more cycles.

In some embodiments, the heterodimeric protein is administered by intravenously.

In some embodiments, the anti-CD38 monoclonal antibody is administered subcutaneously.

### BRIEF DESCRIPTION OF THE DRAWINGS

FIGS. 1A and 1B show that a combination of XENP24306 (˜82%) and XENP32803 (˜18%) promotes dose-dependent proliferation of human NK cells (FIG. 1A) and CD8⁺ T cells (FIG. 1B) in human PBMCs. PBMC from 22 unique human donors were treated with indicated total concentrations of the combination of XENP24306 (˜82%) and XENP32803 (˜18%) for 4 days, and Ki67⁺ (marker of cell proliferation) frequency was determined by flow cytometry for CD3⁻ CD56⁺ NK cells (FIG. 1A) or CD3⁺CD8⁺CD16⁻ T cells (FIG. 1B). Each point represents the average value of 22 donors and error bars represent SEM. Curve fits were generated using the least squares method. EC₅₀ values were determined by non-linear regression analysis using agonist versus response using a variable-slope (four-parameter) equation. [CD=cluster of differentiation; NK=natural killer; PBMC=peripheral blood mononuclear cell].

FIG. 2 shows a comparison of CD8⁺ terminal effector T cell proliferation induced by a combination of XENP24306 (˜82%) and XENP32803 (˜18%), recombinant wild-type IL-15 (rIL15) and wild-type IL-15/wild-type IL-15Rα heterodimer Fc fusion (XENP22853) in human PBMCs. [EC₅₀=half maximal effective concentration].

FIGS. 3A-3D show graphs representing CD8β⁺ T cells (FIGS. 3A (males) and 3B (females)) and NK cells (FIGS. 3C (males) and 3D (females)) absolute count in whole blood of cynomolgus monkeys treated with repeat doses of a combination of XENP24306 (˜82%) and XENP32803 (˜18%) and different doses (0; 0.03 mg/kg; 0.2 mg/kg and 0.6 mg/kg). Whole blood from cynomolgus monkeys was stained with antibodies to identify CD8⁺ T cells as CD45⁺CD3⁺CD8β⁺CD4⁻ CD16⁻ and NK cells as CD45⁺ CD3⁻ CD16⁺. Each data point represents the mean of 3 to 5 cynomolgus monkeys per group; error bars denote SD.

FIG. 4 is a graph representing mean (±SD) heterodimeric protein (a combination of XENP24306 (˜82%) and XENP32803 (˜18%)) serum concentration (ng/mL) versus time (days) profiles in cynomolgus monkeys (males and females combined) following heterodimeric protein Q2W intravenous dosing (doses of 0.03 mg/kg; 0.2 mg/kg and 0.6 mg/kg) for a total of 3 doses.

FIG. 5 is the combination study schema for an IL15/IL15Rα heterodimeric protein (e.g., XENP24306, XENP32803, or a combination of XENP24306 (˜82%) and XENP32803 (˜18%)) and Daratumumab, showing subjects enrolled in two stages: a dose-escalation stage and an expansion stage and details on these two stages. DL=dose level; DLT=dose-limiting toxicity; MAb=monoclonal antibody; MAD=maximum administered dose; MTD=maximum tolerated dose; RP2D=recommended Phase II dose; SC=subcutaneous; TBD=to be determined. ^{a}Alternative IL15/IL15Rα dosing schedules may be considered based on accumulating safety data in this study. ^{b}See FIG. 6 for Daratumumab SC dosing schedule. ^{c}Safety threshold is defined as a DLT in one subject or a Grade ≥2 major organ adverse event not attributable to another clearly identifiable cause in at least 2 subjects during the DLT assessment window in a given cohort.

FIG. 6 is the combination therapy study schema for an IL15/IL15Rα heterodimeric protein (e.g., XENP24306, XENP32803, or a combination of XENP24306 (˜82%) and XENP32803 (˜18%) in combination with daratumumab (anti-CD38 antibody), showing a dosing schedule. SC=subcutaneous; IV=intravenous; TBD=to be determined; C=cycle; Q1W=every week; Q2W=every 2 weeks; Q4W=every 4 weeks; wk=weeks. Subjects may continue to receive treatment with IL15/IL15Rα until they meet criteria for study treatment discontinuation, discontinue the study, or the Sponsor terminates the study.

FIG. 7 provides the amino acid sequences for XENP24306 monomer 1 (SEQ ID NO: 9), XENP24306 monomer 2 (SEQ ID NO: 10), XENP32803 monomer 1 (SEQ ID NO: 9), and XENP32803 monomer 2 (SEQ ID NO: 16). In the monomer 1 sequences, the IL15 portion is underlined, the linker is offset with slashes and is bold and underlined, and the Fc portion follows the second slash and does not contain any formatting. In the monomer 2 sequences, the IL15Rα portion is underlined, the linker is offset with slashes and is bold and underlined, and the Fc portion follows the second slash and does not contain any formatting.

FIGS. 8A and 8B provides the amino acid sequences for the human IL-15 precursor protein (full-length human IL-15) (SEQ ID NO: 2), the mature or truncated human IL-15 protein (SEQ ID NO: 1), the full-length human IL-15Rα protein (SEQ ID NO: 3), the extracellular domain of the human IL-15Rα protein (SEQ ID NO: 54), the sushi domain of the human IL-15Rα protein (SEQ ID NO: 4), the full-length human IL-15Rβ protein (SEQ ID NO: 55) and the extracellular domain of the human IL-15Rβ protein (SEQ ID NO: 56).

FIGS. 9A to 9G provides the amino acid sequences for XENP22853 wild-type IL-15-Fc first monomer (SEQ ID NO: 11), XENP22822 protein (SEQ ID NO: 19 and SEQ ID NO: 20), XENP23504 protein (SEQ ID NO: 29 and SEQ ID NO: 30), XENP24045 protein (SEQ ID NO: 23 and SEQ ID NO: 24), XENP22821 protein (SEQ ID NO: 17 and SEQ ID NO: 18), XENP23343 protein (SEQ ID NO: 31 and SEQ ID NO: 32), XENP23557 protein (SEQ ID NO: 21 and SEQ ID NO: 22), XENP24113 protein (SEQ ID NO: 33 and SEQ ID NO: 34), XENP24051 protein (SEQ ID NO: 25 and SEQ ID NO: 26), XENP24341 protein (SEQ ID NO: 35 and SEQ ID NO: 36), XENP24052 protein (SEQ ID NO: 27 and SEQ ID NO: 28), and XENP24301 protein (SEQ ID NO: 37 and SEQ ID NO: 38).

### DETAILED DESCRIPTION

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Fourth edition, Cold Spring Harbor Laboratory Press, 2012; Ausubel et al., CURRENT PROTOCOLS IN MOLECULAR BIOLOGY, John Wiley & Sons, New York, 1987-2010 and periodic updates.

The term “herein” means the entire application.

It should be understood that any of the embodiments described herein, including those described under different aspects of the disclosure and different parts of the specification (including embodiments described only in the Examples) can be combined with one or more other embodiments disclosed herein, unless explicitly disclaimed or improper. Combination of embodiments are not limited to those specific combinations claimed via the multiple dependent claims.

Any publications, patents and published patent applications referred to in this application are specifically incorporated by reference herein. In case of conflict, the present specification, including its specific definitions, will control.

Throughout this specification, the word “comprise” or variations such as “comprises” or “comprising,” which is synonymous with “including,” “containing,” or “characterized by,” is inclusive or open-ended and does not exclude additional, unrecited elements or method steps.

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “consisting of” excludes any element, step, or ingredient not specifically recited.

The term “consisting essentially of” limits the scope of a disclosure to the specified materials or steps and those that do not materially affect the basic and novel characteristic(s) of the disclosure.

Any example(s) following the term “e.g.” or “for example” is not meant to be exhaustive or limiting.

The articles “a,” “an” and “the” are used herein to refer to one or to more than one (i.e., to at least one) of the grammatical object of the article. By way of example, “an element” means one element or more than one element.

As used herein, the term “about” modifying the quantity of an ingredient, parameter, calculation, or measurement in the compositions employed in the methods of the disclosure refers to the variation in the numerical quantity that can occur, for example, through typical measuring and liquid handling procedures used for making isolated polypeptides or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be typically within 10%, more typically still within 5%, of a given value or range. The term “about” also encompasses amounts that differ due to different equilibrium conditions for a composition resulting from a particular initial mixture. Whether or not modified by the term “about,” the paragraphs include equivalents to the quantities. Reference to “about” a value or parameter herein includes (and describes) embodiments that are directed to that value or parameter per se. For example, description referring to “about X” includes description of “X.” Numeric ranges are inclusive of the numbers defining the range.

The term “or” as used herein should be understood to mean “and/or,” unless the context clearly indicates otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The disclosure of a range should also be considered as disclosure of the endpoints of that range.

Exemplary methods and materials are described herein, although methods and materials similar or equivalent to those described herein can also be used in the practice or testing of the present application. The materials, methods, and examples are illustrative only and not intended to be limiting.

### Definitions

The following terms, unless otherwise indicated, shall be understood to have the following meanings:

The term “ablation,” as used herein, refers to a decrease or removal of activity. Thus, for example, “ablating FcγR binding” means that the Fc region amino acid variant has less than 50% starting binding as compared to an Fc region not containing the specific variant, with less than 70%, less than 80%, less than 90%, less than 95% or less than 98% loss of activity being preferred, and in general, with the activity being below the level of detectable binding in a BIACORE® assay (Pharmacia Biosensor AB, Uppsala, Sweden and Piscataway, N.J.). Unless otherwise noted, the Fe domains described herein retain binding to the FcRn receptor.

“Administering” or “administration of” a substance, a compound or an agent to a subject refers to the contact of that substance, compound or agent to the subject or a cell, tissue, organ or bodily fluid of the subject. Such administration can be carried out using one of a variety of methods known to those skilled in the art. For example, a compound or an agent can be administered intravenously or subcutaneously. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a subject to self-administer a drug, or to have the drug administered by another and/or who provides a subject with a prescription for a drug is administering the drug to the subject.

As used herein, the term “affinity” of a molecule refers to the strength of interaction between the molecule and a binding partner, such as a receptor, a ligand or an antigen. A molecule's affinity for its binding partner is typically expressed as the binding affinity equilibrium dissociation constant (K_{D}) of a particular interaction, wherein the lower the K_{D}, the higher the affinity. A K_{D} binding affinity constant can be measured by surface plasmon resonance, for example using the BIACORE® system (Pharmacia Biosensor AB, Uppsala, Sweden and Piscataway, N.J.) See also, Jonsson et al., Ann. Biol. Clin. 51:19 26 (1993); Jonsson et al., Biotechniques 11:620 627 (1991); Jonsson et al., J. Mol. Recognit. 8:125 131 (1995); Johnsson et al., Anal. Biochem. 198:268 277 (1991); Hearty S et al., Methods Mol Biol. 907:411-42 (2012), each incorporated herein by reference. The K_{D} may also be measured using a KinExA® system (Sapidyne Instruments, Hanover, Germany and Boise, ID). In some embodiments, the IL-15 variant of the heterodimeric protein described herein has reduced binding affinity towards IL-2/IL-15βγ receptor, compared with wild-type IL-15. In some embodiments, the first and/or the second Fc variant of the heterodimeric protein described herein has reduced affinity towards human, cynomolgus monkey, and mouse Fcγ receptors. In some embodiments, the first and/or the second Fc variant of the heterodimeric protein described herein does not bind to human, cynomolgus monkey, and mouse Fcγ receptors.

The terms “amino acid” and “amino acid identity,” as used herein, refer to one of the 20 naturally occurring amino acids that are coded for by DNA and RNA.

The term “amino acid substitution” or “substitution,” as used herein, refers to the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not considered an amino acid substitution.

The terms “amino acid insertion,” “amino acid addition” or “addition” or “insertion,” as used herein, refer to the addition of an amino acid sequence at a particular position in a parent polypeptide sequence. For example, −233E, _233E or 233E designates an insertion of glutamic acid after position 233 and before position 234. Additionally, −233ADE, _233ADE or 233ADE designates an insertion of AlaAspGlu after position 233 and before position 234.

The term “amino acid deletion” or “deletion,” as used herein, refers to the removal of an amino acid sequence at a particular position in a parent polypeptide sequence. For example, E233- or E233 #, E233( ), E233 or E233del designates a deletion of glutamic acid at position 233. Additionally, EDA233-, EDA233 or EDA233 #designates a deletion of the sequence GluAspAla that begins at position 233.

As used herein, the term “antibody” or “Ab” refers to an immunoglobulin molecule (e.g., complete antibodies, antibody fragment or modified antibodies) capable of recognizing and binding to a specific target or antigen, such as a carbohydrate, polynucleotide, lipid, polypeptide, etc., through at least one antigen recognition site, located in the variable region of the immunoglobulin molecule. As used herein, the term “antibody” can encompass any type of antibody, including but not limited to monoclonal antibodies, polyclonal antibodies, human antibodies, engineered antibodies (including humanized antibodies, fully human antibodies, chimeric antibodies, single-chain antibodies, artificially selected antibodies, CDR-granted antibodies, etc.) that specifically bind to a given antigen. In some embodiments, “antibody” and/or “immunoglobulin” (Ig) refers to a polypeptide comprising at least two heavy (H) chains (about 50-70 kDa) and two light (L) chains (about 25 kDa), optionally inter-connected by disulfide bonds. There are two types of light chain: λ and κ. In humans, λ and κ light chains are similar, but only one type is present in each antibody. Heavy chains are classified as mu, delta, gamma, alpha, or epsilon, and define the antibody's isotype as IgM, IgD, IgG, IgA, and IgE, respectively. See generally, Fundamental Immunology Ch. 7 (Paul, W., ed., 2nd ed. Raven Press, N.Y. (1989)) (incorporated by reference in its entirety). The methods, uses, and compositions-for-use disclosed herein utilize IgG antibodies.

As used herein, the term “checkpoint inhibitor” refers to a compound which targets and blocks checkpoint proteins. A checkpoint inhibitor interferes with the interaction between a checkpoint protein and its partner protein. Examples of checkpoint inhibitors include, but are not limited, to agents that target the PD-1/PD-L1 axis and agents that target CTLA-4.

As used herein, the term “cycle” refers to each administration event in a series of regularly repeated administration steps. For example, if a therapeutic agent (e.g. a heterodimeric protein of the present disclosure) is administered once every two weeks (Q2W), the first cycle begins on day 1 and ends on day 14, the second cycle begins on day 15 and ends on day 28, the third cycle begins on day 29 and ends on day 42, and so on. Measurements may be taken, and combination therapies may be administered, mid-cycle. Mid-cycle events can be defined by the cycle and the day in the cycle in which they occur, e.g., a measurement taken one week into a two-week cycle might be numbered Cycle 1, Day 8 (or C1D8). For combination therapies, a cycle will be defined by the period in which it takes the administration pattern to repeat. For example, if a first therapeutic agent is administered Q2W and a second therapeutic agent is administered Q1W, then the cycle is a two-week cycle. In such a case, if both agents are administered on C1D1, then the second dose of the second agent would be administered on C1D8, and the second dose of the first agent combined with the third dose of the second agent would be administered one week later to start the second cycle (i.e. C2D1). If a first therapeutic agent is administered Q1W and a second therapeutic agent is administered every three days (Q3D), then it will take three weeks for the administration pattern to repeat, so the cycle would be a three-week cycle and would include 3 administrations of the first agent and seven administrations of the second agent.

As used herein, the term “effector function” refers to a biochemical event that results from the interaction of an antibody Fc region with an Fc receptor or another effector molecule (e.g., Fc receptor-Like (FcRL) molecules, complement component C1q, and Tripartite motif-containing protein 21 (TRIM21)). Effector functions include, but are not limited to, antibody dependent cell-mediated cytotoxicity (ADCC), antibody dependent cell-mediated phagocytosis (ADCP) and complement-dependent cellular cytotoxicity (CDC). The term “ADCC” or “antibody dependent cell-mediated cytotoxicity,” as used herein, refers to the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity. As is discussed herein, many embodiments of the present disclosure ablate ADCC activity entirely. The term “ADCP” or “antibody dependent cell-mediated phagocytosis,” as used herein, refers to the cell-mediated reaction wherein nonspecific cytotoxic cells that express FcγRs recognize bound antibody on a target cell and subsequently cause phagocytosis of the target cell. The term “CDC” or “complement-dependent cellular cytotoxicity,” as used herein, refers to an effector function which leads to the activation of the classical complement pathway, which is triggered by the binding of an antibody to an antigen on the target cell, which activates a series of cascades containing complement-related protein groups in blood.

As used herein, the terms “Fc,” “Fc region” or “Fc domain” are used interchangeably herein and refer to the polypeptide comprising the constant region of an antibody excluding, in some instances, the first constant region immunoglobulin domain (e.g., CH1) or a portion thereof, and in some cases, part of the hinge. For IgG, the Fc domain comprises immunoglobulin domains Cγ2 and Cγ3 (Cγ2 and Cγ3) and the lower hinge region between Cγ1 (Cγ1) and Cγ2 (Cγ2). In some embodiments, an Fc refers to a truncated CH1 domain, and CH2 and CH3 of an immunoglobulin. Although the boundaries of the Fc region may vary, the human IgG heavy chain Fc region is usually defined to include residues E216 or C226 or P230 to its carboxyl-terminus, wherein the numbering is according to the EU numbering. In some embodiments, as is more fully described herein, amino acid modifications are made to the Fc region, for example to alter binding to one or more FcγR receptors or to the FcRn receptor. In some embodiments, the Fc domain is derived from a human IgG1 heavy chain Fc domain. In some embodiments, the Fc domain is derived from a human IgG2 heavy chain Fc domain. The “EU format as set forth in Edelman” or “EU numbering” or “EU index” refers to the residue numbering of the human Fc domain as described in Edelman G M et al. (Proc. Natl. Acad. USA (1969), 63, 78-85, hereby entirely incorporated by reference).

As used herein, the terms “Fc fusion protein” and “immunoadhesin” are used interchangeably and refer to a protein comprising an Fc region, generally linked (optionally through a linker moiety, as described herein) to a different protein, such as to IL-15 and/or IL-15R, as described herein. In some instances, two Fc fusion proteins can form a homodimeric Fc fusion protein or a heterodimeric Fc fusion protein with the latter being preferred.

As used herein, the term “Fe variant” or “variant Fc” refers to a protein comprising an amino acid modification in an Fc domain. The Fc variants of the present invention are defined according to the amino acid modifications that compose them. Thus, for example, N434S is an Fc variant with the substitution serine at position 434 relative to the parent Fc polypeptide, wherein the numbering is according to the EU index. Likewise, M428L/N434S defines an Fe variant with the substitutions M428L and N434S relative to the parent Fe polypeptide. For all positions discussed in the present invention that relate to antibodies, unless otherwise noted, amino acid position numbering is according to the EU index. The modification can be an addition, deletion, or substitution. Substitutions can include naturally occurring amino acids and, in some cases, synthetic amino acids. Examples include, but are not limited to, U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, all of them entirely incorporated by reference. In some embodiments, the substitutions comprise only naturally occurring amino acids. In some embodiments, the substitutions do not comprise any synthetic amino acids.

The terms “Fc gamma receptor,” “FcγR” and “FcgammaR,” as used herein, are used interchangeably and refer to any member of the family of proteins that bind the IgG antibody Fc region and is encoded by an FcγR gene. An FcγR may be from any organism. In some embodiments, the FcγR is a human FcγR. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes.

The term “FcRn” or “neonatal Fc Receptor,” as used herein, refers to a protein that binds the IgG antibody Fc region and is encoded at least in part by an FcRn gene. The FcRn may be from any organism. In some embodiments, the FcRn is a human FcRn. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants can be used to increase binding to the FcRn receptor, and in some cases, to increase serum half-life. In general, unless otherwise noted, the Fc monomers disclosed herein retain binding to the FcRn receptor (and, as noted below, can include amino acid variants to increase binding to the FcRn receptor).

The term “modification,” as used herein, refers to an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always referring to an amino acid coded for by DNA, e.g., the 20 amino acids that have codons in DNA and RNA.

The terms “nucleic acid,” “polynucleotide” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T. In some embodiments, the polynucleotide comprises only natural nucleotides. In some embodiments, the polynucleotide does not comprise any analogues of a natural nucleotide.

The term “non-naturally occurring modification,” as used herein, refers to an amino acid modification that is not isotypic. For example, because none of the IgGs comprise a serine at position 434, the substitution 434S in IgG1, IgG2, IgG3, or IgG4 (or hybrids thereof) is considered a non-naturally occurring modification.

The terms “patient,” “subject” and “individual” are used interchangeably herein and refer to either a human or a non-human animal in need to treatment. These terms include mammals, such as humans, and primates (e.g., monkey). In some embodiments, the subject is a human. In some embodiments, the subject is in need of treatment of a blood cancer, such as multiple myeloma. The terms “treating” and “treatment,” as used herein, refer to reduction in severity and/or frequency of symptoms, elimination of symptoms and/or underlying cause, prevention of the occurrence of symptoms and/or their underlying cause, and improvement or remediation of damage.

As used herein, “percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Pub. No. 20160244525, hereby incorporated by reference.

As used herein, the terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids. In some embodiments, the polypeptide only comprises naturally-occurring amino acids. In some embodiments, the polypeptide does not comprise any chemical analogues or modified derivatives of a corresponding naturally-occurring amino acids. Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are known in the art.

The term “position,” as used herein, refers to a location in the sequence of a protein. Positions may be numbered sequentially, or according to an established format, for example the EU index for antibody numbering. A position may be defined relative to a reference sequence. In such cases, the reference sequence is provided for comparison purposes, and the heterodimeric protein of the disclosure (or a portion thereof) may comprise additional amino acid alterations (e.g., substitutions, insertions, and deletions) relative to the reference sequence. In some embodiments, the heterodimeric protein of the disclosure (or a portion thereof) does not comprise any additional amino acid alterations relative to the reference sequence.

The term “residue,” as used herein, refers to a position in a protein and its associated amino acid identity. For example, Asparagine 297 (also referred to as Asn297 or N297) is a residue at position 297 in a specific protein.

As used herein, the term “therapeutically effective amount” and “effective amount” are used interchangeably herein and refer to that amount of the therapeutic agent being administered, as a single agent or in combination with one or more additional agents, which will relieve to some extent one or more of the symptoms of the condition being treated. In some embodiments, the therapeutically effective amount is an amount sufficient to effect the beneficial or desired clinical results. With respect to the treatment of cancer, a therapeutically effective amount refers to that amount which has at least one of the following effects: palliate, ameliorate, stabilize, reverse, prevent, slow or delay the progression of (and/or symptoms associated with) of a blood cancer, such as multiple myeloma. The effective amounts that may be used in the present disclosure varies depending upon the manner of administration, the age, body weight, and general health of the subject. The appropriate amount and dosage regimen can be determined using routine skill in the art. For example, efficacy can be determined using the International Myeloma Working Group (IMWG) Uniform Response Criteria.

The terms “wild type” or “WT” are used interchangeably herein and refer to an amino acid sequence or a nucleotide sequence that is found in nature, including allelic variations. A WT protein has an amino acid sequence or is encoded by a nucleotide sequence that has not been intentionally modified.

### General

The present disclosure relates to methods of treating a blood cancer in a subject in need thereof, the method comprising administering to the subject a therapeutically effective amount of a heterodimeric Fc fusion protein (or a combination of heterodimeric Fc fusion proteins) that includes IL-15 and IL-15 receptor alpha (IL-15Rα) protein domains. The present disclosure relates to methods for inducing the proliferation of CD8⁺ effector memory T cells and/or NK cells in a subject suffering from a blood cancer or for inducing IFNγ production in a subject suffering from a blood cancer, the method comprising administering to the subject an effective amount of a heterodimeric Fc fusion protein (or a combination of heterodimeric Fc fusion proteins) that includes IL-15 and IL-15 receptor alpha (IL-15Rα) protein domains. The Fc domains can be derived from IgG Fc domains, e.g., IgG1, IgG2, IgG3 or IgG4 Fe domains.

### IL15-IL15Rα Heterodimeric Fc-Fusion Proteins

Any of the IL15-IL15Rα heterodimeric Fe-fusion proteins disclosed in US2018/0118805, the entire disclosure of which is incorporated by reference herein, or a combination thereof, may be used in the methods disclosed herein. These include, inter alia, the Fe variants such as steric variants (e.g., “knob-into-holes,” “skew,” “electrostatic steering,” “charged pairs” variants), pI variants, isotypic variants, FcγR variants, and ablation variants (e.g., “FcγR ablation variants” or “Fc knock out (FcKO or KO)” variants) as well as the various IL-15 and IL15Rα proteins disclosed therein.

Thus, in some embodiments, the heterodimeric protein useful in the methods disclosed herein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains, respectively, comprise a set of amino acid substitutions selected from the group consisting of S267K/L368D/K370S:S267K/S364K/E357Q; S364K/E357Q:L368D/K370S; L368D/K370S:S364K; L368E/K370S:S364K; T411E/K360E/Q362E:D401K; L368D/K370S:S364K/E357L; K370S:S364K/E357Q; S267K/S364K/E357Q:S267K/L368D/K370S; L368D/K370S:S364K/E357Q; S364K:L368D/K370S; S364K:L368E/K370S; D401K:T411E/K360E/Q362E; S364K/E357L:L368D/K370S; and S364K/E357Q:K370S, according to EU numbering.

In some embodiments, said first and said second Fc domains, respectively, comprise the S267K/L368D/K370S:S267K/S364K/E357Q set of amino acid substitutions, according to EU numbering. In some embodiments, said first and said second Fc domains, respectively, comprise the S364K/E357Q:L368D/K370S set of amino acid substitutions, according to EU numbering. In some embodiments, said first and said second Fc domains, respectively, comprise the L368D/K370S:S364K set of amino acid substitutions, according to EU numbering. In some embodiments, said first and said second Fc domains, respectively, comprise the L368E/K370S:S364K set of amino acid substitutions, according to EU numbering. In some embodiments, said first and said second Fc domains, respectively, comprise the T411E/K360E/Q362E:D401K set of amino acid substitutions, according to EU numbering. In some embodiments, said first and said second Fc domains, respectively, comprise the L368D/K370S:S364K/E357L set of amino acid substitutions, according to EU numbering. In some embodiments, said first and said second Fc domains, respectively, comprise the K370S:S364K/E357Q set of amino acid substitutions, according to EU numbering. In some embodiments, said first and said second Fe domains, respectively, comprise the S267K/S364K/E357Q:S267K/L368D/K370S set of amino acid substitutions, according to EU numbering. In some embodiments, said first and said second Fc domains, respectively, comprise the L368D/K370S:S364K/E357Q set of amino acid substitutions, according to EU numbering. In some embodiments, said first and said second Fc domains, respectively, comprise the S364K:L368D/K370S set of amino acid substitutions, according to EU numbering. In some embodiments, said first and said second Fc domains, respectively, comprise the S364K:L368E/K370S set of amino acid substitutions, according to EU numbering. In some embodiments, said first and said second Fc domains, respectively, comprise the D401K:T411E/K360E/Q362E set of amino acid substitutions, according to EU numbering. In some embodiments, said first and said second Fc domains, respectively, comprise the S364K/E357L:L368D/K370S set of amino acid substitutions, according to EU numbering. In some embodiments, said first and said second Fc domains, respectively, comprise the S364K/E357Q:K370S set of amino acid substitutions, according to EU numbering.

In some embodiments, each of said first and/or second Fc domains, independently, further comprises amino acid substitutions selected from the group consisting of Q295E, N384D, Q418E and N421D, or a combination thereof according to EU numbering. In some embodiments, the first Fc domain further comprises amino acid substitutions selected from the group consisting of Q295E, N384D, Q418E and N421D, or a combination thereof, according to EU numbering. In some embodiments, the second Fc domain further comprises amino acid substitutions selected from the group consisting of Q295E, N384D, Q418E and N421D, or a combination thereof, according to EU numbering. In some embodiments, each of said first and second Fe domains further comprises amino acid substitutions selected from the group consisting of Q295E, N384D, Q418E and N421D, or a combination thereof, according to EU numbering. In some embodiments, the first Fc domain further comprises amino acid substitutions Q295E, N384D, Q418E and N421D, according to EU numbering. In some embodiments, the second Fc domain further comprises amino acid substitutions Q295E, N384D, Q418E and N421D, according to EU numbering. In some embodiments, each of said first and second Fc domains further comprises amino acid substitutions Q295E, N384D, Q418E and N421D, according to EU numbering.

In some embodiments, the first Fc domain does not comprise a free Cysteine at position 220, according to EU numbering. In some embodiments, the first Fc domain comprises the amino acid substitution C220S, according to EU numbering. In some embodiments, the second Fc domain does not comprise a free Cysteine at position 220, according to EU numbering. In some embodiments, the second Fc domain comprises the amino acid substitution C220S, according to EU numbering. In some embodiments, the first and second Fc domains do not comprise a free Cysteine at position 220, according to EU numbering. In some embodiments, the first and second Fc domains both comprise the amino acid substitution C220S, according to EU numbering.

In some embodiments, the first Fc domain further comprises one or more amino acid substitutions selected from the group consisting of E233P, L234V, L235A, G236del, G236R, S239K, S267K, A327G, and L328R or a combination thereof, according to EU numbering. In some embodiments, the first Fc domain further comprises amino acid substitutions E233P, L234V, L235A, G236del, and S267K, according to EU numbering. In some embodiments, the second Fc domain further comprises one or more amino acid substitutions selected from the group consisting of E233P, L234V, L235A, G236del, G236R, S239K, S267K, A327G, and L328R, or a combination thereof, according to EU numbering. In some embodiments, the second Fc domain further comprises amino acid substitutions E233P, L234V, L235A, G236del, and S267K, according to EU numbering. In some embodiments, the first and second Fc domains each comprise amino acid substitutions E233P, L234V, L235A, G236del, and S267K, according to EU numbering.

The position of the various Fc domain substitutions is in reference to the corresponding position in the wild-type IgG1 Fc domain (SEQ ID NO: 12). The amino acid sequence of the wild-type IgG1 Fc domain (SEQ ID NO: 12) is an exemplary sequence provided for comparison purposes, and the Fc domain of the heterodimeric protein may comprise additional amino acid alterations (e.g., substitutions, insertions, and deletions) relative to the wild-type IgG1 Fc domain (SEQ ID NO: 12). For example, the Fc domain of the heterodimeric protein may be derived from a different wild-type human IgG1 allele. In some embodiments, the Fc domain of the heterodimeric protein does not comprise any additional amino acid alterations relative to the wild-type IgG1 Fc domain (SEQ ID NO: 12). The skilled artisan would be able to determine the corresponding substitutions in an Fc domain derived from an IgG2, an IgG3 or an IgG4 Fc domain. For example, the skilled artisan would recognize that residues E233, L234, L235 and G236 are present in Fc domains derived from IgG1 or IgG3 Fc domains. In some embodiments, the position of the various Fc domain substitutions is in reference to the corresponding position in the wild-type IgG3 Fc domain (SEQ ID NO: 14). The amino acid sequence of the wild-type IgG3 Fc domain (SEQ ID NO: 14) is an exemplary sequence provided for comparison purposes, and the Fc domain of the heterodimeric protein may comprise additional amino acid alterations (e.g., substitutions, insertions, and deletions) relative to the wild-type IgG3 Fc domain (SEQ ID NO: 14). For example, the Fc domain of the heterodimeric protein may be derived from a different wild-type human IgG3 allele. In some embodiments, the Fc domain of the heterodimeric protein does not comprise any additional amino acid alterations relative to the wild-type IgG3 Fc domain (SEQ ID NO: 14).

In some embodiments, each of said first and/or second Fc domains, independently, further comprises amino acid substitutions selected from the group consisting of G236R/L328R; E233P/L234V/L235A/G236del/S239K; E233P/L234V/L235A/G236del/S267K; E233P/L234V/L235A/G236del/S239K/A327G; E233P/L234V/L235A/G236del/S267K/A327G; and E233P/L234V/L235A/G236del, according to EU numbering and wherein the Fc domains are derived from IgG1 or IgG3 Fc domains. In some embodiments, said first Fc domain further comprises amino acid substitutions selected from the group consisting of G236R/L328R; E233P/L234V/L235A/G236del/S239K; E233P/L234V/L235A/G236del/S267K; E233P/L234V/L235A/G236del/S239K/A327G; E233P/L234V/L235A/G236del/S267K/A327G; and E233P/L234V/L235A/G236del, according to EU numbering and wherein the Fc domains are derived from IgG1 or IgG3 Fc domains. In some embodiments, said second Fc domain further comprises amino acid substitutions selected from the group consisting of G236R/L328R; E233P/L234V/L235A/G236del/S239K; E233P/L234V/L235A/G236del/S267K; E233P/L234V/L235A/G236del/S239K/A327G; E233P/L234V/L235A/G236del/S267K/A327G; and E233P/L234V/L235A/G236del, according to EU numbering and wherein the Fc domains are derived from IgG1 or IgG3 Fc domains. In some embodiments, said first and second Fc domains further comprise amino acid substitutions selected from the group consisting of G236R/L328R; E233P/L234V/L235A/G236del/S239K; E233P/L234V/L235A/G236del/S267K; E233P/L234V/L235A/G236del/S239K/A327G; E233P/L234V/L235A/G236del/S267K/A327G; and E233P/L234V/L235A/G236del, according to EU numbering and wherein the Fc domains are derived from IgG1 or IgG3 Fc domains.

The skilled artisan would also recognize that the corresponding residues in a Fc domain derived IgG2 Fc domain are P233, V234, and A235 and that an Fc domain derived from IgG2 lacks a residue corresponding to residue G236. Accordingly, the skilled artisan would recognize that reference to E233P, L234V, L235A, and G236del herein is a reference to P233, V234, A235 and −236 if the Fc domain is derived from an IgG2 Fc domain (i.e., the PVA-sequence present in wild type IgG2). In some embodiments, the position of the various Fc domain substitutions is in reference to the corresponding position in the wild-type IgG2 Fc domain (SEQ ID NO: 13). The amino acid sequence of the wild-type IgG2 Fc domain (SEQ ID NO: 13) is an exemplary sequence provided for comparison purposes, and the Fc portion of the heterodimeric protein may comprise additional amino acid alterations (e.g., substitutions, insertions, and deletions) relative to the wild-type IgG2 Fc domain (SEQ ID NO: 13). For example, the Fc domain of the heterodimeric protein may be derived from a different wild-type human IgG2 allele. In some embodiments, the Fc domain of the heterodimeric protein does not comprise any additional amino acid alterations relative to the wild-type IgG2 Fc domain (SEQ ID NO: 13).

In some embodiments, each of said first and/or second Fc domains, independently, further comprises amino acid substitutions selected from the group consisting of L328R; S239K; S267K; S239K/A327G; and S267K/A327G, according to EU numbering and wherein the Fc domains are derived from an IgG2 Fc domain. In some embodiments, said first Fc domain further comprises amino acid substitutions selected from the group consisting of L328R; S239K; S267K; S239K/A327G; and S267K/A327G, according to EU numbering and wherein the Fc domains are derived from an IgG2 Fc domain. In some embodiments, said second Fc domain further comprises amino acid substitutions selected from the group consisting of L328R; S239K; S267K; S239K/A327G; and S267K/A327G, according to EU numbering and wherein the Fc domains are derived from an IgG2 Fc domain. In some embodiments, said first and second Fc domains further comprise amino acid substitutions selected from the group consisting of L328R; S239K; S267K; S239K/A327G; and S267K/A327G, according to EU numbering and wherein the Fc domains are derived from an IgG2 Fc domain.

The skilled artisan would also recognize that in a Fc domain derived from an IgG4, residue 234 is a phenylalanine. Accordingly, the skilled artisan would recognize that reference to L234 herein (e.g., L234V) is a reference to F234 (e.g., F234V) if the Fc domain is derived from an IgG4 Fc domain. In some embodiments, the position of the various Fc domain substitutions is in reference to the corresponding position in the wild-type IgG4 Fc domain (SEQ ID NO: 15). The amino acid sequence of the wild-type IgG4 Fc domain (SEQ ID NO: 15) is an exemplary sequence provided for comparison purposes, and the Fc domain of the heterodimeric protein may comprise additional amino acid alterations (e.g., substitutions, insertions, and deletions) relative to the wild-type IgG4 Fc domain (SEQ ID NO: 15). For example, the Fc domain of the heterodimeric protein may be derived from a different wild-type human IgG4 allele. In some embodiments, the Fc domain of the heterodimeric protein does not comprise any additional amino acid alterations relative to the wild-type IgG4 Fc domain (SEQ ID NO: 15).

In some embodiments, each of said first and/or second Fc domains, independently, further comprises amino acid substitutions selected from the group consisting of G236R/L328R; E233P/F234V/L235A/G236del/S239K; E233P/F234V/L235A/G236del/S267K; E233P/F234V/L235A/G236del/S239K/A327G; E233P/F234V/L235A/G236del/S267K/A327G; and E233P/F234V/L235A/G236del, according to EU numbering and wherein the Fc domains are derived from IgG4 Fc domain. In some embodiments, said first Fc domain further comprises amino acid substitutions selected from the group consisting of G236R/L328R; E233P/F234V/L235A/G236del/S239K; E233P/F234V/L235A/G236del/S267K; E233P/F234V/L235A/G236del/S239K/A327G; E233P/F234V/L235A/G236del/S267K/A327G; and E233P/F234V/L235A/G236del, according to EU numbering and wherein the Fc domains are derived from IgG4 Fc domain. In some embodiments, said second Fc domain further comprises amino acid substitutions selected from the group consisting of G236R/L328R; E233P/F234V/L235A/G236del/S239K; E233P/F234V/L235A/G236del/S267K; E233P/F234V/L235A/G236del/S239K/A327G; E233P/F234V/L235A/G236del/S267K/A327G; and E233P/F234V/L235A/G236del, according to EU numbering and wherein the Fc domains are derived from IgG4 Fc domain. In some embodiments, said first and second Fc domains further comprise amino acid substitutions selected from the group consisting of G236R/L328R; E233P/F234V/L235A/G236del/S239K; E233P/F234V/L235A/G236del/S267K; E233P/F234V/L235A/G236del/S239K/A327G; E233P/F234V/L235A/G236del/S267K/A327G; and E233P/F234V/L235A/G236del, according to EU numbering and wherein the Fc domains are derived from IgG4 Fc domain.

In some embodiments, the first Fc domain further comprises the amino acid substitution M428L or N434S, according to EU numbering. In some embodiments, the first Fc domain further comprises the amino acid substitution M428L, according to EU numbering. In some embodiments, the first Fc domain further comprises the amino acid substitution N434S, according to EU numbering. In some embodiments, the second Fc domain further comprises the amino acid substitution M428L or N434S, according to EU numbering. In some embodiments, the second Fc domain further comprises the amino acid substitution M428L, according to EU numbering. In some embodiments, the second Fc domain further comprises the amino acid substitution N434S, according to EU numbering. In some embodiments, the first Fc domain further comprises amino acid substitutions M428L and N434S, according to EU numbering. In some embodiments, the second Fc domain further comprises amino acid substitutions M428L and N434S, according to EU numbering. In some embodiments, the first and second Fc domains each further comprise amino acid substitutions M428L and N434S, according to EU numbering. In some embodiments, the first and second Fc domains each further comprise the amino acid substitution M428L, according to EU numbering. In some embodiments, the first and second Fe domains each further comprise the amino acid substitution N434S, according to EU numbering.

In some embodiments, said first and/or second Fc domain further comprises amino acid substitution K246T, according to EU numbering. In some embodiments, the first Fc domain further comprises amino acid substitution K246T, according to EU numbering. In some embodiments, the second Fc domain further comprises amino acid substitution K246T, according to EU numbering. When the K246T substitution appears in the second Fc domain, it may also be called a K100T mutation based on the amino acid numbering of the second monomer (see, e.g., SEQ ID NO: 10 and 16). In some embodiments, the first and second Fc domains further comprise amino acid substitution K246T, according to EU numbering.

In some embodiments, the first Fc domain comprises amino acid substitutions L368D and K370S; the second Fc domain comprises amino acid substitutions S364K and E357Q; and each of said first and second Fc domains further comprises amino acid substitutions C220S, E233P, L234V, L235A, G236del, S267K, M428L and N434S, all according to EU numbering. In some embodiments, the first Fc domain comprises amino acid substitutions S364K and E357Q; the second Fc domain comprises amino acid substitutions L368D and K370S; and each of said first and second Fc domains further comprises amino acid substitutions C220S, E233P, L234V, L235A, G236del, S267K, M428L and N434S, all according to EU numbering. In some embodiments, the first Fc domain comprises amino acid substitutions L368D and K370S; the second Fc domain further comprises amino acid substitutions S364K and E357Q; and each of said first and second Fc domains further comprises amino acid substitutions C220S, E233P, L234V, L235A, G236del, S267K, M428L and N434S, all according to EU numbering; wherein said IL-15 protein comprises amino acid substitutions D30N, E64Q and N65D; and wherein said IL-15Rα protein comprises SEQ ID NO:4. In some embodiments, the first Fc domain comprises amino acid substitutions S364K and E357Q; the second Fc domain comprises amino acid substitutions L368D and K370S; and each of said first and second Fc domains further comprises amino acid substitutions C220S, E233P, L234V, L235A, G236del, S267K, M428L and N434S, all according to EU numbering; wherein said IL-15 protein comprises amino acid substitutions D30N, E64Q and N65D; and wherein said IL-15Rα protein comprises SEQ ID NO:4.

In some embodiments, the first Fc domain comprises amino acid substitutions L368D and K370S; the second Fc domain comprises amino acid substitutions K246T, S364K, and E357Q; and each of said first and second Fc domains further comprises amino acid substitutions C220S, E233P, L234V, L235A, G236del, S267K, M428L and N434S, all according to EU numbering. In some embodiments, the first Fc domain comprises amino acid substitutions S364K and E357Q; the second Fc domain comprises amino acid substitutions K246T, L368D and K370S; and each of said first and second Fc domains further comprises amino acid substitutions C220S, E233P, L234V, L235A, G236del, S267K, M428L and N434S, all according to EU numbering. In some embodiments, the first Fc domain comprises amino acid substitutions L368D and K370S; the second Fc domain comprises amino acid substitutions K246T, S364K and E357Q; and each of said first and second Fc domains further comprises amino acid substitutions C220S, E233P, L234V, L235A, G236del, S267K, M428L and N434S, all according to EU numbering; wherein said ILL-15 protein comprises amino acid substitutions D30N, E64Q and N65D; and wherein said IL-15Rα protein comprises SEQ ID NO:4. In some embodiments, the first Fc domain comprises amino acid substitutions S364K and E357Q; the second Fc domain comprises amino acid substitutions K246T, L368D and K370S; and each of said first and second Fc domains further comprises amino acid substitutions C220S, E233P, L234V, L235A, G236del, S267K, M428L and N434S, all according to EU numbering; wherein said IL-15 protein comprises amino acid substitutions D30N, E64Q and N65D; and wherein said IL-15Rα protein comprises SEQ ID NO:4.

In some embodiments, the first Fc domain of the heterodimeric protein comprises the sequence set forth in SEQ ID NO: 6. In some embodiments, the second Fc domain of the heterodimeric protein comprises the sequence set forth in SEQ ID NO: 7. In some embodiments, the second Fc domain of the heterodimeric protein comprises the sequence set forth in SEQ ID NO: 8.

In some embodiments, any one of the amino acid substitutions of the Fc variant domains described herein is on one of the monomers or on both monomers (e.g., on the first Fc domain; on the second Fc domain or on both Fc domains).

In some embodiments, the Fc domain of the first monomer is derived from IgG1, IgG2, IgG3, or IgG4. In some embodiments, the Fc domain of the first monomer is derived from IgG1. In some embodiments, the Fc domain of the first monomer is derived from IgG2. In some embodiments, the Fc domain of the first monomer is derived from IgG3. In some embodiments, the Fc domain of the first monomer is derived from IgG4. In some embodiments, the Fc domain of the second monomer is derived from IgG1, IgG2, IgG3, or IgG4. In some embodiments, the Fc domain of the second monomer is derived from IgG1. In some embodiments, the Fc domain of the second monomer is derived from IgG2. In some embodiments, the Fc domain of the second monomer is derived from IgG3. In some embodiments, the Fc domain of the second monomer is derived from IgG4.

As used herein, “IL-15,” “IL15” or “Interleukin 15” may be used interchangeably and refer to a four-α-helix protein belonging to a family of cytokines. IL-15 signals through a receptor complex composed of the IL-2/IL-15 receptor 3 (IL-15Rβ) (CD122) subunit. In some embodiments, the IL-15 protein comprises the polypeptide sequence set forth in SEQ ID NO:2 (full-length human IL-15). In some embodiments, the IL-15 protein comprises the polypeptide sequence set forth in SEQ ID NO:1 (truncated or mature human IL-15). In some embodiments, the IL-15 protein comprises a polypeptide sequence selected from the group consisting of SEQ ID NO:1 and SEQ ID NO:2.

In some embodiments, the IL-15 protein of the first monomer is an IL-15 protein variant having a different amino acid sequence than wild type IL-15 protein (SEQ ID NO: 1). In some embodiments, the IL-15 variant is engineered to have reduced binding affinity (compared with wild-type IL-15) towards IL-2/IL-15βγ receptor complex with the goal of improving tolerability and extending pharmacokinetics by reducing acute toxicity, and ultimately promote antitumor immunity through IL-15 mediated signaling on CD8⁺ T cells and NK cells. In certain embodiments, the sequence of the IL-15 protein variant of the first monomer has at least one (i.e., 1, 2, 3, 4, 5, 6, 7, 8, 9, 10, or more) amino acid substitutions compared to the wild-type IL-15 sequence protein (SEQ ID NO: 1). In some embodiments, the amino acid substitution may include one or more of an amino acid substitution or deletion in the domain of IL-15 that interacts with IL-15R and/or IL-2/IL-15βγ receptor complex. In some embodiments, the amino acid substitution may include one or more of an amino acid substitution or deletion in the domain of IL-15 protein which causes a decreased binding affinity, compared with the affinity of a wild-type IL-15, towards IL-2/IL-15βγ receptor complex. In some embodiments, the IL-15 protein comprises one or more amino acid substitutions selected from the group consisting of N1D, N4D, D8N, D30N, D61N, E64Q, N65D and Q108E. In some embodiments, said IL15 protein comprises one or more amino acid substitutions selected from the group consisting of E87C, V49C, L52C, E89C, Q48C, E53C, C42S and L45C. The amino acid substitutions for the IL-15 protein disclosed herein are relative to wild-type IL-15 (mature form; SEQ ID NO: 1). The amino acid sequence of wild-type IL-15 (mature form; SEQ ID NO: 1) is an exemplary sequence provided for comparison purposes, and the IL-15 protein of the heterodimeric protein may comprise additional amino acid alterations (e.g., substitutions, insertions, and deletions) relative to wild-type IL-15. For example, the IL-15 protein of the heterodimeric protein may be derived from a different wild-type human IL-15 allele. In some embodiments, the IL-15 protein of the heterodimeric protein does not comprise any additional amino acid alterations relative to wild-type IL-15. In some embodiments, the IL-15 protein variant present in the first monomer comprises the amino acid sequence set forth in SEQ ID NO:5 (XENP24306/XENP32803). In some embodiments, the IL-15 protein comprises the amino acid sequence set forth in SEQ ID NO: 5.

In some embodiments, the IL-15 protein comprises amino acid substitutions D30N, E64Q and N65D. In some embodiments, the IL-15 protein comprises the following amino acid substitutions: N4D and N65D. In some embodiments, the IL-15 protein comprises the following amino acid substitutions: D30N and N65D. In some embodiments, the IL-15 protein present in the first monomer comprises an N65D amino acid substitution and one or more amino acid substitutions selected from the group consisting of N4D, D30N, E64Q. In some embodiments, the IL-15 protein present in the first monomer comprises an N65D amino acid substitution and one or more amino acid substitutions selected from the group consisting of N4D, D30N, E64Q. In some embodiments, the IL-15 protein present in the first monomer comprises an N65D amino acid substitution and consists of the amino acid substitutions N4D, D30N, E64Q. The amino acid substitutions for the IL-15 protein disclosed herein are relative to wild-type IL-15 (SEQ ID NO: 1). The amino acid sequence of wild-type IL-15 (SEQ ID NO: 1) is an exemplary sequence provided for comparison purposes, and the IL-15 protein of the heterodimeric protein may comprise additional amino acid alterations (e.g., substitutions, insertions, and deletions) relative to wild-type IL-15. For example, the IL-15 protein of the heterodimeric protein may be derived from a different wild-type human IL-15 allele. In some embodiments, the IL-15 protein of the heterodimeric protein does not comprise any additional amino acid alterations relative to wild-type IL-15.

IL-15Rα protein is a transmembrane protein with very high affinity for IL-15 that facilitates IL-15 trafficking from the endoplasmic reticulum (ER) through the cytoplasm and presentation of IL-15/IL-15Rα complexes on the cell surface. As used herein, the term “sushi domain of IL-15Rα” refers to the truncated extracellular region of IL-15Rα or recombinant human IL-15 receptor α. In some embodiments, the IL-15Rα protein comprises a polypeptide sequence of SEQ ID NO:3 (full-length human IL-15Rα). In some embodiments, the IL-15Rα protein comprises a polypeptide sequence of SEQ ID NO:4 (sushi domain of human IL-15Rα).

In some embodiments, said IL15Rα protein comprises one or more amino acid alterations selected from the group consisting of DPC or DCA insertions after residue 65 (65DPC or D96/P97/C98, 65DCA or D96/C97/A98), S40C, K34C, G38C, L42C and A37C. The numbering of these amino acid substitutions for the IL-15Rα protein is relative to the sushi domain of human IL-15Rα (SEQ ID NO: 4). The amino acid sequence of the sushi domain of human IL-15Rα (SEQ ID NO: 4) is an exemplary sequence provided for comparison purposes, and the IL-15Rα protein of the heterodimeric protein may comprise additional amino acid alterations (e.g., substitutions, insertions, and deletions) relative to the sushi domain of human IL-15Rα (SEQ ID NO: 4). For example, the IL-15Rα protein of the heterodimeric protein may be derived from a different wild-type human IL-15Rα allele. In some embodiments, the IL-15Rα protein of the heterodimeric protein does not comprise any additional amino acid alterations relative to the sushi domain of human IL-15Rα (SEQ ID NO: 4).

In some embodiments, 1115 protein and the IL15Rα protein comprise a set of amino acid substitutions or additions selected from the group consisting of E87C: 65DPC (DPC insertions after residue 65 or D96/P97/C98); E87C: 65DCA (DCA insertions after residue 65 or D96/C97/A98); V49C:S40C; L52C:S40C; E89C:K34C; Q48C:G38C; E53C:L42C; C42S:A37C; and L45C:A37C, respectively. The numbering of these amino acid substitutions for the IL-15Rα protein is relative to the sushi domain of human IL-15Rα (SEQ ID NO: 4). The amino acid sequence of the sushi domain of human TL-15Rα (SEQ ID NO: 4) is an exemplary sequence provided for comparison purposes, and the IL-15Rα protein of the heterodimeric protein may comprise additional amino acid alterations (e.g., substitutions, insertions, and deletions) relative to the sushi domain of human IL-15Rα (SEQ ID NO: 4). For example, the IL-15Rα of the heterodimeric protein may be derived from a different wild-type human IL-15Rα allele. In some embodiments, the IL-15Rα protein of the heterodimeric protein does not comprise any additional amino acid alterations relative to the sushi domain of human IL-15Rα (SEQ ID NO: 4).

In some embodiments, the IL-15Rα protein comprises a polypeptide sequence selected from the group consisting of SEQ ID NO:3 and SEQ ID NO: 4. In some embodiments, the IL-15Rα protein comprises the amino acid sequence of SEQ ID NO:3 (full-length human IL-15Rα). In some embodiments, the IL-15Rα protein comprises the amino acid sequence SEQ ID NO:4 (sushi domain of human IL-15Rα). In some embodiments, the IL-15 protein comprises amino acid substitutions D30N, E64Q and N65D; and the IL-15Rα protein comprises SEQ ID NO:4 (sushi domain of human IL-15Rα).

The heterodimeric protein of the disclosure is an IL-15/IL-15Rα-Fc heterodimeric fusion protein. The N-terminus of one side of the heterodimeric Fc domain is covalently attached to the C-terminus of IL-15 protein, while the other side is covalently attached to the sushi domain (truncated extracellular region) of IL-15Rα. In some embodiments, the IL-15 protein and IL-15Rα (sushi domain) may have a variable length linker between the C-terminus of IL-15 and IL-15Rac and the N-terminus of each of the Fc regions. In some embodiments, the IL-15 protein is covalently attached to the N-terminus of the first Fc domain via a first linker. In some embodiments, the IL-15Rα protein is covalently attached to the N-terminus of the second Fc domain using a second linker. In some embodiments, the IL-15 protein is covalently attached to the N-terminus of the first Fc domain via a first linker and the IL-15Rα protein is covalently attached to the N-terminus of the second Fc domain via a second linker. The term “linker,” as used herein, refers to a polypeptide sequence that joins two or more domains. The characteristics of linkers and their suitability for particular purposes are known in the art. See, e.g., Chen et al. Adv Drug Deliv Rev. October 15; 65(10): 1357-1369 (2013) (disclosing various types of linkers, their properties, and associated linker designing tools and databases), which is incorporated herein by reference. In some embodiments, the linker is flexible, rigid, or in vivo cleavable. In some embodiments, the linker is flexible. In some embodiments, the first linker and/or second linker is, independently, a variable length Gly-Ser linker. Flexible linkers typically comprise small non-polar amino acids (e.g. Gly) or polar amino acids (e.g., Ser or Thr). Examples of flexible linkers that can be used in the present disclosure are sequences consisting primarily of stretches of Gly and Ser residues (“GS” linker). In some embodiments, flexible linkers comprise repeats of 4 Gly and Ser residues. In some embodiments, the flexible linker comprises 1-5 repeats of five Gly and Ser residues. Non-limiting examples of flexible linker include (Gly-Gly-Gly-Gly-Ser)n (SEQ ID NO: 39), (Ser-Ser-Ser-Ser-Gly)n (SEQ ID NO: 40), (Gly-Ser-Ser-Gly-Gly)n (SEQ ID NO: 41), and (Gly-Gly-Ser-Gly-Gly)n (SEQ ID NO: 42), where n may be any integer between 1 and 5. In some embodiments, the linker is between 5 and 25 amino acid residues long. In some embodiments, the flexible linker comprises 5, 10, 15, 20, or 25 residues. Other suitable linkers may be selected from the group consisting of AS (SEQ ID NO: 43), AST (SEQ ID NO: 44), TVAAPS (SEQ ID NO: 45), TVA (SEQ ID NO: 46), ASTSGPS (SEQ ID NO: 47), KESGSVSSEQLAQFRSLD (SEQ ID NO: 48), EGKSSGSGSESKST (SEQ ID NO: 49), (Gly)6 (SEQ ID NO: 50), (Gly)8 (SEQ ID NO: 51), and GSAGSAAGSGEF (SEQ ID NO: 52). In general, a flexible linker provides good flexibility and solubility and may serve as a passive linker to keep a distance between functional domains. The length of the flexible linkers can be adjusted to allow for proper folding or to achieve optimal biological activity of the fusion proteins. In some embodiments, the linker comprises the sequence (Gly-Gly-Gly-Gly-Ser; SEQ ID NO: 53). In some embodiments, the first and second linker comprise different sequences. In some embodiments, the first and second linker comprise the same sequence. In some embodiments, the first and second linker comprise the sequence set forth in SEQ ID NO: 53. In some embodiments, the first and second linker consists of the sequence set forth in SEQ ID NO: 53.

In some embodiments, the heterodimeric protein useful in the methods disclosed herein comprises (i) a first monomer comprising IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein each of said first and second Fc domains independently comprises amino acid substitutions E233P, L234V, L235A, G236del, and S267K, according to EU numbering; and wherein said IL-15 protein comprises an N65D amino acid substitution and one or more amino acid substitutions selected from the group consisting of N4D, D30N, E64Q. The position of the various Fc domain substitutions is in reference to the corresponding position in the wild-type IgG1 Fc domain (SEQ ID NO: 12). The amino acid sequence of the wild-type IgG1 Fc domain (SEQ ID NO: 12) is an exemplary sequence provided for comparison purposes, and the IL-15Rα protein of the heterodimeric protein may comprise additional amino acid alterations (e.g., substitutions, insertions, and deletions) relative to the wild-type IgG1 Fc domain (SEQ ID NO: 12). For example, the Fc domain of the heterodimeric protein may be derived from a different wild-type human IgG1 allele. In some embodiments, the Fc domain of the heterodimeric protein does not comprise any additional amino acid alterations relative to the wild-type IgG1 Fc domain (SEQ ID NO: 12). The amino acid substitutions for the IL-15 protein disclosed herein are relative to wild-type IL-15 (mature form; SEQ ID NO: 1). The amino acid sequence of wild-type IL-15 (mature form; SEQ ID NO: 1) is an exemplary sequence provided for comparison purposes, and the IL-15 protein of the heterodimeric protein may comprise additional amino acid alterations (e.g., substitutions, insertions, and deletions) relative to wild-type IL-15. For example, the IL-15 protein of the heterodimeric protein may be derived from a different wild-type human IL-15 allele. In some embodiments, the IL-15 protein of the heterodimeric protein does not comprise any additional amino acid alterations relative to wild-type IL-15.

The skilled artisan would be able to determine the corresponding substitutions in an Fc domain derived from an IgG2, an IgG3 or an IgG4 Fc domain. For example, the skilled artisan would recognize that residues E233, L234, L235, G236 and A327 are present in Fc domains derived from IgG1 or IgG3 Fc domains. In some embodiments, the position of the various Fc domain substitutions is in reference to the corresponding position in the wild-type IgG3 Fc domain (SEQ ID NO: 14). The amino acid sequence of the wild-type IgG3 Fc domain (SEQ ID NO: 14) is an exemplary sequence provided for comparison purposes, and the IL-15Rα protein of the heterodimeric protein may comprise additional amino acid alterations (e.g., substitutions, insertions, and deletions) relative to the wild-type IgG3 Fc domain (SEQ ID NO: 14). For example, the Fc domain of the heterodimeric protein may be derived from a different wild-type human IgG3 allele. In some embodiments, the Fc domain of the heterodimeric protein does not comprise any additional amino acid alterations relative to the wild-type IgG3 Fc domain (SEQ ID NO: 14). The skilled artisan would recognize, therefore, that each of said first and second Fc domains independently comprises amino acid substitutions E233P, L234V, L235A, G236del, and S267K, according to EU numbering, when the Fc domains are derived from an IgG1 or an IgG3 Fc domain.

The skilled artisan would also recognize that the corresponding residues in a Fc domain derived IgG2 Fc domain are P233, V234, A235 and G327 and that an Fc domain derived from IgG2 lacks a residue corresponding to residue G236. Accordingly, the skilled artisan would recognize that reference to E233P, L234V, L235A G236del and A327G herein is a reference to P233, V234, A235, −236 and no substitution in residue 327, if the Fc domain is derived from an IgG2 Fc domain (i.e., the PVA-sequence present in wild type IgG2). In some embodiments, the position of the various Fc domain substitutions is in reference to the corresponding position in the wild-type IgG2 Fc domain (SEQ ID NO: 13). The amino acid sequence of the wild-type IgG2 Fc domain (SEQ ID NO: 13) is an exemplary sequence provided for comparison purposes, and the IL-15Rα protein of the heterodimeric protein may comprise additional amino acid alterations (e.g., substitutions, insertions, and deletions) relative to the wild-type IgG2 Fc domain (SEQ ID NO: 13). For example, the Fc domain of the heterodimeric protein may be derived from a different wild-type human IgG2 allele. In some embodiments, the Fc domain of the heterodimeric protein does not comprise any additional amino acid alterations relative to the wild-type IgG2 Fc domain (SEQ ID NO: 13). The skilled artisan would recognize, therefore, that each of said first and second Fc domains independently comprises the amino acid substitution S267K, according to EU numbering, when the Fc domains are derived from an IgG2 Fc domain.

The skilled artisan would also recognize that in a Fc domain derived from an IgG4 residue 234 is a phenylalanine and residue 327 is a glycine. Accordingly, the skilled artisan would recognize that reference to L234 herein (e.g., L234V) and A327 (e.g., A327G) is a reference to F234 (e.g., F234V) and no substitution in residue 327, respectively, if the Fc domain is derived from an IgG4 Fc domain. In some embodiments, the position of the various Fc domain substitutions is in reference to the corresponding position in the wild-type IgG4 Fc domain (SEQ ID NO: 15). The amino acid sequence of the wild-type IgG4 Fc domain (SEQ ID NO: 15) is an exemplary sequence provided for comparison purposes, and the IL-15Rα protein of the heterodimeric protein may comprise additional amino acid alterations (e.g., substitutions, insertions, and deletions) relative to the wild-type IgG4 Fc domain (SEQ ID NO: 15). For example, the Fc domain of the heterodimeric protein may be derived from a different wild-type human IgG4 allele. In some embodiments, the Fc domain of the heterodimeric protein does not comprise any additional amino acid alterations relative to the wild-type IgG4 Fc domain (SEQ ID NO: 15). The skilled artisan would recognize, therefore, that each of said first and second Fc domains independently comprises amino acid substitutions E233P, F234V, L235A, G236del, and S267K, according to EU numbering, when the Fc domains are derived from an IgG4 Fc domain.

In some embodiments, the first Fc domain and/or the second Fc domain are independently engineered to further prolong systemic exposure and increase half-life through enhanced FcRn binding at a lower pH (6.0). In some embodiments, additional engineering on the Fc region makes the heterodimeric protein of the disclosure effectorless (i.e. abolish the binding to Fcγ receptors) and eliminates antibody-mediated CL of T cells and NK cells. In some embodiments, the first and/or second Fc domain are independently engineered to encourage heterodimerization formation over homodimerization formation. In some embodiments, the first and/or second Fc domain are independently engineered to have improved PK. In some embodiments, the first and/or second Fc domain are independently engineered to allow purification of homodimers away from heterodimers by increasing the pI difference between the two monomers. In certain embodiments, the Fc variant domain may further comprise a molecule or sequence that lacks one or more native Fc amino acid residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a neonatal receptor, (7) antibody-dependent cell-mediated cytotoxicity (ADCC), or (8) antibody dependent cellular phagocytosis (ADCP). Fc variants are described in further detail hereinafter.

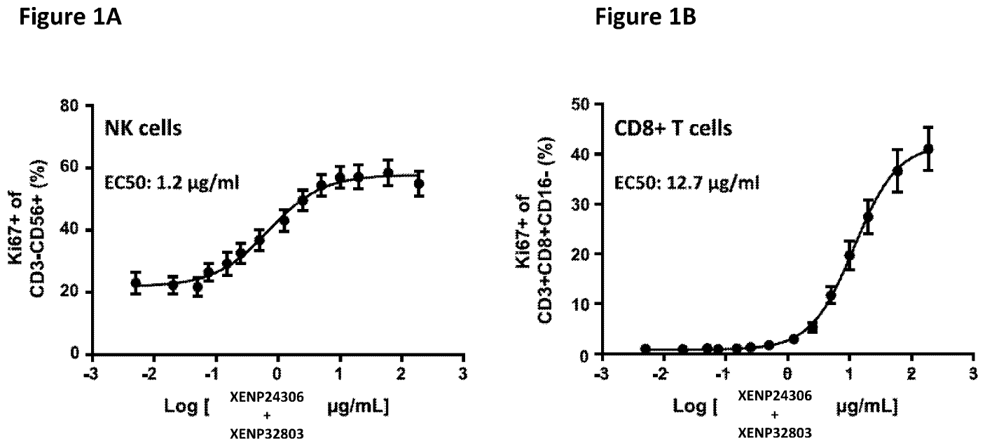

In some embodiments, the first or second Fc domain of the present disclosure may comprise “skew” variants (e.g., a set of amino acid substitutions as shown in FIGS. 1A-1C of U.S. Pat. No. 10,259,887; all of which are herein incorporated by reference in its entirety). Skew variants encourage heterodimerization formation over homodimerization formation. In some embodiments, the skew variants are selected from the group consisting of S364K/E357Q (on the first Fc domain): L368D/K370S (on the second Fc domain); L368D/K370S:S364K; L368E/K370S:S364K; T411E/K360E/Q362E:D401K; L368D/K370S: S364K/E357L, K370S: S364K/E357Q, T366S/L368A/Y407V: T366W and T366S/L368A/Y407V/Y349C: T366W/S354C, according to EU numbering. In some embodiments, said first Fc domain further comprises amino acid substitutions L368D and K370S and said second Fc domain further comprises amino acid substitutions S364K and E357Q, according to EU numbering. In some embodiments, said first Fc domain further comprises amino acid substitutions S364K and E357Q and said second Fc domain further comprises amino acid substitutions L368D and K370S, according to EU numbering.

Alternative methods of generating heterodimeric proteins (e.g., bispecific antibodies or heterodimeric Fc-fusion proteins) are known in the art, including but not limited to “knobs-into-holes” technology and DuoBody® technology. See, e.g., Liu et al. “Fc Engineering for Developing Therapeutic Bispecific Antibodies and Novel Scaffolds,” Front. Immunol. 2017 vol. 8, article 38; Ridgway et al. “‘Knobs-into-holes’engineering of antibody CH3 domains for heavy chain heterodimerization,” Protein Eng. 1996, vol. 9(7): 617-21; Atwell et al. “Stable heterodimers from remodeling the domain interface of a homodimer using a phage display library,” J Mol Biol. 1997 vol. 270:26-35; Merchant et al. “An efficient route to human bispecific IgG,” Nat Biotechnol. 1998 vol 16:677-81; U.S. Pat. No. 8,216,805; Gramer et al. “Production of stable bispecific IgG1 by controlled Fab-arm exchange”, mAbs 5(6): 962-973 (2013); Labrijn et al. “Efficient generation of stable bispecific IgG1 by controlled Fab-arm exchange” PNAS 110(13): 5145-5150 (2013); Labrijn et al. “Controlled Fab-arm exchange for the generation of stable bispecific IgG1” Nature Protocols 9(10): 2450-63 (2014); Labrijn et al. “Efficient Generation of Bispecific Murine Antibodies for Pre-Clinical Investigations in Syngeneic Rodent Models” Scientific Reports 7(1): 1-14 (2017); and van den Bremer et al. “Cysteine-SILAC Mass Spectrometry Enabling the Identification and Quantitation of Scrambled Interchain Disulfide Bonds: Preservation of Native Heavy-Light Chain Pairing in Bispecific IgGs Generated by Controlled Fab-arm Exchange” Analytical Chemistry 89(20), 10873-10882 (2017), each of which is hereby incorporated by reference in its entirety. Any such technologies may be used to generate the heterodimeric proteins of the disclosure.

In some embodiments, the first Fc domain further comprises amino acid substitutions selected from the group consisting of Q295E, N384D, Q418E and N421D, or a combination thereof, according to EU numbering. In some embodiments, the second Fc domain further comprises any one of amino acid substitutions selected from the group consisting of Q295E, N384D, Q418E and N421D, or a combination thereof, according to EU numbering. In some embodiments, the first and second Fc domains each further comprise any one of amino acid substitutions selected from the group consisting of Q295E, N384D, Q418E and N421D, or a combination thereof, according to EU numbering. In some embodiments, said first Fc domain further comprises amino acid substitutions Q295E, N384D, Q418E and N421D, according to EU numbering. In some embodiments, said second Fc domain further comprises amino acid substitutions Q295E, N384D, Q418E and N421D, according to EU numbering. In some embodiments, said first and second Fc domains further comprise amino acid substitutions Q295E, N384D, Q418E and N421D, according to EU numbering.

In some embodiments, the first Fc domain does not comprise a free Cysteine at position 220, according to EU numbering. In some embodiments, the first Fc domain comprises the amino acid substitution C220S, according to EU numbering. In some embodiments, the second Fc domain does not comprise a free Cysteine at position 220, according to EU numbering. In some embodiments, the second Fc domain comprises the amino acid substitution C220S, according to EU numbering. In some embodiments, the first and second Fc domains do not comprise a free Cysteine at position 220, according to EU numbering. In some embodiments, the first and second Fc domains comprise the amino acid substitution C220S, according to EU numbering.

In some embodiments, the first or the second Fc domain of the present disclosure may include amino acid substitutions for improved PK (Xtend substitutions). In some embodiments, the first and/or second Fc domains of the present disclosure independently comprise amino acid substitutions M428L and/or N434S, according to EU numbering. In some embodiments, the first Fc domain comprises the amino acid substitution M428L or N434S. In some embodiments, the first Fc domain comprises amino acid substitutions M428L and N434S. In some embodiments, the first Fc domain comprises the amino acid substitution M428L. In some embodiments, the first Fc domain comprises the amino acid substitution N434S. In some embodiments, the second Fc domain comprises the amino acid substitution M428L or N434S. In some embodiments, the second Fc domain comprises amino acid substitutions M428L and N434S. In some embodiments, the second Fc domain comprises the amino acid substitution M428L. In some embodiments, the second Fc domain comprises the amino acid substitution N434S. In some embodiments, the first and second Fc domains each comprise the amino acid substitution M428L. In some embodiments, the first and second Fc domains each comprise the amino acid substitution N434S. In some embodiments, the first and second Fc domains each comprise amino acid substitutions M428L and N434S.

In some embodiments, said first and/or second Fc domain further comprises amino acid substitution K246T, according to EU numbering. In some embodiments, the first Fc domain further comprises amino acid substitution K246T, according to EU numbering. In some embodiments, the second Fc domain further comprises amino acid substitution K246T, according to EU numbering. When the K246T substitution appears in the second Fc domain, it may also be referred to as a K100T mutation based on the amino acid numbering of the second monomer (see, e.g., SEQ ID NO: 10 and 16). In some embodiments, the first and second Fc domains further comprise amino acid substitution K246T, according to EU numbering.

In some embodiments, the first Fc domain of the heterodimeric protein comprises the sequence set forth in SEQ ID NO: 6. In some embodiments, the second Fc domain of the heterodimeric protein comprises the sequence set forth in SEQ ID NO: 7. In some embodiments, the second Fc domain of the heterodimeric protein comprises the sequence set forth in SEQ ID NO: 8.

In some embodiments, any one of the amino acid substitutions of the Fe variant domains described herein are on one of the monomers or on both monomers (e.g., on the first Fc domain; on the second Fc domain or on both Fc domains).

In some embodiments, the Fc domain of the first monomer is derived from IgG1, IgG2, IgG3, or IgG4. In some embodiments, the Fc domain of the first monomer is derived from IgG1. In some embodiments, the Fc domain of the first monomer is derived from IgG2. In some embodiments, the Fc domain of the first monomer is derived from IgG3. In some embodiments, the Fc domain of the first monomer is derived from IgG4. In some embodiments, the Fc domain of the second monomer is derived from IgG1, IgG2, IgG3, or IgG4. In some embodiments, the Fc domain of the second monomer is derived from IgG1. In some embodiments, the Fc domain of the second monomer is derived from IgG2. In some embodiments, the Fc domain of the second monomer is derived from IgG3. In some embodiments, the Fc domain of the second monomer is derived from IgG4.

In some embodiments, said first Fc domain comprises the following amino acid substitutions: C220S, E233P, L234V, L235A, G236del, S267K, L368D, K370S, M428L and N434S, according to EU numbering. In some embodiments, said second Fc domain comprises the following amino acid substitutions, according to EU numbering: C220S, E233P, L234V, L235A, G236del, S267K, S364K, E357Q, M428L and N434S. In some embodiments, said second Fc domain comprises the following amino acid substitutions: C220S, E233P, L234V, L235A, G236del, S267K, L368D, K370S, M428L and N434S, according to EU numbering. In some embodiments, said first Fc domain comprises the following amino acid substitutions: C220S, E233P, L234V, L235A, G236del, S267K, S364K, E357Q, M428L and N434S, according to EU numbering. In some embodiments, the first Fc domain does not comprise any additional amino acid alterations compared to a wild-type IgG Fc domain. In some embodiments, the first Fc domain does not comprise any additional amino acid alterations compared to a wild-type IgG1 Fc domain. In some embodiments, the first Fc domain does not comprise any additional amino acid alterations compared to SEQ ID NO: 12. In some embodiments, the second Fc domain does not comprise any additional amino acid alterations compared to a wild-type IgG Fc domain. In some embodiments, the second Fc domain does not comprise any additional amino acid alterations compared to a wild-type IgG1 Fc domain. In some embodiments, the second Fc domain does not comprise any additional amino acid alterations compared to SEQ ID NO: 12.

In some embodiments, each of said first and second Fc domains independently comprises an additional set of amino acid substitutions selected from the group consisting of G236R, S239K, L328R, and A327G, according to EU numbering.

In some embodiments, the heterodimeric protein comprises (i) a first monomer comprising an IL-15 protein and a first Fc domain, wherein said IL-15 protein is covalently attached to the N-terminus of said first Fc domain and (ii) a second monomer comprising a wild type sushi domain of IL-15Rα protein and a second Fc domain, wherein said sushi domain of IL-15Rα protein is covalently attached to the N-terminus of said second Fc domain; wherein the first Fc domain comprises amino acid substitutions C220S, E233P, L234V, L235A, G236del, S267K, Q295E, L368D, K370S, N384D, Q418E, N421D, M428L, and N434S, and wherein the second Fc domain comprises amino acid substitutions C220S, E233P, L234V, L235A, G236del, S267K, E357Q, S364K, M428L, and N434S, according to EU numbering; and wherein said IL-15 protein comprises amino acid substitutions D30N, E64Q and N65D, compared to a wild-type IL-15 protein (SEQ ID NO:1).

In some embodiments, the heterodimeric protein comprises (i) a first monomer comprising IL-15 protein and a first Fc domain, wherein said IL-15 protein is covalently attached to the N-terminus of said first Fc domain and (ii) a second monomer comprising a wild type sushi domain of IL-15Rα protein and a second Fc domain, wherein said sushi domain of IL-15Rα protein is covalently attached to the N-terminus of said second Fc domain; wherein the first Fc domain comprises amino acid substitutions C220S, E233P, L234V, L235A, G236del, S267K, Q295E, E357Q, S364K, N384D, Q418E, N421D, M428L, and N434S; and wherein the second Fc domain comprises amino acid substitutions C220S, E233P, L234V, L235A, G236del, S267K, L368D, K370S, M428L, and N434S, according to EU numbering; and wherein said IL-15 protein comprises amino acid substitutions D30N, E64Q and N65D, compared to a wild-type IL-15 protein (SEQ ID NO:1).

In some embodiments, the heterodimeric protein comprises (i) a first monomer comprising IL-15 protein and a first Fc domain, wherein said IL-15 protein is covalently attached to the N-terminus of said first Fc domain and (ii) a second monomer comprising a wild type sushi domain of IL-15Rα protein and a second Fc domain, wherein said sushi domain of IL-15Rα protein is covalently attached to the N-terminus of said second Fc domain; wherein the first Fc domain comprises amino acid substitutions C220S, E233P, L234V, L235A, G236del, S267K, Q295E, L368D, K370S, N384D, Q418E, N421D, M428L, and N434S, and wherein the second Fc domain comprises amino acid substitutions C220S, E233P, L234V, L235A, G236del, K246T, S267K, E357Q, S364K, M428L, and N434S, according to EU numbering; and wherein said IL-15 protein comprises amino acid substitutions D30N, E64Q and N65D, compared to a wild-type IL-15 protein (SEQ ID NO:1).

In some embodiments, the heterodimeric protein comprises (i) a first monomer comprising IL-15 protein and a first Fc domain, wherein said IL-15 protein is covalently attached to the N-terminus of said first Fc domain and (ii) a second monomer comprising a wild type sushi domain of IL-15Rα protein and a second Fc domain, wherein said sushi domain of IL-15Rα protein is covalently attached to the N-terminus of said second Fc domain; wherein the first Fc domain comprises amino acid substitutions C220S, E233P, L234V, L235A, G236del, S267K, Q295E, E357Q, S364K, N384D, Q418E, N421D, M428L, and N434S; and wherein the second Fc domain comprises amino acid substitutions C220S, E233P, L234V, L235A, G236del, K246T, S267K, L368D, K370S, M428L, and N434S, according to EU numbering; and wherein said IL-15 protein comprises amino acid substitutions D30N, E64Q and N65D, compared to a wild-type IL-15 protein (SEQ ID NO:1).

In some embodiments, the first monomer comprises the amino acid sequence set forth in SEQ ID NO: 9, and the second monomer comprises the amino acid sequence set forth in SEQ ID NO: 10. In some embodiments, the first monomer comprises the amino acid sequence set forth in SEQ ID NO: 9, and the second monomer comprises the amino acid sequence set forth in SEQ ID NO: 16.

In some embodiments, the first monomer comprises (1) IL-15 and (2) a first Fc domain that comprises the sequence set forth in SEQ ID NO: 6. In some embodiments, the second monomer comprises (1) IL-15Rα and (2) a second Fc domain that comprises the sequence set forth in SEQ ID NO: 7.

In some embodiments, the amino acid substitutions present in the heterodimeric protein are disclosed in U.S. Patent Publication US 2018/0118805 and are incorporated herein by reference in its entirety.

The sequences referenced herein are provided in Table 1, infra. It is known in the art that during the processing and expression of Fc-containing proteins that the C-terminal lysine may be cleaved (also known in the art as C-terminal lysine clipping). Accordingly, for each sequence disclosed herein that contains a C-terminal lysine, the corresponding sequence without the C-terminal lysine (i.e., the C-terminal lysine cleavage product) is also contemplated. In some embodiments, the first monomer comprises a C-terminal lysine. In some embodiments, the first monomer lacks a C-terminal lysine. In some embodiments, the second monomer comprises a C-terminal lysine. In some embodiments, the second monomer lacks a C-terminal lysine.

It is also known in the art that the C-terminal cleavage process is imprecise and that additional C-terminal residues are cleaved. Accordingly, for each sequence disclosed herein that contains a C-terminal lysine, the corresponding sequence without the two C-terminal residues is also contemplated. In some embodiments, for each sequence disclosed herein that contains a C-terminal lysine, the corresponding sequence without the three C-terminal residues is also contemplated. In some embodiments, for each sequence disclosed herein that contains a C-terminal lysine, the corresponding sequence without the four C-terminal residues is also contemplated. In some embodiments, for each sequence disclosed herein that contains a C-terminal lysine, the corresponding sequence without the five C-terminal residues is also contemplated. In some embodiments, for each sequence disclosed herein that contains a C-terminal lysine, the corresponding sequence without the six C-terminal residues is also contemplated. In some embodiments, for each sequence disclosed herein that contains a C-terminal lysine, the corresponding sequence without the seven C-terminal residues is also contemplated. In some embodiments, for each sequence disclosed herein that contains a C-terminal lysine, the corresponding sequence without the eight C-terminal residues is also contemplated. In some embodiments, for each sequence disclosed herein that contains a C-terminal lysine, the corresponding sequence without the nine C-terminal residues is also contemplated. In some embodiments, for each sequence disclosed herein that contains a C-terminal lysine, the corresponding sequence without the ten C-terminal residues is also contemplated. In some embodiments, for each sequence disclosed herein that contains a C-terminal lysine, the corresponding sequence without the eleven C-terminal residues is also contemplated. In some embodiments, for each sequence disclosed herein that contains a C-terminal lysine, the corresponding sequence without the twelve C-terminal residues is also contemplated. In some embodiments, for each sequence disclosed herein that contains a C-terminal lysine, the corresponding sequence without the thirteen C-terminal residues is also contemplated. In some embodiments, for each sequence disclosed herein that contains a C-terminal lysine, the corresponding sequence without the fourteen C-terminal residues is also contemplated. In some embodiments, for each sequence disclosed herein that contains a C-terminal lysine, the corresponding sequence without the fifteen C-terminal residues is also contemplated. In some embodiments, the missing C-terminal residues are the result of engineering (e.g., expressing a polynucleotide missing the nucleotide sequences encoding one or more of the C-terminal residues).

TABLE 1
Compilation of amino acid sequences described in the present disclosure.

| | | |
|---|---|---|
| SEQ ID | Wild-type | NWVNVISDLKKIEDLIQSMHIDATLYTESDVHPS |
| NO: 1 | mature or | CKVTAMKCFLLELQVISLESGDASIHDTVENLIIL |
| | truncated IL-15 | ANNSLSSNGNVTESGCKECEELEEKNIKEFLQSF |
| | protein | VHIVQMFINTS |
| SEQ ID | Wild-type full- | MRISKPHLRSISIQCYLCLLLNSHFLTEAGIHVFIL |
| NO: 2 | length IL-15 | GCFSAGLPKTEANWVNVISDLKKIEDLIQSMHID |
| | protein | ATLYTESDVHPSCKVTAMKCFLLELQVISLESGD |
| | | ASIHDTVENLIILANNSLSSNGNVTESGCKECEEL |
| | | EEKNIKEFLQSFVHIVQMFINTS |
| SEQ ID | Wild-type full- | MAPRRARGCRTLGLPALLLLLLLRPPATRGITCPP |
| NO: 3 | length IL-15Rα | PMSVEHADIWVKSYSLYSRERYICNSGFKRKAG |
| | protein | TSSLTECVLNKATNVAHWTTPSLKCIRDPALVHQ |
| | | RPAPPSTVTTAGVTPQPESLSPSGKEPAASSPSSN |
| | | NTAATTAAIVPGSQLMPSKSPSTGTTEISSHESSH |
| | | GTPSQTTAKNWELTASASHQPPGVYPQGHSDTT |
| | | VAISTSTVLLCGLSAVSLLACYLKSRQTPPLASVE |
| | | MEAMEALPVTWGTSSRDEDLENCSHHL |
| SEQ ID | Sushi domain of | ITCPPPMSVEHADIWVKSYSLYSRERYICNSGFKR |
| NO: 4 | IL-15Rα protein | KAGTSSLTECVLNKATNVAHWTTPSLKCIR |
| SEQ ID | XENP24306 or | NWVNVISDLKKIEDLIQSMHIDATLYTESNVHPS |
| NO: 5 | XENP32803 IL- | CKVTAMKCFLLELQVISLESGDASIHDTVQDLIIL |
| | 15 protein | ANNSLSSNGNVTESGCKECEELEEKNIKEFLQSF |
| | variant | VHIVQMFINTS |
| SEQ ID | XENP24306 or | EPKSSDKTHTCPPCPAPPVAGPSVFLFPPKPKDTL |
| NO: 6 | XENP32803 | MISRTPEVTCVVVDVKHEDPEVKFNWYVDGVE |
| | First IgG1 Fc | VHNAKTKPREEEYNSTYRVVSVLTVLHQDWLN |
| | domain (IL-15 | GKEYKCKVSNKALPAPIEKTISKAKGQPREPQVY |
| | monomer) | TLPPSREEMTKNQVSLTCDVSGFYPSDIAVEWES |
| | | DGQPENNYKTTPPVLDSDGSFFLYSKLTVDKSR |
| | | WEQGDVFSCSVLHEALHSHYTQKSLSLSPGK |
| SEQ ID | XENP24306 | EPKSSDKTHTCPPCPAPPVAGPSVFLFPPKPKDTL |
| NO: 7 | Second IgG1 Fc | MISRTPEVTCVVVDVKHEDPEVKFNWYVDGVE |
| | domain (IL- | VHNAKTKPREEQYNSTYRVVSVLTVLHQDWLN |
| | 15Rα monomer) | GKEYKCKVSNKALPAPIEKTISKAKGQPREPQVY |
| | | TLPPSREQMTKNQVKLTCLVKGFYPSDIAVEWES |
| | | NGQPENNYKTTPPVLDSDGSFFLYSKLTVDKSR |
| | | WQQGNVFSCSVLHEALHSHYTQKSLSLSPGK |
| SEQ ID | XENP32803 | EPKSSDKTHTCPPCPAPPVAGPSVFLFPPTPKDTL |
| NO: 8 | Second IgG1 Fc | MISRTPEVTCVVVDVKHEDPEVKFNWYVDGVE |
| | domain (IL- | VHNAKTKPREEQYNSTYRVVSVLTVLHQDWLN |
| | 15Rα monomer) | GKEYKCKVSNKALPAPIEKTISKAKGQPREPQVY |
| | | TLPPSREQMTKNQVKLTCLVKGFYPSDIAVEWES |
| | | NGQPENNYKTTPPVLDSDGSFFLYSKLTVDKSR |
| | | WQQGNVFSCSVLHEALHSHYTQKSLSLSPGK |
| SEQ ID | XENP24306 or | NWVNVISDLKKIEDLIQSMHIDATLYTESNVHPS |
| NO: 9 | XENP32803 | CKVTAMKCFLLELQVISLESGDASIHDTVQDLIIL |
| | First monomer | ANNSLSSNGNVTESGCKECEELEEKNIKEFLQSF |
| | (IL-15-first Fc | VHIVQMFINTSGGGGSEPKSSDKTHTCPPCPAPPV |
| | domain | AGPSVFLFPPKPKDTLMISRTPEVTCVVVDVKHE |
| | monomer) | DPEVKFNWYVDGVEVHNAKTKPREEEYNSTYR |
| | | VVSVLTVLHQDWLNGKEYKCKVSNKALPAPIEK |
| | | TISKAKGQPREPQVYTLPPSREEMTKNQVSLTCD |
| | | VSGFYPSDIAVEWESDGQPENNYKTTPPVLDSDG |
| | | SFFLYSKLTVDKSRWEQGDVFSCSVLHEALHSH |
| | | YTQKSLSLSPGK |
| SEQ ID | XENP24306 | ITCPPPMSVEHADIWVKSYSLYSRERYICNSGFKR |
| NO: 10 | Second monomer | KAGTSSLTECVLNKATNVAHWTTPSLKCIRGGG |
| | (IL-15Rα-second | GSEPKSSDKTHTCPPCPAPPVAGPSVFLFPPKPKD |
| | Fc domain | TLMISRTPEVTCVVVDVKHEDPEVKFNWYVDGV |
| | monomer) | EVHNAKTKPREEQYNSTYRVVSVLTVLHQDWL |
| | | NGKEYKCKVSNKALPAPIEKTISKAKGQPREPQV |
| | | YTLPPSREQMTKNQVKLTCLVKGFYPSDIAVEW |
| | | ESNGQPENNYKTTPPVLDSDGSFFLYSKLTVDKS |
| | | RWQQGNVFSCSVLHEALHSHYTQKSLSLSPGK |
| SEQ ID | XENP22853 | NWVNVISDLKKIEDLIQSMHIDATLYTESDVHPS |
| NO: 11 | Wild-type first | CKVTAMKCFLLELQVISLESGDASIHDTVENLIIL |
| | monomer (IL-15- | ANNSLSSNGNVTESGCKECEELEEKNIKEFLQSF |
| | first Fc domain | VHIVQMFINTSGGGGSEPKSSDKTHTCPPCPAPPV |
| | monomer) | AGPSVFLFPPKPKDTLMISRTPEVTCVVVDVKHE |
| | | DPEVKFNWYVDGVEVHNAKTKPREEEYNSTYR |
| | | VVSVLTVLHQDWLNGKEYKCKVSNKALPAPIEK |
| | | TISKAKGQPREPQVYTLPPSREEMTKNQVSLTCD |
| | | VSGFYPSDIAVEWESDGQPENNYKTTPPVLDSDG |
| | | SFFLYSKLTVDKSRWEQGDVFSCSVLHEALHSH |
| | | YTQKSLSLSPGK |
| SEQ ID | Unmodified Fc | EPKSCDKTHTCPPCPAPELLGGPSVFLFPPKPKDT |
| NO: 12 | IgG1 domain | LMISRTPEVTCVVVDVSHEDPEVKFNWYVDGVE |
| | (allele 3; | VHNAKTKPREEQYNSTYRVVSVLTVLHQDWLN |
| | Y14737) | GKEYKCKVSNKALPAPIEKTISKAKGQPREPQVY |
| | | TLPPSREEMTKNQVSLTCLVKGFYPSDIAVEWES |
| | | NGQPENNYKTTPPVLDSDGSFFLYSKLTVDKSR |
| | | WQQGNVFSCSVMHEALHNHYTQKSLSLSPGK |
| SEQ ID | Unmodified Fc | ERKCCVECPPCPAPPVAGPSVFLFPPKPKDTLMIS |
| NO: 13 | IgG2 domain | RTPEVTCVVVDVSHEDPEVQFNWYVDGVEVHN |
| | (allele 1; J00230/ | AKTKPREEQFNSTFRVVSVLTVVHQDWLNGKEY |
| | AH005273) | KCKVSNKGLPAPIEKTISKTKGQPREPQVYTLPPS |
| | | REEMTKNQVSLTCLVKGFYPSDIAVEWESNGQP |
| | | ENNYKTTPPMLDSDGSFFLYSKLTVDKSRWQQG |
| | | NVFSCSVMHEALHNHYTQKSLSLSPGK |
| SEQ ID | Unmodified Fc | EPKSCDTPPPCPRCPAPELLGGPSVFLFPPKPKDT |
| NO: 14 | IgG3 domain | LMISRTPEVTCVVVDVSHEDPEVQFKWYVDGVE |
| | (allele 8; | VHNAKTKPREEQYNSTFRVVSVLTVLHQDWLN |
| | AJ390241/ | GKEYKCKVSNKALPAPIEKTISKTKQPREPQVYT |
| | X03604) | LPPSREEMTKNQVSLTCLVKGFYPSDIAVEWESN |
| | | GQPENNYNTTPPMLDSDGSFFLYSKLTVDKSRW |
| | | QQGNIFSCSVMHEALHNRFTQKSLSLSPGK |
| SEQ ID | Unmodified Fc | ESKYGPPCPSCPAPEFLGGPSVFLFPPKPKDTLMI |
| NO: 15 | IgG4 domain | SRTPEVTCVVVDVSQEDPEVQFNWYVDGVEVH |
| | (allele 1; | NAKTKPREEQFNSTYRVVSVLTVLHQDWLNGKE |
| | K01316/ | YKCKVSNKGLPSSIEKTISKAKGQPREPQVYTLPP |
| | AH005273) | SQEEMTKNQVSLTCLVKGFYPSDIAVEWESNGQ |
| | | PENNYKTTPPVLDSDGSFFLYSRLTVDKSRWQEG |
| | | NVFSCSVMHEALHNHYTQKSLSLSLGK |
| SEQ ID | XENP32803 | ITCPPPMSVEHADIWVKSYSLYSRERYICNSGFKR |
| NO: 16 | Second monomer | KAGTSSLTECVLNKATNVAHWTTPSLKCIRGGG |
| | (IL-15Rα | GSEPKSSDKTHTCPPCPAPPVAGPSVFLFPPTPKD |
| | monomer). | TLMISRTPEVTCVVVDVKHEDPEVKFNWYVDGV |
| | | EVHNAKTKPREEQYNSTYRVVSVLTVLHQDWL |
| | | NGKEYKCKVSNKALPAPIEKTISKAKGQPREPQV |
| | | YTLPPSREQMTKNQVKLTCLVKGFYPSDIAVEW |
| | | ESNGQPENNYKTTPPVLDSDGSFFLYSKLTVDKS |
| | | RWQQGNVFSCSVLHEALHSHYTQKSLSLSPGK |

In some embodiments, the heterodimeric protein of the disclosure is selected from the group consisting of XENP20818, XENP20819, XENP21471, XENP21472, XENP21473, XENP21474, XENP21475, XENP21476, XENP21477, XENP21988, XENP21989, XENP21990, XENP21991, XENP21992, XENP22013, XENP22014, XENP22015, XENP22017, XENP22815, XENP22816, XENP22817, XENP22818, XENP22819, XENP22820, XENP22821, XENP22822, XENP22823, XENP22824, XENP22825, XENP22826, XENP22827, XENP22828, XENP22829, XENP22830, XENP22831, XENP22832, XENP22833, XENP22834, XENP23343, XENP23472, XENP23504, XENP23554, XENP23555, XENP23557, XENP23559, XENP23560, XENP23561, XENP24017, XENP24018, XENP24019, XENP24020, XENP24043, XENP24044, XENP24046, XENP24051, XENP24052, XENP24113, XENP24301, XENP24306, XENP24341, and XENP32803 heterodimeric proteins, the sequences of which are disclosed in FIGS. 104A-104AY of U.S. Pat. No. 10,501,543 and are incorporated by reference herein.

In some embodiments, the heterodimeric protein of the disclosure is selected from the group consisting of XENP22822, XENP23504, XENP24045, XENP24306, XENP22821, XENP23343, XENP23557, XENP24113, XENP24051, XENP24341, XENP24052, XENP24301, and XENP32803 heterodimeric proteins, which are described in Table 2 below. The sequences of XENP22822, XENP23504, XENP24045, XENP24306, XENP22821, XENP23343, XENP23557, XENP24113, XENP24051, XENP24341, XENP24052, and XENP24301 are also provided in US 2018/0118805 and are incorporated by reference herein. In some embodiments, the heterodimeric protein of the disclosure is XENP24306. In some embodiments, the heterodimeric protein of the disclosure is XENP32803. In some embodiments, the heterodimeric protein is XENP24306, XENP32803, or a combination thereof. In some embodiments, a combination of two or more (e.g., 2, 3, 4, 5, etc.) heterodimeric proteins of the disclosure are used in the methods disclosed herein. In some embodiments, a combination of two heterodimeric proteins of the disclosure (i.e. a first and a second heterodimeric protein of the disclosure) are used in the methods disclosed herein. In some embodiments, the first heterodimeric protein comprises a first monomer comprising the amino acid sequence set forth in SEQ ID NO: 9, and a second monomer comprising the amino acid sequence set forth in SEQ ID NO: 10; and the second heterodimeric protein comprises a first monomer comprising the amino acid sequence set forth in SEQ ID NO: 9, and a second monomer comprising the amino acid sequence set forth in SEQ ID NO: 16. In some embodiments, a combination of XENP24306 and XENP32803 is used in the methods disclosed herein.

In some embodiments, the XENP24306 protein represents about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 75%, about 70%, about 65%, about 60%, about 55%, about 50%, about 45%, about 40%, about 35%, about 30%, about 25%, about 20%, about 15%, about 10%, or about 5% of the heterodimeric protein in the combination. In some embodiments, the XENP24306 protein represents about 85% of the heterodimeric protein in the combination. In some embodiments, the XENP24306 protein represents about 84% of the heterodimeric protein in the combination. In some embodiments, the XENP24306 protein represents about 83% of the heterodimeric protein in the combination. In some embodiments, the XENP24306 protein represents about 82% of the heterodimeric protein in the combination. In some embodiments, the XENP24306 protein represents about 81% of the heterodimeric protein in the combination. In some embodiments, the XENP24306 protein represents about 80% of the heterodimeric protein in the combination.

In some embodiments, the XENP32803 protein represents about 95%, about 90%, about 85%, about 80%, about 75%, about 70%, about 75%, about 70%, about 65%, about 55%, about 50%, about 45%, about 40%, about 35%, about 30%, about 25%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2% or about 1% of the heterodimeric protein in the combination. In some embodiments, the XENP32803 protein represents about 15% of the heterodimeric protein in the combination. In some embodiments, the XENP32803 protein represents about 16% of the heterodimeric protein in the combination. In some embodiments, the XENP32803 protein represents about 17% of the heterodimeric protein in the combination. In some embodiments, the XENP32803 protein represents about 18% of the heterodimeric protein in the combination. In some embodiments, the XENP32803 protein represents about 19% of the heterodimeric protein in the combination. In some embodiments, the XENP32803 protein represents about 20% of the heterodimeric protein in the combination.

In some embodiments, the XENP24306 protein represents between about 50-100%, about 70-95%, about 80-90%, or about 80-85% of the heterodimeric protein in the combination. In some embodiments of any of the methods disclosed herein, the XENP32803 protein represents between about 1-50%, about 5-30%, about 10-20%, or about 15-20% of the heterodimeric protein in the combination. In some embodiments, the XENP24306 protein represents about 85% of the heterodimeric protein in the combination, and the XENP32803 protein represents about 15% of the heterodimeric protein in the combination. In some embodiments, the XENP24306 protein represents about 84% of the heterodimeric protein in the combination, and the XENP32803 protein represents about 16% of the heterodimeric protein in the combination. In some embodiments, the XENP24306 protein represents about 83% of the heterodimeric protein in the combination, and the XENP32803 protein represents about 17% of the heterodimeric protein in the combination. In some embodiments, the XENP24306 protein represents about 82% of the heterodimeric protein in the combination, and the XENP32803 protein represents about 18% of the heterodimeric protein in the combination. In some embodiments, the XENP24306 protein represents about 81% of the heterodimeric protein in the combination, and the XENP32803 protein represents about 19% of the heterodimeric protein in the combination. In some embodiments, the XENP24306 protein represents about 80% of the heterodimeric protein in the combination, and the XENP32803 protein represents about 20% of the heterodimeric protein in the combination.

In some embodiments, the XENP24306 protein represents 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, 45%, 40%, 35%, 30%, 25%, 20%, 15%, 10%, or 5% of the heterodimeric protein in the combination. In some embodiments, the XENP24306 protein represents 85% of the heterodimeric protein in the combination. In some embodiments, the XENP24306 protein represents 84% of the heterodimeric protein in the combination. In some embodiments, the XENP24306 protein represents 83% of the heterodimeric protein in the combination. In some embodiments, the XENP24306 protein represents 82% of the heterodimeric protein in the combination. In some embodiments, the XENP24306 protein represents 81% of the heterodimeric protein in the combination. In some embodiments, the XENP24306 protein represents 80% of the heterodimeric protein in the combination.

In some embodiments, the XENP32803 protein represents 95%, 90%, 85%, 80%, 75%, 70%, 75%, 70%, 65%, 55%, 50%, 45%, 40%, 35%, 30%, 25%, 20%, 19%, 18%, 17%, 16%, 15%, 14%, 13%, 12%, 11%, 10% 9%, 8%, 7%, 6%, 5%, 4%, 3%, 2% or 1% of the heterodimeric protein in the combination. In some embodiments, the XENP32803 protein represents 15% of the heterodimeric protein in the combination. In some embodiments, the XENP32803 protein represents 16% of the heterodimeric protein in the combination. In some embodiments, the XENP32803 protein represents 17% of the heterodimeric protein in the combination. In some embodiments, the XENP32803 protein represents 18% of the heterodimeric protein in the combination. In some embodiments, the XENP32803 protein represents 19% of the heterodimeric protein in the combination. In some embodiments, the XENP32803 protein represents 20% of the heterodimeric protein in the combination.

In some embodiments, the XENP24306 protein represents between 50-100%, 70-95%, 80-90%, or 80-85% of the heterodimeric protein in the combination. In some embodiments of any of the methods disclosed herein, the XENP32803 protein represents between 1-50%, 5-30%, 10-20%, or 15-20% of the heterodimeric protein in the combination. In some embodiments, the XENP24306 protein represents 85% of the heterodimeric protein of the heterodimeric protein in the combination, and the XENP32803 protein represents 15% of the heterodimeric protein in the combination. In some embodiments, the XENP24306 protein represents 84% of the heterodimeric protein in the combination, and the XENP32803 protein represents 16% of the heterodimeric protein in the combination. In some embodiments, the XENP24306 protein represents 83% of the heterodimeric protein in the combination, and the XENP32803 protein represents 17% of the heterodimeric protein in the combination. In some embodiments, the XENP24306 protein represents 82% of the heterodimeric protein in the combination, and the XENP32803 protein represents 18% of the heterodimeric protein in the combination. In some embodiments, the XENP24306 protein represents 810 of the heterodimeric protein in the combination, and the XENP32803 protein represents 1900 of the heterodimeric protein in the combination. In some embodiments, the XENP24306 protein represents 80% a of the heterodimeric protein in the combination, and the XENP32803 protein represents 20% of the heterodimeric protein in the combination.

TABLE 2

| | |
|---|---|
| XENP22821 | Monomer 1 (IL-15 (N65D)-first Fc domain). SEQ ID NO: 17 |
| | NWVNVISDLKKIEDLIQSMHIDATLYTESDVHPSCKVTAMKCF |
| | LLELQVISLESGDASIHDTVEDLIILANNSLSSNGNVTESGCKEC |
| | EELEEKNIKEFLQSFVHIVQMFINTSGGGGSEPKSSDKTHTCPPC |
| | PAPPVAGPSVFLFPPKPKDTLMISRTPEVTCVVVDVKHEDPEVK |
| | FNWYVDGVEVHNAKTKPREEEYNSTYRVVSVLTVLHQDWLN |
| | GKEYKCKVSNKALPAPIEKTISKAKGQPREPQVYTLPPSREEMT |
| | KNQVSLTCDVSGFYPSDIAVEWESDGQPENNYKTTPPVLDSDG |
| | SFFLYSKLTVDKSRWEQGDVFSCSVMHEALHNHYTQKSLSLSP |
| | GK |
| | Monomer 2 (IL-15Rα-second Fc domain), SEQ ID NO: 18 |
| | ITCPPPMSVEHADIWVKSYSLYSRERYICNSGFKRKAGTSSLTE |
| | CVLNKATNVAHWTTPSLKCIRGGGGSEPKSSDKTHTCPPCPAPP |
| | VAGPSVFLFPPKPKDTLMISRTPEVTCVVVDVKHEDPEVKFNW |
| | YVDGVEVHNAKTKPREEQYNSTYRVVSVLTVLHQDWLNGKE |
| | YKCKVSNKALPAPIEKTISKAKGQPREPQVYTLPPSREQMTKNQ |
| | VKLTCLVKGFYPSDIAVEWESNGQPENNYKTTPPVLDSDGSFFL |
| | YSKLTVDKSRWQQGNVFSCSVMHEALHNHYTQKSLSLSPGK |
| XENP22822 | Monomer 1 (IL-15 (Q108E)-first Fc domain). SEQ ID NO: 19 |
| | NWVNVISDLKKIEDLIQSMHIDATLYTESDVHPSCKVTAMKCF |
| | LLELQVISLESGDASIHDTVENLIILANNSLSSNGNVTESGCKEC |
| | EELEEKNIKEFLQSFVHIVEMFINTSGGGGSEPKSSDKTHTCPPC |
| | PAPPVAGPSVFLFPPKPKDTLMISRTPEVTCVVVDVKHEDPEVK |
| | FNWYVDGVEVHNAKTKPREEEYNSTYRVVSVLTVLHQDWLN |
| | GKEYKCKVSNKALPAPIEKTISKAKGQPREPQVYTLPPSREEMT |
| | KNQVSLTCDVSGFYPSDIAVEWESDGQPENNYKTTPPVLDSDG |
| | SFFLYSKLTVDKSRWEQGDVFSCSVMHEALHNHYTQKSLSLSP |
| | GK |
| | Monomer 2 (IL-15Rα-second Fc domain). SEQ ID NO: 20 |
| | ITCPPPMSVEHADIWVKSYSLYSRERYICNSGFKRKAGTSSLTE |
| | CVLNKATNVAHWTTPSLKCIRGGGGSEPKSSDKTHTCPPCPAPP |
| | VAGPSVFLFPPKPKDTLMISRTPEVTCVVVDVKHEDPEVKFNW |
| | YVDGVEVHNAKTKPREEQYNSTYRVVSVLTVLHQDWLNGKE |
| | YKCKVSNKALPAPIEKTISKAKGQPREPQVYTLPPSREQMTKNQ |
| | VKLTCLVKGFYPSDIAVEWESNGQPENNYKTTPPVLDSDGSFFL |
| | YSKLTVDKSRWQQGNVFSCSVMHEALHNHYTQKSLSLSPGK |
| XENP23557 | Monomer 1 (IL-15 (N4D/N65D)-first Fc domain). SEQ ID NO: 21 |
| | NWVDVISDLKKIEDLIQSMHIDATLYTESDVHPSCKVTAMKCF |
| | LLELQVISLESGDASIHDTVEDLIILANNSLSSNGNVTESGCKEC |
| | EELEEKNIKEFLQSFVHIVQMFINTSGGGGSEPKSSDKTHTCPPC |
| | PAPPVAGPSVFLFPPKPKDTLMISRTPEVTCVVVDVKHEDPEVK |
| | FNWYVDGVEVHNAKTKPREEEYNSTYRVVSVLTVLHQDWLN |
| | GKEYKCKVSNKALPAPIEKTISKAKGQPREPQVYTLPPSREEMT |
| | KNQVSLTCDVSGFYPSDIAVEWESDGQPENNYKTTPPVLDSDG |
| | SFFLYSKLTVDKSRWEQGDVFSCSVMHEALHNHYTQKSLSLSP |
| | GK |
| | Monomer 2 (IL-15Rα-second Fc domain). SEQ ID NO: 22 |
| | ITCPPPMSVEHADIWVKSYSLYSRERYICNSGFKRKAGTSSLTE |
| | CVLNKATNVAHWTTPSLKCIRGGGGSEPKSSDKTHTCPPCPAP |
| | PVAGPSVFLFPPKPKDTLMISRTPEVTCVVVDVKHEDPEVKFN |
| | WYVDGVEVHNAKTKPREEQYNSTYRVVSVLTVLHQDWLNGK |
| | EYKCKVSNKALPAPIEKTISKAKGQPREPQVYTLPPSREQMTKN |
| | QVKLTCLVKGFYPSDIAVEWESNGQPENNYKTTPPVLDSDGSF |
| | FLYSKLTVDKSRWQQGNVFSCSVMHEALHNHYTQKSLSLSPG |
| | K |
| XENP24045 | Monomer 1 (IL-15 (D30N/E64Q/N65D)-first Fc domain). SEQ ID NO: |
| | 23 |
| | NWVNVISDLKKIEDLIQSMHIDATLYTESNVHPSCKVTAMKCF |
| | LLELQVISLESGDASIHDTVQDLIILANNSLSSNGNVTESGCKEC |
| | EELEEKNIKEFLQSFVHIVQMFINTSGGGGSEPKSSDKTHTCPPC |
| | PAPPVAGPSVFLFPPKPKDTLMISRTPEVTCVVVDVKHEDPEVK |
| | FNWYVDGVEVHNAKTKPREEEYNSTYRVVSVLTVLHQDWLN |
| | GKEYKCKVSNKALPAPIEKTISKAKGQPREPQVYTLPPSREEMT |
| | KNQVSLTCDVSGFYPSDIAVEWESDGQPENNYKTTPPVLDSDG |
| | SFFLYSKLTVDKSRWEQGDVFSCSVMHEALHNHYTQKSLSLS |
| | PGK |
| | Monomer 2 (IL-15Rα-second Fc domain), SEQ ID NO: 24 |
| | ITCPPPMSVEHADIWVKSYSLYSRERYICNSGFKRKAGTSSLTE |
| | CVLNKATNVAHWTTPSLKCIRGGGGSEPKSSDKTHTCPPCPAP |
| | PVAGPSVFLFPPKPKDTLMISRTPEVTCVVVDVKHEDPEVKFN |
| | WYVDGVEVHNAKTKPREEQYNSTYRVVSVLTVLHQDWLNGK |
| | EYKCKVSNKALPAPIEKTISKAKGQPREPQVYTLPPSREQMTKN |
| | QVKLTCLVKGFYPSDIAVEWESNGQPENNYKTTPPVLDSDGSF |
| | FLYSKLTVDKSRWQQGNVFSCSVMHEALHNHYTQKSLSLSPG |
| | K |
| XENP24051 | Monomer 1 (IL-15 (NID/N65D)-first Fc domain). SEQ ID NO: 25 |
| | DWVNVISDLKKIEDLIQSMHIDATLYTESDVHPSCKVTAMKCF |
| | LLELQVISLESGDASIHDTVEDLIILANNSLSSNGNVTESGCKEC |
| | EELEEKNIKEFLQSFVHIVQMFINTSEPKSSDKTHTCPPCPAPPV |
| | AGPSVFLFPPKPKDTLMISRTPEVTCVVVDVKHEDPEVKFNWY |
| | VDGVEVHNAKTKPREEEYNSTYRVVSVLTVLHQDWLNGKEY |
| | KCKVSNKALPAPIEKTISKAKGQPREPQVYTLPPSREEMTKNQV |
| | SLTCDVSGFYPSDIAVEWESDGQPENNYKTTPPVLDSDGSFFLY |
| | SKLTVDKSRWEQGDVFSCSVMHEALHNHYTQKSLSLSPGK |
| | Monomer 2 (IL-15Rα-second Fc domain). SEQ ID NO: 26 |
| | ITCPPPMSVEHADIWVKSYSLYSRERYICNSGFKRKAGTSSLTE |
| | CVLNKATNVAHWTTPSLKCIREPKSSDKTHTCPPCPAPPVAGPS |
| | VFLFPPKPKDTLMISRTPEVTCVVVDVKHEDPEVKFNWYVDGV |
| | EVHNAKTKPREEQYNSTYRVVSVLTVLHQDWLNGKEYKCKVS |
| | NKALPAPIEKTISKAKGQPREPQVYTLPPSREQMTKNQVKLTCL |
| | VKGFYPSDIAVEWESNGQPENNYKTTPPVLDSDGSFFLYSKLTV |
| | DKSRWQQGNVFSCSVMHEALHNHYTQKSLSLSPGK |
| XENP24052 | Monomer 1 (IL-15 (N4D/N65D)-first Fc domain). SEQ ID NO: 27 |
| | NWVDVISDLKKIEDLIQSMHIDATLYTESDVHPSCKVTAMKCF |
| | LLELQVISLESGDASIHDTVEDLIILANNSLSSNGNVTESGCKEC |
| | EELEEKNIKEFLQSFVHIVQMFINTSEPKSSDKTHTCPPCPAPPV |
| | AGPSVFLFPPKPKDTLMISRTPEVTCVVVDVKHEDPEVKFNWY |
| | VDGVEVHNAKTKPREEEYNSTYRVVSVLTVLHQDWLNGKEY |
| | KCKVSNKALPAPIEKTISKAKGQPREPQVYTLPPSREEMTKNQV |
| | SLTCDVSGFYPSDIAVEWESDGQPENNYKTTPPVLDSDGSFFLY |
| | SKLTVDKSRWEQGDVFSCSVMHEALHNHYTQKSLSLSPGK |
| | Monomer 2 (IL-15Rα-second Fc domain), SEQ ID NO: 28 |
| | ITCPPPMSVEHADIWVKSYSLYSRERYICNSGFKRKAGTSSLTE |
| | CVLNKATNVAHWTTPSLKCIREPKSSDKTHTCPPCPAPPVAGPS |
| | VFLFPPKPKDTLMISRTPEVTCVVVDVKHEDPEVKFNWYVDGV |
| | EVHNAKTKPREEQYNSTYRVVSVLTVLHQDWLNGKEYKCKVS |
| | NKALPAPIEKTISKAKGQPREPQVYTLPPSREQMTKNQVKLTCL |
| | VKGFYPSDIAVEWESNGQPENNYKTTPPVLDSDGSFFLYSKLTV |
| | DKSRWQQGNVFSCSVMHEALHNHYTQKSLSLSPGK |
| XENP23504 | Monomer 1 (IL-15 (Q108E)-first Fc domain). SEQ ID NO: 29 |
| | NWVNVISDLKKIEDLIQSMHIDATLYTESDVHPSCKVTAMKCF |
| | LLELQVISLESGDASIHDTVENLIILANNSLSSNGNVTESGCKEC |
| | EELEEKNIKEFLQSFVHIVEMFINTSGGGGSEPKSSDKTHTCPPC |
| | PAPPVAGPSVFLFPPKPKDTLMISRTPEVTCVVVDVKHEDPEVK |
| | FNWYVDGVEVHNAKTKPREEEYNSTYRVVSVLTVLHQDWLN |
| | GKEYKCKVSNKALPAPIEKTISKAKGQPREPQVYTLPPSREEMT |
| | KNQVSLTCDVSGFYPSDIAVEWESDGQPENNYKTTPPVLDSDG |
| | SFFLYSKLTVDKSRWEQGDVFSCSVLHEALHSHYTQKSLSLSPG |
| | K |
| | Monomer 2 (IL-15Rα-second Fc domain). SEQ ID NO: 30 |
| | ITCPPPMSVEHADIWVKSYSLYSRERYICNSGFKRKAGTSSLTE |
| | CVLNKATNVAHWTTPSLKCIRGGGGSEPKSSDKTHTCPPCPAPP |
| | VAGPSVFLFPPKPKDTLMISRTPEVTCVVVDVKHEDPEVKFNW |
| | YVDGVEVHNAKTKPREEQYNSTYRVVSVLTVLHQDWLNGKE |
| | YKCKVSNKALPAPIEKTISKAKGQPREPQVYTLPPSREQMTKNQ |
| | VKLTCLVKGFYPSDIAVEWESNGQPENNYKTTPPVLDSDGSFFL |
| | YSKLTVDKSRWQQGNVFSCSVLHEALHSHYTQKSLSLSPGK |
| XENP23343 | Monomer 1 (IL-15 (N65D)-first Fc domain). SEQ ID NO: 31 |
| | NWVNVISDLKKIEDLIQSMHIDATLYTESDVHPSCKVTAMKCF |
| | LLELQVISLESGDASIHDTVEDLIILANNSLSSNGNVTESGCKEC |
| | EELEEKNIKEFLQSFVHIVQMFINTSGGGGSEPKSSDKTHTCPPC |
| | PAPPVAGPSVFLFPPKPKDTLMISRTPEVTCVVVDVKHEDPEVK |
| | FNWYVDGVEVHNAKTKPREEEYNSTYRVVSVLTVLHQDWLN |
| | GKEYKCKVSNKALPAPIEKTISKAKGQPREPQVYTLPPSREEMT |
| | KNQVSLTCDVSGFYPSDIAVEWESDGQPENNYKTTPPVLDSDG |
| | SFFLYSKLTVDKSRWEQGDVFSCSVLHEALHSHYTQKSLSLSPG |
| | K |
| | Monomer 2 (IL-15Rα-second Fc domain). SEQ ID NO: 32 |
| | ITCPPPMSVEHADIWVKSYSLYSRERYICNSGFKRKAGTSSLTE |
| | CVLNKATNVAHWTTPSLKCIRGGGGSEPKSSDKTHTCPPCPAPP |
| | VAGPSVFLFPPKPKDTLMISRTPEVTCVVVDVKHEDPEVKFNW |
| | YVDGVEVHNAKTKPREEQYNSTYRVVSVLTVLHQDWLNGKE |
| | YKCKVSNKALPAPIEKTISKAKGQPREPQVYTLPPSREQMTKNQ |
| | VKLTCLVKGFYPSDIAVEWESNGQPENNYKTTPPVLDSDGSFFL |
| | YSKLTVDKSRWQQGNVFSCSVLHEALHSHYTQKSLSLSPGK |
| XENP24113 | Monomer 1 (IL-15 (N4D/N65D)-first Fc domain). SEQ ID NO: 33 |
| | NWVDVISDLKKIEDLIQSMHIDATLYTESDVHPSCKVTAMKCF |
| | LLELQVISLESGDASIHDTVEDLIILANNSLSSNGNVTESGCKEC |
| | EELEEKNIKEFLQSFVHIVQMFINTSGGGGSEPKSSDKTHTCPPC |
| | PAPPVAGPSVFLFPPKPKDTLMISRTPEVTCVVVDVKHEDPEVK |
| | FNWYVDGVEVHNAKTKPREEEYNSTYRVVSVLTVLHQDWLN |
| | GKEYKCKVSNKALPAPIEKTISKAKGQPREPQVYTLPPSREEMT |
| | KNQVSLTCDVSGFYPSDIAVEWESDGQPENNYKTTPPVLDSDG |
| | SFFLYSKLTVDKSRWEQGDVFSCSVLHEALHSHYTQKSLSLSPG |
| | K |
| | Monomer 2 (IL-15Rα-second Fc domain), SEQ ID NO: 34 |
| | ITCPPPMSVEHADIWVKSYSLYSRERYICNSGFKRKAGTSSLTE |
| | CVLNKATNVAHWTTPSLKCIRGGGGSEPKSSDKTHTCPPCPAPP |
| | VAGPSVFLFPPKPKDTLMISRTPEVTCVVVDVKHEDPEVKFNW |
| | YVDGVEVHNAKTKPREEQYNSTYRVVSVLTVLHQDWLNGKE |
| | YKCKVSNKALPAPIEKTISKAKGQPREPQVYTLPPSREQMTKNQ |
| | VKLTCLVKGFYPSDIAVEWESNGQPENNYKTTPPVLDSDGSFFL |
| | YSKLTVDKSRWQQGNVFSCSVLHEALHSHYTQKSLSLSPGK |
| XENP24341 | Monomer 1 (IL-15 (NID/N65D)-first Fc domain). SEQ ID NO: 35 |
| | DWVNVISDLKKIEDLIQSMHIDATLYTESDVHPSCKVTAMKCF |
| | LLELQVISLESGDASIHDTVEDLIILANNSLSSNGNVTESGCKEC |
| | EELEEKNIKEFLQSFVHIVQMFINTSEPKSSDKTHTCPPCPAPPV |
| | AGPSVFLFPPKPKDTLMISRTPEVTCVVVDVKHEDPEVKFNWY |
| | VDGVEVHNAKTKPREEEYNSTYRVVSVLTVLHQDWLNGKEY |
| | KCKVSNKALPAPIEKTISKAKGQPREPQVYTLPPSREEMTKNQV |
| | SLTCDVSGFYPSDIAVEWESDGQPENNYKTTPPVLDSDGSFFLY |
| | SKLTVDKSRWEQGDVFSCSVLHEALHSHYTQKSLSLSPGK |
| | Monomer 2 (IL-15Rα-second Fc domain). SEQ ID NO: 36 |
| | ITCPPPMSVEHADIWVKSYSLYSRERYICNSGFKRKAGTSSLTE |
| | CVLNKATNVAHWTTPSLKCIREPKSSDKTHTCPPCPAPPVAGPS |
| | VFLFPPKPKDTLMISRTPEVTCVVVDVKHEDPEVKFNWYVDGV |
| | EVHNAKTKPREEQYNSTYRVVSVLTVLHQDWLNGKEYKCKVS |
| | NKALPAPIEKTISKAKGQPREPQVYTLPPSREQMTKNQVKLTCL |
| | VKGFYPSDIAVEWESNGQPENNYKTTPPVLDSDGSFFLYSKLTV |
| | DKSRWQQGNVFSCSVLHEALHSHYTQKSLSLSPGK |
| XENP24301 | Monomer 1 (IL-15 (N4D/N65D)-first Fc domain). SEQ ID NO: 37 |
| | NWVDVISDLKKIEDLIQSMHIDATLYTESDVHPSCKVTAMKCF |
| | LLELQVISLESGDASIHDTVEDLIILANNSLSSNGNVTESGCKEC |
| | EELEEKNIKEFLQSFVHIVQMFINTSEPKSSDKTHTCPPCPAPPV |
| | AGPSVFLFPPKPKDTLMISRTPEVTCVVVDVKHEDPEVKFNWY |
| | VDGVEVHNAKTKPREEEYNSTYRVVSVLTVLHQDWLNGKEY |
| | KCKVSNKALPAPIEKTISKAKGQPREPQVYTLPPSREEMTKNQV |
| | SLTCDVSGFYPSDIAVEWESDGQPENNYKTTPPVLDSDGSFFLY |
| | SKLTVDKSRWEQGDVFSCSVLHEALHSHYTQKSLSLSPGK |
| | Monomer 2 (IL-15Rα-second Fc domain). SEQ ID NO: 38 |
| | ITCPPPMSVEHADIWVKSYSLYSRERYICNSGFKRKAGTSSLTE |
| | CVLNKATNVAHWTTPSLKCIREPKSSDKTHTCPPCPAPPVAGPS |
| | VFLFPPKPKDTLMISRTPEVTCVVVDVKHEDPEVKFNWYVDGV |
| | EVHNAKTKPREEQYNSTYRVVSVLTVLHQDWLNGKEYKCKVS |
| | NKALPAPIEKTISKAKGQPREPQVYTLPPSREQMTKNQVKLTCL |
| | VKGFYPSDIAVEWESNGQPENNYKTTPPVLDSDGSFFLYSKLTV |
| | DKSRWQQGNVFSCSVLHEALHSHYTQKSLSLSPGK |

Methods of Treatment with IL15-IL15Rα Heterodimeric Fc-Fusion Proteins

In one aspect, the present disclosure provides methods of treating a blood cancer in a subject in need thereof, the method comprising administering to the subject a therapeutically effective amount of any of the heterodimeric proteins disclosed herein or any combinations thereof.

In another aspect, the present disclosure provides any of the heterodimeric protein disclosed herein or any combinations thereof, for use in the treatment of a blood cancer in a subject in need thereof.

In another aspect, the present disclosure provides the use of a therapeutically effective amount of any of the heterodimeric proteins as disclosed herein or any combinations thereof, in the manufacture of a medicament for the treatment of a blood cancer in a subject in need thereof.

A blood cancer refers to an abnormal or excessive production of blood cells (e.g., white blood cells). Examples of blood cancers to be treated by the methods and uses disclosed herein include, but are not limited, leukemias, lymphomas, and myelomas. More particular non-limiting examples of such blood cancers include acute myeloid leukemia, adult acute lymphoblastic leukemia, chronic lymphocytic leukemia, non-Hodgkin's lymphoma, B-cell non-Hodgkin's lymphoma, and multiple myeloma. In some embodiments, the blood cancer is a relapsed or refractory. In some embodiments, the blood cancer is selected from the group consisting of leukemia, acute myeloid leukemia, adult acute lymphoblastic leukemia, chronic lymphocytic leukemia, lymphoma, non-Hodgkin's lymphoma, B-cell non-Hodgkin's lymphoma, and multiple myeloma. In some embodiments, the blood cancer is selected from the group consisting of leukemia, acute myeloid leukemia, adult acute lymphoblastic leukemia, chronic lymphocytic leukemia. In some embodiments, the blood cancer is selected from the group consisting of lymphoma, non-Hodgkin's lymphoma, B-cell non-Hodgkin's lymphoma. In some embodiments, the blood cancer is leukemia. In some embodiments, the blood cancer is acute myeloid leukemia. In some embodiments, the blood cancer is adult acute lymphoblastic leukemia. In some embodiments, the blood cancer is chronic lymphocytic leukemia. In some embodiments, the blood cancer is lymphoma. In some embodiments, the blood cancer is non-Hodgkin's lymphoma. In some embodiments, the blood cancer is B-cell non-Hodgkin's lymphoma. In some embodiments, the blood cancer is multiple myeloma. In some embodiments, the blood cancer is relapsed or refractory multiple myeloma. In some embodiments, the blood cancer is a blood cancer for which standard therapy does not exist, has proven to be ineffective or intolerable, or is considered inappropriate, or for whom a clinical trial of an investigational agent is a recognized standard of care.

In some embodiments, a combination of two or more (e.g., 2, 3, 4, 5, 6, etc.) heterodimeric proteins are used in the methods described herein. In some embodiments, a combination of a first heterodimeric protein and a second heterodimeric protein is administered to the subject.

In some embodiments, the first heterodimeric protein comprises a first monomer comprising the amino acid sequence set forth in SEQ ID NO: 9, and a second monomer comprising the amino acid sequence set forth in SEQ ID NO: 10; and a second heterodimeric protein comprises a first monomer comprising the amino acid sequence set forth in SEQ ID NO: 9, and a second monomer comprising the amino acid sequence set forth in SEQ ID NO: 16.

In some embodiments, the first heterodimeric protein represents about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 75%, about 70%, about 65%, about 60%, about 55%, about 50%, about 45%, about 40%, about 35%, about 30%, about 25%, about 20%, about 15%, about 10%, or about 5% of the heterodimeric protein in the combination. In some embodiments, the first heterodimeric protein represents about 85% of the heterodimeric protein in the combination. In some embodiments, the first heterodimeric protein represents about 84% of the heterodimeric protein in the combination. In some embodiments, the first heterodimeric protein represents about 83% of the heterodimeric protein in the combination. In some embodiments, the first heterodimeric protein represents about 82% of the heterodimeric protein in the combination. In some embodiments, the first heterodimeric protein represents about 81% of the heterodimeric protein in the combination. In some embodiments, the first heterodimeric protein represents about 80% of the heterodimeric protein in the combination.

In some embodiments, the second heterodimeric protein represents about 95%, about 90%, about 85%, about 80%, about 75%, about 70%, about 75%, about 70%, about 65%, about 55%, about 50%, about 45%, about 40%, about 35%, about 30%, about 25%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2% or about 1% of the combination. In some embodiments, the second heterodimeric protein represents about 15% of the heterodimeric protein in the combination. In some embodiments, the second heterodimeric protein represents about 16% of the heterodimeric protein in the combination. In some embodiments, the second heterodimeric protein represents about 17% of the heterodimeric protein in the combination. In some embodiments, the second heterodimeric protein represents about 18% of the heterodimeric protein in the combination. In some embodiments, the second heterodimeric protein represents about 19% of the heterodimeric protein in the combination. In some embodiments, the second heterodimeric protein represents about 20% of the heterodimeric protein in the combination.

In some embodiments, the first heterodimeric protein represents between about 50-about 100%, about 70-about 95%, about 80-about 90%, or about 80-about 85% of the heterodimeric protein in the combination. In some embodiments of any of the methods disclosed herein, the second heterodimeric protein represents between about 1-about 50%, about 5-about 30%, about 10-about 20%, or about 15-about 20% of the heterodimeric protein in the combination. In some embodiments, the first heterodimeric protein represents about 85% of the heterodimeric protein in the combination, and the second heterodimeric protein represents about 15% of the heterodimeric protein in the combination. In some embodiments, the first heterodimeric protein represents about 84% of the heterodimeric protein in the combination, and the second heterodimeric protein represents about 16% of the heterodimeric protein in the combination. In some embodiments, the first heterodimeric protein represents about 83% of the heterodimeric protein in the combination, and the second heterodimeric protein represents about 17% of the heterodimeric protein in the combination. In some embodiments, the first heterodimeric protein represents about 82% of the heterodimeric protein in the combination, and the second heterodimeric protein represents about 18% of the heterodimeric protein in the combination. In some embodiments, the first heterodimeric protein represents about 81% of the heterodimeric protein in the combination, and the second heterodimeric protein represents about 19% of the heterodimeric protein in the combination. In some embodiments, the first heterodimeric protein represents about 80% of the heterodimeric protein in the combination, and the second heterodimeric protein represents about 20% of the heterodimeric protein in the combination.

In some embodiments, the first heterodimeric protein represents 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, 60%, 55%, 50%, 45%, 40%, 35%, 30%, 25%, 20%, 15%, 10%, or 5% of the heterodimeric protein in the combination. In some embodiments, the first heterodimeric protein represents 85% of the heterodimeric protein in the combination. In some embodiments, the first heterodimeric protein represents 84% of the heterodimeric protein in the combination. In some embodiments, the first heterodimeric protein represents 83% of the heterodimeric protein in the combination. In some embodiments, the first heterodimeric protein represents 82% of the heterodimeric protein in the combination. In some embodiments, the first heterodimeric protein represents 81% of the heterodimeric protein in the combination. In some embodiments, the first heterodimeric protein represents 80% of the heterodimeric protein in the combination.

In some embodiments, the second heterodimeric protein represents 95%, 90%, 85%, 80%, 75%, 70%, 75%, 70%, 65%, 55%, 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2% or 1% of the combination. In some embodiments, the second heterodimeric protein represents 15% of the heterodimeric protein in the combination. In some embodiments, the second heterodimeric protein represents 16% of the heterodimeric protein in the combination. In some embodiments, the second heterodimeric protein represents 17% of the heterodimeric protein in the combination. In some embodiments, the second heterodimeric protein represents 18% of the heterodimeric protein in the combination. In some embodiments, the second heterodimeric protein represents 19% of the heterodimeric protein in the combination. In some embodiments, the second heterodimeric protein represents 20% of the heterodimeric protein in the combination.

In some embodiments, the first heterodimeric protein represents between 50-100%, 70-95%, 80-90%, or 80-85% of the heterodimeric protein in the combination. In some embodiments of any of the methods disclosed herein, the second heterodimeric protein represents between 1-50%, 5-30%, 10-20%, or 15-20% of the heterodimeric protein in the combination. In some embodiments, the first heterodimeric protein represents 85% of the heterodimeric protein in the combination, and the second heterodimeric protein represents 15% of the heterodimeric protein in the combination. In some embodiments, the first heterodimeric protein represents 84% of the heterodimeric protein in the combination, and the second heterodimeric protein represents 16% of the heterodimeric protein in the combination. In some embodiments, the first heterodimeric protein represents 83% of the heterodimeric protein in the combination, and the second heterodimeric protein represents 17% of the heterodimeric protein in the combination. In some embodiments, the first heterodimeric protein represents 82% of the heterodimeric protein in the combination, and the second heterodimeric protein represents 18% of the heterodimeric protein in the combination. In some embodiments, the first heterodimeric protein represents 81% of the heterodimeric protein in the combination, and the second heterodimeric protein represents 19% of the heterodimeric protein in the combination. In some embodiments, the first heterodimeric protein represents 80% of the heterodimeric protein in the combination, and the second heterodimeric protein represents 20% of the heterodimeric protein in the combination.

In some embodiments, said first and second heterodimeric proteins are administered simultaneously. In some embodiments, said first and second heterodimeric proteins are administered sequentially. In some embodiments, the first heterodimeric protein is administered before the second heterodimeric protein. In some embodiments, the second heterodimeric protein is administered before the first heterodimeric protein. In some embodiments, said first and second heterodimeric proteins are administered in the same composition. In some embodiments, the first and second heterodimeric proteins are administered in separate compositions.

The methods and uses herein described include administering to the subject a therapeutically effective amount of any of the heterodimeric proteins described herein, or a combination thereof, or a composition described herein to produce such effect. Identifying a subject in need of such treatment can be in the judgment of a subject or a health care professional and can be subjective (e.g. opinion) or objective (e.g. measurable by a test or diagnostic method). Such treatment will be suitably administered to subjects suffering from, having, susceptible to, or at risk for the blood cancer.

In another aspect, the present disclosure provides methods for inducing the proliferation of CD8⁻ effector memory T cells in a subject suffering from a blood cancer, the method comprising administering to the subject an effective amount of any of the heterodimeric proteins disclosed herein or any combinations thereof.

In another aspect, the present disclosure provides methods for inducing the proliferation of NK cells in a subject suffering from a blood cancer, the method comprising administering to the subject an effective amount of any of the heterodimeric proteins disclosed herein or any combinations thereof.

In another aspect, the present disclosure provides methods for inducing the proliferation of NK cells in a subject suffering from a blood cancer, the method comprising administering to the subject an effective amount of any of the heterodimeric proteins disclosed herein or any combinations thereof, and wherein the proliferative response of NK cells is stronger than the proliferative response of CD8⁺ effector memory T cells upon the administration of an effective amount of any of the heterodimeric proteins disclosed herein or any combinations thereof.

In another aspect, the present disclosure provides methods for inducing the proliferation of CD8⁻ effector memory T cells and NK cells in a subject suffering from a blood cancer, the method comprising administering to the subject an effective amount of any of the heterodimeric proteins disclosed herein or any combinations thereof. In some embodiments, the proliferative response of NK cells is stronger than the proliferative response of CD8⁺ effector memory T cells upon the administration of an effective amount of any of the heterodimeric proteins disclosed herein or any combinations thereof.

In another aspect, the present disclosure provides methods for inducing the proliferation of CD4⁻ effector memory T cells in a subject suffering from a blood cancer, the method comprising administering to the subject an effective amount of any of the heterodimeric proteins disclosed herein or any combinations thereof.

In another aspect, the present disclosure provides methods for inducing IFNγ production in a subject suffering from a blood cancer, the method comprising administering to the subject an effective amount of any of the heterodimeric proteins disclosed herein or any combinations thereof.

The heterodimeric protein (or combination thereof) may be administered parenterally. In some embodiments, the parenteral administration is intravenous.

In some embodiments, the heterodimeric protein of the disclosure is administered systemically. In some embodiments, the heterodimeric protein is administered as a composition comprising a pharmaceutically acceptable buffer. Suitable carriers and their formulations are described, for example, in Remington's Pharmaceutical Sciences by E. W. Martin. In some embodiments, the heterodimeric protein is provided in a dosage form that is suitable for parenteral (e.g., intravenous) administration route.

Compositions comprising the heterodimeric protein may be provided in unit dosage forms (e.g., in single-dose ampoules, syringes or bags). In some embodiments, the heterodimeric protein is provided in vials containing several doses. A suitable preservative may be added to the composition (see below). The composition may be in the form of a solution, a suspension, an emulsion, an infusion device, or a delivery device for implantation, or it may be presented as a dry powder to be reconstituted with water or another suitable vehicle before use. Apart from the heterodimeric proteins disclosed herein, the composition may include suitable acceptable carriers and/or excipients. In some embodiments, the composition is suitable for parenteral administration. The heterodimeric protein(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

The pharmaceutical compositions comprising the heterodimeric protein may be in a form suitable for sterile injection. To prepare such a composition, the protein is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate).

The amount of the heterodimeric protein of the disclosure to be administered varies depending upon the manner of administration, the age and body weight of the patient, and the clinical symptoms of the cancer to be treated. Human dosage amounts can initially be determined by extrapolating from the amount of protein used in mice or non-human primates. In certain embodiments, the dosage may vary from between about 0.0001 mg protein/kg body weight to about 5 mg protein/kg body weight; or from about 0.001 mg/kg body weight to about 4 mg/kg body weight or from about 0.005 mg/kg body weight to about 1 mg/kg body weight or from about 0.005 mg/kg body weight to about 0.3 mg/kg body weight or from about 0.005 mg/kg body weight to about 0.2 mg/kg body weight or from about 0.005 mg/kg body weight to about 0.02 mg/kg body weight. In some embodiments, this dose may be about 0.0001, about 0.00025, about 0.0003, about 0.0005, about 0.001, about 0.003, about 0.005, about 0.008, about 0.01, about 0.015, about 0.02, about 0.03, about 0.04, about 0.05, about 0.06, about 0.07, about 0.08, about 0.09, about 0.1, about 0.12, about 0.135, about 0.15, about 0.16, about 0.2, about 0.2025, about 0.24, about 0.25, about 0.3, about 0.32, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.15, about 1.2, about 1.25, about 1.3, about 1.35, about 1.4, about 1.45, about 1.5, about 1.6, about 1.7, about 1.8, about 1.9, about 2, about 2.5, about 3, about 3.5, about 4, about 4.5, or about 5 mg/kg body weight. In some embodiments, the dose is about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.1 mg/kg, about 0.12 mg/kg, about 0.16 mg/kg, about 0.2 mg/kg, about 0.24 mg/kg and about 0.32 mg/kg body weight. In some embodiments, the dosage is about 0.0025 mg/kg body weight. In some embodiments, the dosage is about 0.01 mg/kg body weight. In some embodiments, the dosage is about 0.015 mg/kg body weight. In some embodiments, the dosage is about 0.02 mg/kg body weight. In some embodiments, the dosage is about 0.03 mg/kg body weight. In some embodiments, the dosage is about 0.04 mg/kg body weight. In some embodiments, the dosage is about 0.06 mg/kg body weight. In some embodiments, the dosage is about 0.08 mg/kg body weight. In some embodiments, the dosage is about 0.09 mg/kg body weight. In some embodiments, the dosage is about 0.12 mg/kg body weight. In some embodiments, the dosage is about 0.135 mg/kg body weight. In some embodiments, the dosage is about 0.16 mg/kg body weight. In some embodiments, the dosage is about 0.2025 mg/kg body weight. In some embodiments, the dosage is about 0.24 mg/kg body weight. In some embodiments, the dosage is about 0.32 mg/kg body weight. In some embodiments, the heterodimeric protein of the disclosure is administered by IV infusion according to these dosages.

In certain embodiments, the dosage may vary from between 0.0001 mg protein/kg body weight to 5 mg protein/kg body weight; or from 0.001 mg/kg body weight to 4 mg/kg body weight or from 0.005 mg/kg body weight to 1 mg/kg body weight or from 0.005 mg/kg body weight to 0.3 mg/kg body weight or from 0.005 mg/kg body weight to 0.2 mg/kg body weight or from 0.005 mg/kg body weight to 0.02 mg/kg body weight. In some embodiments, this dose may be 0.0001, 0.0003, 0.0005, 0.001, 0.003, 0.005, 0.008, 0.01, 0.015, 0.02, 0.03, 0.05, 0.08, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.15, 1.2, 1.25, 1.3, 1.35, 1.4, 1.45, 1.5, 1.6, 1.7, 1.8, 1.9, 2, 2.5, 3, 3.5, 4, 4.5, or 5 mg/kg body weight. In some embodiments, the dose is selected from the group consisting of 0.0025 mg/kg, 0.005 mg/kg, 0.01 mg/kg, 0.015 mg/kg, 0.02 mg/kg, 0.025 mg/kg, 0.03 mg/kg, 0.04 mg/kg, 0.05 mg/kg, 0.06 mg/kg, 0.08 mg/kg, 0.09 mg/kg, 0.10 mg/kg, 0.12 mg/kg, 0.135 mg/kg, 0.16 mg/kg, 0.20 mg/kg, 0.2025 mg/kg, 0.24 mg/kg and 0.32 mg/kg body weight. In some embodiments, the dosage is 0.0025 mg/kg body weight. In some embodiments, the dosage is 0.01 mg/kg body weight. In some embodiments, the dosage is 0.015 mg/kg body weight. In some embodiments, the dosage is 0.02 mg/kg body weight. In some embodiments, the dosage is 0.03 mg/kg body weight. In some embodiments, the dosage is 0.04 mg/kg body weight. In some embodiments, the dosage is 0.06 mg/kg body weight. In some embodiments, the dosage is 0.08 mg/kg body weight. In some embodiments, the dosage is 0.09 mg/kg body weight. In some embodiments, the dosage is 0.12 mg/kg body weight. In some embodiments, the dosage is 0.135 mg/kg body weight. In some embodiments, the dosage is 0.16 mg/kg body weight. In some embodiments, the dosage is 0.2025 mg/kg body weight. In some embodiments, the dosage is 0.24 mg/kg body weight. In some embodiments, the dosage is 0.32 mg/kg body weight. In some embodiments, the heterodimeric protein of the disclosure is administered by IV infusion according to these dosages.

In certain embodiments, the dosage of the combination of heterodimeric proteins may vary from between about 0.0001 mg protein/kg body weight to about 5 mg protein/kg body weight; or from about 0.001 mg/kg body weight to about 4 mg/kg body weight or from about 0.005 mg/kg body weight to about 1 mg/kg body weight or from about 0.005 mg/kg body weight to about 0.3 mg/kg body weight or from about 0.005 mg/kg body weight to about 0.2 mg/kg body weight or from about 0.005 mg/kg body weight to about 0.02 mg/kg body weight. In some embodiments, this dose may be about 0.0001, about 0.0003, about 0.0005, about 0.001, about 0.003, about 0.005, about 0.008, about 0.01, about 0.015, about 0.02, about 0.03, about 0.05, about 0.08, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.15, about 1.2, about 1.25, about 1.3, about 1.35, about 1.4, about 1.45, about 1.5, about 1.6, about 1.7, about 1.8, about 1.9, about 2, about 2.5, about 3, about 3.5, about 4, about 4.5, or about 5 mg/kg body weight. In some embodiments, the dose is about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.10 mg/kg, about 0.12 mg/kg, about 0.16 mg/kg, about 0.20 mg/kg, about 0.24 mg/kg and about 0.32 mg/kg body weight. In some embodiments, the dosage is about 0.0025 mg/kg body weight. In some embodiments, the dosage is about 0.01 mg/kg body weight. In some embodiments, the dosage is about 0.015 mg/kg body weight. In some embodiments, the dosage is about 0.02 mg/kg body weight. In some embodiments, the dosage is about 0.03 mg/kg body weight. In some embodiments, the dosage is about 0.04 mg/kg body weight. In some embodiments, the dosage is about 0.06 mg/kg body weight. In some embodiments, the dosage is about 0.08 mg/kg body weight. In some embodiments, the dosage is about 0.09 mg/kg body weight. In some embodiments, the dosage is about 0.12 mg/kg body weight. In some embodiments, the dosage is about 0.135 mg/kg body weight. In some embodiments, the dosage is about 0.16 mg/kg body weight. In some embodiments, the dosage is about 0.2025 mg/kg body weight. In some embodiments, the dosage is about 0.24 mg/kg body weight. In some embodiments, the dosage is about 0.32 mg/kg body weight. In some embodiments, the combination of heterodimeric proteins of the disclosure is administered by IV infusion according to these dosages.

In certain embodiments, the dosage of the combination of heterodimeric proteins may vary from between 0.0001 mg protein/kg body weight to 5 mg protein/kg body weight; or from 0.001 mg/kg body weight to 4 mg/kg body weight or from 0.005 mg/kg body weight to 1 mg/kg body weight or from 0.005 mg/kg body weight to 0.3 mg/kg body weight or from 0.005 mg/kg body weight to 0.2 mg/kg body weight or from 0.005 mg/kg body weight to 0.02 mg/kg body weight. In some embodiments, this dose may be 0.0001, 0.0003, 0.0005, 0.001, 0.003, 0.005, 0.008, 0.01, 0.015, 0.02, 0.03, 0.05, 0.08, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.15, 1.2, 1.25, 1.3, 1.35, 1.4, 1.45, 1.5, 1.6, 1.7, 1.8, 1.9, 2, 2.5, 3, 3.5, 4, 4.5, or 5 mg/kg body weight. In some embodiments, the dose is 0.0025 mg/kg, 0.005 mg/kg, 0.01 mg/kg, 0.015 mg/kg, 0.02 mg/kg, 0.025 mg/kg, 0.03 mg/kg, 0.04 mg/kg, 0.05 mg/kg, 0.06 mg/kg, 0.08 mg/kg, 0.10 mg/kg, 0.12 mg/kg, 0.16 mg/kg, 0.20 mg/kg, 0.24 mg/kg and 0.32 mg/kg body weight. In some embodiments, the dosage is 0.0025 mg/kg body weight. In some embodiments, the dosage is 0.01 mg/kg body weight. In some embodiments, the dosage is 0.015 mg/kg body weight. In some embodiments, the dosage is 0.02 mg/kg body weight. In some embodiments, the dosage is 0.03 mg/kg body weight. In some embodiments, the dosage is 0.04 mg/kg body weight. In some embodiments, the dosage is 0.06 mg/kg body weight. In some embodiments, the dosage is 0.08 mg/kg body weight. In some embodiments, the dosage is 0.09 mg/kg body weight. In some embodiments, the dosage is 0.12 mg/kg body weight. In some embodiments, the dosage is 0.135 mg/kg body weight. In some embodiments, the dosage is 0.16 mg/kg body weight. In some embodiments, the dosage is 0.2025 mg/kg body weight. In some embodiments, the dosage is 0.24 mg/kg body weight. In some embodiments, the dosage is 0.32 mg/kg body weight. In some embodiments, the combination of heterodimeric proteins of the disclosure is administered by IV infusion according to these dosages.

In some embodiments, the heterodimeric protein of the disclosure, or a combination thereof, is administered daily, i.e., every 24 hours. In some embodiments, the heterodimeric protein or a combination thereof is administered weekly, i.e., once per week (Q1W). In some embodiments, the heterodimeric protein or a combination thereof is administered once every two weeks, i.e., once every 14 days (Q2W). In some embodiments, the heterodimeric protein or a combination thereof is administered once every three weeks, i.e., once every 21 days (Q3W). In some embodiments, the heterodimeric protein or a combination thereof is administered once every four weeks, i.e., once every 28 days (Q4W). In some embodiments, the heterodimeric protein or a combination thereof is administered once every five weeks (Q5W). In some embodiments, the heterodimeric protein or a combination thereof is administered once every six weeks (Q6W). In some embodiments, the heterodimeric protein or a combination thereof is administered once every seven weeks (Q7W). In some embodiments, the heterodimeric protein or a combination thereof is administered once every eight weeks (Q8W). In some embodiments, the heterodimeric protein or a combination thereof is administered once every nine weeks (Q9W). In some embodiments, the heterodimeric protein or a combination thereof is administered once every ten weeks (Q10W). In some embodiments, the heterodimeric protein or a combination thereof is administered once every eleven weeks (Q11W). In some embodiments, the heterodimeric protein or a combination thereof is administered once every twelve weeks (Q12W). In some embodiments, the heterodimeric protein or a combination thereof is administered once every month. In some embodiments, the heterodimeric protein or a combination thereof is administered once every two months. In some embodiments, the heterodimeric protein or a combination thereof is administered once every three months. In some embodiments, the heterodimeric protein or a combination thereof is administered once every four months. In some embodiments, the heterodimeric protein or a combination thereof is administered once every five months. In some embodiments, the heterodimeric protein or a combination thereof is administered once every six months. In some embodiments, the heterodimeric protein or a combination thereof is administered once every seven months. In some embodiments, the heterodimeric protein or a combination thereof is administered once every eight months. In some embodiments, the heterodimeric protein or a combination thereof is administered once every nine months. In some embodiments, the heterodimeric protein or a combination thereof is administered once every ten months. In some embodiments, the heterodimeric protein or a combination thereof is administered once every eleven months. In some embodiments, the heterodimeric protein or a combination thereof is administered once every twelve months. In some embodiments, the heterodimeric protein or a combination thereof is administered once every year. In some embodiments, the heterodimeric protein or a combination thereof of the disclosure is administered by IV infusion according to the frequency disclosed herein.

In some embodiments, the heterodimeric protein or a combination thereof of the disclosure is administered at any of the above frequencies in one or more cycles. In some embodiments, the heterodimeric protein or a combination thereof of the disclosure is administered at any of the above frequencies in 1, 2, 3, 4, 5, 6, 7, 8, 9, 10, 11, 12, 13, 14, 15, 16, 17, 18, 19 or 20 cycles. In some embodiments, the heterodimeric protein or a combination thereof of the disclosure is administered at a frequency of Q1W in one or more cycles. In some embodiments, the heterodimeric protein or a combination thereof of the disclosure is administered at a frequency of Q1W in 1, 2, 3, 4, 5, 6, 7, 8, 9, 10, 11, 12, 13, 14, 15, 16, 17, 18, 19 or 20 cycles. In some embodiments, the heterodimeric protein or a combination thereof of the disclosure is administered at a frequency of Q2W in one or more cycles. In some embodiments, the heterodimeric protein or a combination thereof of the disclosure is administered at a frequency of Q2W in 1, 2, 3, 4, 5, 6, 7, 8, 9, 10, 11, 12, 13, 14, 15, 16, 17, 18, 19 or 20 cycles. In some embodiments, the heterodimeric protein or a combination thereof of the disclosure is administered at a frequency of Q3W in one or more cycles. In some embodiments, the heterodimeric protein or a combination thereof of the disclosure is administered at a frequency of Q3W in 1, 2, 3, 4, 5, 6, 7, 8, 9, 10, 11, 12, 13, 14, 15, 16, 17, 18, 19 or 20 cycles. In some embodiments, the heterodimeric protein or a combination thereof of the disclosure is administered at a frequency of Q4W in one or more cycles. In some embodiments, the heterodimeric protein or a combination thereof of the disclosure is administered at a frequency of Q4W in 1, 2, 3, 4, 5, 6, 7, 8, 9, 10, 11, 12, 13, 14, 15, 16, 17, 18, 19 or 20 cycles.

In some embodiments, the subject has been previously administered one or more prior treatments or agents for treatment of the blood cancer. In some embodiments, the subject has been previously administered one prior treatment. In some embodiments, the subject has been previously administered two prior treatments. In some embodiments, the subject has been previously administered three prior treatments. In some embodiments, the subject has been previously administered four prior treatments. In some embodiments, the subject has been previously administered five prior treatments. In some embodiments, the prior treatment administered to the subject is an immunomodulatory drug, a proteasome inhibitor, an anti-CD38 monoclonal antibody, or a combination thereof. In some embodiments, the prior treatment administered to the subject is an immunomodulatory drug. In some embodiments, the immunomodulatory drug is selected from the group consisting of lenalidomide, thalidomide, and pomalidomide. In some embodiments, the immunomodulatory drug is lenalidomide. In some embodiments, the immunomodulatory drug is thalidomide. In some embodiments, the immunomodulatory drug is pomalidomide. In some embodiments, the prior treatment administered to the subject is a proteasome inhibitor. In some embodiments, the proteasome inhibitor is selected from the group consisting of bortezomib, carfilzomib, and ixazomib. In some embodiments, the proteasome inhibitor is selected from the group consisting of bortezomib. In some embodiments, the proteasome inhibitor is selected from the group consisting of carfilzomib. In some embodiments, the proteasome inhibitor is selected from the group consisting of ixazomib. In some embodiments, the prior treatment administered to the subject is an anti-CD38 monoclonal antibody. In some embodiments, the anti-CD38 antibody is selected from the group consisting of daratumumab, isatuximab, mezagitamab (TAK-079) and felzartamab (MOR202). In some embodiments, the anti-CD38 monoclonal antibody is daratumumab. In some embodiments, the anti-CD38 monoclonal antibody is isatuximab. In some embodiments, the anti-CD38 antibody is mezagitamab. In some embodiments, the anti-CD38 antibody is felzartamab.

Method of Treatment with IL15-IL15Rα Heterodimeric Fc-Fusion Proteins and an Anti-CD38 Antibody as Combination Therapy

Another aspect of the present disclosure provides a method of treating a blood cancer as disclosed herein in a subject in need thereof, the method comprising administering to the subject an effective amount of (a) any heterodimeric protein (i.e., IL15-IL15Rα heterodimeric Fc-fusion protein) disclosed herein or combinations thereof and (b) an anti-CD38 antibody or an antigen-binding fragment thereof. In some embodiments, the anti-CD38 antibody is a monoclonal antibody. The heterodimeric protein may be administered according to any of the herein disclosed methods. The heterodimeric protein may be administered in any of the herein disclosed compositions.

In some embodiments, two or more of the heterodimeric proteins as disclosed herein are administered to the subject. In some embodiments, three or more of the heterodimeric proteins as disclosed herein are administered to the subject. In some embodiments, four or more of the heterodimeric proteins as disclosed herein are administered to the subject. In some embodiments, five or more of the heterodimeric proteins as disclosed herein are administered to the subject.

In some embodiments, a combination of a first heterodimeric protein and a second heterodimeric protein is administered to the subject. In some embodiments, the first heterodimeric protein comprises a first monomer comprising the amino acid sequence set forth in SEQ ID NO: 9, and a second monomer comprising the amino acid sequence set forth in SEQ ID NO: 10; and a second heterodimeric protein comprises a first monomer comprising the amino acid sequence set forth in SEQ ID NO: 9, and a second monomer comprising the amino acid sequence set forth in SEQ ID NO: 16.

Cluster of differentiation 38 (CD38) is a type II transmembrane glycoprotein that is expressed on various hematopoietic and non-hematopoietic tissues and cells. The level of expression of CD38 on hematopoietic cells varies on the stage of maturation and activation of the cells. Tumor cells (e.g., leukemia cells and multiple myeloma cells) express CD38 at higher levels as compared with normal lymphoid and myeloid cells. Overexpression of CD38 has been associated with poor prognosis in patients with some blood cancers. Interruption of the CD38 pathways is an attractive strategy for reinvigorating tumor-specific T cell immunity, and indeed, multiple inhibitors of CD38 have demonstrated clinical efficacy or promising antitumor activity in a wide range of tumor types, including multiple myeloma and chronic lymphocytic leukemia and resulted in the approval of some anti-CD38 antibodies (e.g., daratumumab, isatuximab, mezagitamab (TAK-079) and felzartamab (MOR202)) for the treatment of select indications to date.

Antibodies that specifically bind to CD38 are known in the art and have been described, for example, de Weers et al. J Immunol. 2011; 186(3):1840-1848, Martin et al. Blood; 126(23):509, Fedyk et al. British J. of Clin. Pharm. 2020; 86(7):1314-1325, Boxhammer et al. Blood. 2015; 126(23):3015; WO2006/099875, WO2011/154453A1, WO2008/047242, WO2007/042309, WO2012/092612, WO2012/092616, WO2019/186273, U.S. Pat. No. 7,829,673B2, U.S. Pat. No. 9,187,565B2, U.S. Pat. No. 9,249,226B2, U.S. Pat. No. 9,944,711B2, U.S. Pat. No. 8,153,765B2, U.S. Pat. No. 8,263,746B2, U.S. Pat. No. 9,758,590B2, U.S. Pat. No. 8,088,896B2, U.S. Pat. No. 8,486,894B2, U.S. Pat. No. 9,193,799B2, U.S. Pat. No. 10,184,005B2, U.S. Pat. No. 9,102,744B2, U.S. Pat. No. 8,362,211B2, U.S. Pat. No. 8,926,969B2, U.S. Pat. No. 9,790,285B2, U.S. Pat. No. 9,676,869B2, U.S. Pat. No. 10,336,833B2, U.S. Pat. No. 10,494,444B2, US 2009/0148449A1, US 2011/0099647A1, US 2020/0283542A1, US 2013/0209355A1, US 2016/0237161A1, US 2009/0304710A1, US 2020/0408765A1, US 2010/0285004A1, US 2011/0268726A9, US 2016/0096901A1, US 2009/0252733A1, US 2012/0052078A1, US 2013/273072A1, US 2016/0075796, US 2019/0077877A1, US 2014/0155584A1, US 2012/0201827A1, US 2013/0171154A1, US 2015/0203587A1, US 2015/0291702A1, US 2018/0016349A1, US 2018/0066069A1, US 2020/0031951A1, US 2020/0040105A1, EP1866338, EP2567976, EP3153525, EP2580243, EP3613774, EP3498735, EP2860192, EP3284755, EP3284754, EP3798231, EP2658870, EP3789404, and EP2648871. Examples of anti-CD38 antibodies useful for the methods of the disclosure include, but are not limited to daratumumab, isatuximab, mezagitamab (TAK-079) and felzartamab (MOR202). Daratumumab is an IgGk1 monoclonal anti-CD38 antibody described in WO2006/099875 and de Weers et al. J Immunol. 2011; 186(3):1840-1848. Isatuximab is a monoclonal anti-CD38 antibody described in WO2008/047242 and Martin et al. Blood; 126(23):509. Mezagitamab is an antibody targeting CD38 described in WO2012/092612, WO2012/092616, and/or WO2019/186273 and Fedyk et al. British J. of Clin. Pharm. 2020; 86(7):1314-1325. Felzartamab is an antibody targeting CD38 described in U.S. Pat. No. 8,263,746B2 or WO2007/042309 and Boxhammer et al. Blood. 2015; 126(23):3015. In some embodiments, the anti-CD38 antibody is daratumumab. In some embodiments, the anti-CD-38 antibody is isatuximab. In some embodiments, the anti-CD-38 antibody is mezagitamab (TAK-079). In some embodiments, the anti-CD-38 antibody is felzartamab (MOR202).

In some embodiments, the anti-CD38 antibody or antigen-binding fragment thereof is administered in combination with XENP24306. In some embodiments, the anti-CD38 antibody or antigen-binding fragment thereof is administered in combination with XENP32803. In some embodiments, the anti-CD38 antibody or antigen-binding fragment thereof is administered in combination with XENP24306 and XENP32803. In some embodiments, daratumumab is administered in combination with XENP24306. In some embodiments, daratumumab is administered in combination with XENP32803. In some embodiments, daratumumab is administered in combination with XENP24306 and XENP32803. In some embodiments, daratumumab or an antigen-binding fragment thereof is administered in combination with XENP24306. In some embodiments, daratumumab or an antigen-binding fragment thereof is administered in combination with XENP32803. In some embodiments, daratumumab or an antigen-binding fragment thereof is administered in combination with XENP24306 and XENP32803. In some embodiments, isatuximab is administered in combination with XENP24306. In some embodiments, isatuximab is administered in combination with XENP32803. In some embodiments, isatuximab is administered in combination with XENP24306 and XENP32803. In some embodiments, isatuximab or an antigen-binding fragment thereof is administered in combination with XENP24306. In some embodiments, isatuximab or an antigen-binding fragment thereof is administered in combination with XENP32803. In some embodiments, isatuximab or an antigen-binding fragment thereof is administered in combination with XENP24306 and XENP32803. In some embodiments, mezagitamab (TAK-079) is administered in combination with XENP24306. In some embodiments, mezagitamab (TAK-079) is administered in combination with XENP32803. In some embodiments, mezagitamab (TAK-079) is administered in combination with XENP24306 and XENP32803. In some embodiments, mezagitamab (TAK-079) or an antigen-binding fragment thereof is administered in combination with XENP24306. In some embodiments, mezagitamab (TAK-079) or an antigen-binding fragment thereof is administered in combination with XENP32803. In some embodiments, mezagitamab (TAK-079) or an antigen-binding fragment thereof is administered in combination with XENP24306 and XENP32803. In some embodiments, felzartamab (MOR202) is administered in combination with XENP24306. In some embodiments, felzartamab (MOR202) is administered in combination with XENP32803. In some embodiments, felzartamab (MOR202) is administered in combination with XENP24306 and XENP32803. In some embodiments, felzartamab (MOR202) or an antigen-binding fragment thereof is administered in combination with XENP24306. In some embodiments, felzartamab (MOR202) or an antigen-binding fragment thereof is administered in combination with XENP32803. In some embodiments, felzartamab (MOR202) or an antigen-binding fragment thereof is administered in combination with XENP24306 and XENP32803.

The anti-CD38 antibody or antigen-binding fragment thereof may be administered parenterally. In some embodiments, the parenteral administration is subcutaneous. In some embodiments, the parenteral administration is intravenous.

The amount of the anti-CD38 antibody or antigen-binding fragment thereof to be administered in combination with the heterodimeric protein of the disclosure (or combinations thereof) varies depending upon the manner of administration, the age and body weight of the patient, and the clinical symptoms of the cancer to be treated. In some embodiments, the anti-CD38 antibody or antigen-binding fragment thereof is administered at its approved dosage. In some embodiment, the anti-CD38 antibody or antigen-binding fragment thereof is administered below its approved dosage. A physician will be able to determine the adequate dosage of the anti-CD38 antibody or antigen-binding fragment thereof to administer in combination with the heterodimeric protein of the disclosure. In some embodiments, the dosage of the anti-CD38 antibody or antigen-binding fragment thereof is about 1800 mg/30,000 U recombinant human P1H20 hyaluronidase (rHuPH20). In some embodiments, the dosage of the anti-CD38 antibody or antigen-binding fragment thereof is about 1800 mg/30,000 U rHuPH20 every week. In some embodiments, the dosage of the anti-CD38 antibody or antigen-binding fragment thereof is about 1800 mg/30,000 U rHuPH20 every two weeks. In some embodiments, the dosage of the anti-CD38 antibody or antigen-binding fragment thereof is about 1800 mg/30,000 U rHuPH20 every three weeks. In some embodiments, the dosage of the anti-CD38 antibody or antigen-binding fragment thereof is about 1800 mg/30,000 U rHuPH20 every four weeks. In some embodiments, the dosage of the anti-CD38 antibody is about 1800 mg/30,000 U rHuPH20 every five weeks. In some embodiments, the dosage of the anti-CD38 antibody or antigen-binding fragment thereof is about 1800 mg every week. In some embodiments, the dosage of the anti-CD38 antibody or antigen-binding fragment thereof is about 1800 mg every two weeks. In some embodiments, the dosage of the anti-CD38 antibody or antigen-binding fragment thereof is about 1800 mg every three weeks. In some embodiments, the dosage of the anti-CD38 antibody or antigen-binding fragment thereof is about 1800 mg every four weeks. In some embodiments, the dosage of the anti-CD38 antibody or antigen-binding fragment thereof is about 1800 mg every five weeks.

The heterodimeric proteins disclosed herein, or combinations thereof, may be administered simultaneously or sequentially with the anti-CD38 antibody or antigen-binding fragment thereof. In some embodiments, the heterodimeric proteins disclosed herein, or combinations thereof, and an anti-CD38 antibody or antigen-binding fragment thereof are administered simultaneously. In some embodiments, the heterodimeric proteins disclosed herein, or combinations thereof, and an anti-CD38 antibody or antigen-binding fragment thereof are administered sequentially. In some embodiments, the anti-CD38 antibody or antigen-binding fragment thereof is administered after administering the heterodimeric protein (or combination thereof). In some embodiments, the anti-CD38 antibody or antigen-binding fragment thereof is administered before administering the heterodimeric protein (or combination thereof). In some embodiments, the heterodimeric proteins disclosed herein or combinations thereof and the anti-CD38 antibody or antigen-binding fragment thereof are administered in the same composition. In some embodiments, the heterodimeric proteins disclosed herein, or combinations thereof, are administered in a different composition than the anti-CD38 antibody or antigen-binding fragment thereof.

In some embodiments, the anti-CD38 antibody or antigen-binding fragment thereof is administered daily, i.e., every 24 hours. In some embodiments, the anti-CD38 antibody or antigen-binding fragment thereof is administered weekly, i.e., once per week (Q1W). In some embodiments, the anti-CD38 antibody or antigen-binding fragment thereof is administered once every two weeks, i.e., once every 14 days (Q2W). In some embodiments, the anti-CD38 antibody or antigen-binding fragment thereof is administered once every three weeks, i.e., once every 21 days (Q3W). In some embodiments, the anti-CD38 antibody or antigen-binding fragment thereof is administered once every four weeks, i.e., once every 28 days (Q4W). In some embodiments, the anti-CD38 antibody or antigen-binding fragment thereof is administered once every five weeks (Q5W). In some embodiments, the anti-CD38 antibody or antigen-binding fragment thereof is administered once every six weeks (Q6W). In some embodiments, the anti-CD38 antibody or antigen-binding fragment thereof is administered once every seven weeks (Q7W). In some embodiments, the anti-CD38 antibody or antigen-binding fragment thereof is administered once every eight weeks (Q8W). In some embodiments, the anti-CD38 antibody or antigen-binding fragment thereof is administered once every nine weeks (Q9W). In some embodiments, the anti-CD38 antibody or antigen-binding fragment thereof is administered once every ten weeks (Q10W). In some embodiments, the anti-CD38 antibody or antigen-binding fragment thereof is administered once every eleven weeks (Q11W). In some embodiments, the anti-CD38 antibody or antigen-binding fragment thereof is administered once every twelve weeks (Q12W). In some embodiments, the anti-CD38 antibody or antigen-binding fragment thereof is administered once every month. In some embodiments, the anti-CD38 antibody or antigen-binding fragment thereof is administered once every two months. In some embodiments, the anti-CD38 antibody or antigen-binding fragment thereof is administered once every three months. In some embodiments, the anti-CD38 antibody or antigen-binding fragment thereof is administered once every four months. In some embodiments, the anti-CD38 antibody or antigen-binding fragment thereof is administered once every five months. In some embodiments, the anti-CD38 antibody or antigen-binding fragment thereof is administered once every six months. In some embodiments, the anti-CD38 antibody or antigen-binding fragment thereof is administered once every seven months. In some embodiments, the anti-CD38 antibody or antigen-binding fragment thereof is administered once every eight months. In some embodiments, the −CD38 antibody or antigen-binding fragment thereof is administered once every nine months. In some embodiments, the anti-CD38 antibody or antigen-binding fragment thereof is administered once every ten months. In some embodiments, the anti-CD38 antibody or antigen-binding fragment thereof is administered once every eleven months. In some embodiments, the anti-CD38 antibody or antigen-binding fragment thereof is administered once every twelve months. In some embodiments, the anti-CD38 antibody or antigen-binding fragment thereof is administered once every year. In some embodiments, the anti-CD38 antibody or antigen-binding fragment thereof is administered subcutaneously according to the frequency disclosed herein.

In some embodiments, the anti-CD38 antibody or antigen-binding fragment thereof is administered at any of the above frequencies in one or more cycles. In some embodiments, the anti-CD38 antibody or antigen-binding fragment thereof is administered at any of the above frequencies in 1, 2, 3, 4, 5, 6, 7, 8, 9, 10, 11, 12, 13, 14, 15, 16, 17, 18, 19 or 20 cycles. In some embodiments, the anti-CD38 antibody or antigen-binding fragment thereof is administered at a frequency of Q1W in one or more cycles. In some embodiments, the anti-CD38 antibody or antigen-binding fragment thereof is administered at a frequency of Q1W in 1, 2, 3, 4, 5, 6, 7, 8, 9, 10, 11, 12, 13, 14, 15, 16, 17, 18, 19 or 20 cycles. In some embodiments, the anti-CD38 antibody or antigen-binding fragment thereof is administered at a frequency of Q2W in one or more cycles. In some embodiments, the anti-CD38 antibody or antigen-binding fragment thereof is administered at a frequency of Q1W for four cycles. In some embodiments, the anti-CD38 antibody or antigen-binding fragment thereof is administered at a frequency of Q2W in 1, 2, 3, 4, 5, 6, 7, 8, 9, 10, 11, 12, 13, 14, 15, 16, 17, 18, 19 or 20 cycles. In some embodiments, the anti-CD38 antibody is administered at a frequency of Q2W for eight cycles. In some embodiments, the anti-CD38 antibody or antigen-binding fragment thereof is administered at a frequency of Q3W in one or more cycles. In some embodiments, the anti-CD38 antibody or antigen-binding fragment thereof is administered at a frequency of Q3W in 1, 2, 3, 4, 5, 6, 7, 8, 9, 10, 11, 12, 13, 14, 15, 16, 17, 18, 19 or 20 cycles. In some embodiments, the anti-CD38 antibody or antigen-binding fragment thereof is administered at a frequency of Q4W in one or more cycles. In some embodiments, the anti-CD38 antibody or antigen-binding fragment thereof is administered at a frequency of Q4W in 1, 2, 3, 4, 5, 6, 7, 8, 9, 10, 11, 12, 13, 14, 15, 16, 17, 18, 19 or 20 cycles.

In some embodiments, the anti-CD38 antibody or antigen-binding fragment thereof is administered subcutaneously. In some embodiments, the anti-CD38 antibody or antigen-binding fragment thereof is administered subcutaneously at a fixed dose on Day 1 and 8 of each 14-day cycle. In some embodiments, the anti-CD38 antibody or antigen-binding fragment thereof is administered subcutaneously at a fixed dose on Day 1 of each 14-day cycle. In some embodiments, the anti-CD38 antibody or antigen-binding fragment thereof is administered subcutaneously at a fixed dose on Day 1 of each 28-day cycle. In some embodiments, the heterodimeric protein of the disclosure (or combinations thereof) is administered by intravenous infusion at a fixed dose on Day 1 of each 14-day cycle. In some embodiments, the heterodimeric protein of the disclosure (or combinations thereof) is administered by intravenous infusion at a fixed dose on Day 2 of each 14-day cycle. In some embodiments, the heterodimeric protein of the disclosure (or combinations thereof) is administered by intravenous infusion at a fixed dose on Day 1 of each 28-day cycle. In some embodiments, the heterodimeric protein of the disclosure (or combinations thereof) is administered by intravenous infusion at a fixed dose on Day 2 of each 28-day cycle.

In some embodiments, the anti-CD38 antibody or antigen-binding fragment thereof is administered subcutaneously at a fixed dose on Day 1 and 8 of each 14-day cycle in combination with the heterodimeric protein of the disclosure. In some embodiments, the anti-CD38 antibody or antigen-binding fragment thereof is administered subcutaneously at a fixed dose on Day 1 and 8 of each 14-day cycle and is administered sequentially with the heterodimeric protein of the disclosure. In some embodiments, the anti-CD38 antibody or antigen-binding fragment thereof is administered subcutaneously at a fixed dose on Day 1 and 8 of each 14-day cycle and is administered simultaneously with the heterodimeric protein of the disclosure. In some embodiments, the anti-CD38 antibody or antigen-binding fragment thereof is administered subcutaneously at a fixed dose on Day 1 and 8 of each 14-day cycle in combination with the heterodimeric protein of the disclosure that is administered intravenously on Day 1 of each 14-day cycle. In some embodiments, the anti-CD38 antibody or antigen-binding fragment thereof is administered subcutaneously at a fixed dose on Day 1 and 8 of each 14-day cycle in combination with the heterodimeric protein of the disclosure that is administered intravenously on Day 2 of each 14-day cycle. In some embodiments, the anti-CD38 antibody or antigen-binding fragment thereof is administered subcutaneously at a fixed dose on Day 1 of each 14-day cycle in combination with the heterodimeric protein of the disclosure that is administered intravenously on Day 1 of each 14-day cycle. In some embodiments, the anti-CD38 antibody or antigen-binding fragment thereof is administered subcutaneously at a fixed dose on Day 1 of each 14-day cycle in combination with the heterodimeric protein of the disclosure that is administered intravenously on Day 2 of each 14-day cycle. In some embodiments, the anti-CD38 antibody or antigen-binding fragment thereof is administered subcutaneously at a fixed dose on Day 1 of each 28-day cycle in combination with the heterodimeric protein of the disclosure that is administered intravenously on Day 1 of each 28-day cycle. In some embodiments, the anti-CD38 antibody or antigen-binding fragment thereof is administered subcutaneously at a fixed dose on Day 1 of each 28-day cycle in combination with the heterodimeric protein of the disclosure that is administered intravenously on Day 2 of each 28-day cycle.

In some embodiments, the heterodimeric protein is administered at a frequency of Q2W, and the anti-CD38 antibody or antigen-binding fragment thereof is administered at a frequency of Q1W in one or more cycles. In some embodiments, the heterodimeric protein is administered at a frequency of Q2W, and the anti-CD38 antibody or antigen-binding fragment thereof is administered at a frequency of Q2W in one or more cycles. In some embodiments, the heterodimeric protein is administered at a frequency of Q4W, and the anti-CD38 antibody or antigen-binding fragment thereof is administered at a frequency of Q4W in one or more cycles.

In some embodiments, the anti-CD38 antibody or antigen-binding fragment thereof is administered at a dose of about 1800 mg on day 1 of each 14-day cycle. In some embodiments, the anti-CD38 antibody is administered at a dose of about 1800 mg on day 1 and 8 of each 14-day cycle. In some embodiments, the anti-CD38 antibody or antigen-binding fragment thereof is administered at a dose of about 1800 mg on day 1 of each 28-day cycle. In some embodiments, the anti-CD38 antibody or antigen-binding fragment thereof is administered at a dose of about 1800 mg on day 1 of each 14-day cycle in combination with the heterodimeric protein of the disclosure. In some embodiments, the anti-CD38 antibody or antigen-binding fragment thereof is administered at a dose of about 1800 mg on day 1 and 8 of each 14-day cycle in combination with the heterodimeric protein of the disclosure. In some embodiments, the anti-CD38 antibody or antigen-binding fragment thereof is administered at a dose of about 1800 mg on day 1 of each 28-day cycle in combination with the heterodimeric protein of the disclosure. In some embodiments, the anti-CD38 antibody or antigen-binding fragment thereof is administered using the approved dosage regimen.

In some embodiments, the anti-CD38 antibody is an established therapy for the cancer and addition of the heterodimeric protein treatment to the regimen improves the therapeutic benefit to the patients. Such improvement could be measured as increased responses on a per patient basis or increased responses in the patient population. The heterodimeric proteins disclosed herein or combinations thereof and the anti-CD38 antibody or antigen-binding fragment thereof may synergize. In some embodiments, the heterodimeric proteins disclosed herein, or combinations thereof, may be administered at a dosage less than its therapeutically effective dose when administered as a monotherapy. In some embodiments, the anti-CD38 antibody or antigen-binding fragment thereof may be administered at a dosage less than its therapeutically effective dose when administered as a monotherapy.

In some embodiments, the subject has been previously administered an agent for the treatment of the blood cancer. In some embodiments, the subject has been previously administered one or more prior treatments for treatment of the blood cancer. In some embodiments, the subject has been previously administered one prior treatment. In some embodiments, the subject has been previously administered two prior treatments. In some embodiments, the subject has been previously administered three prior treatments. In some embodiments, the subject has been previously administered four prior treatments. In some embodiments, the subject has been previously administered five prior treatments. In some embodiments, the prior treatment administered to the subject is an immunomodulatory drug, a proteasome inhibitor, an anti-CD38 monoclonal antibody, or a combination thereof. In some embodiments, the prior treatment administered to the subject is an immunomodulatory drug. In some embodiments, the immunomodulatory drug is selected from the group consisting of lenalidomide, thalidomide, and pomalidomide. In some embodiments, the immunomodulatory drug is lenalidomide. In some embodiments, the immunomodulatory drug is thalidomide. In some embodiments, the immunomodulatory drug is pomalidomide. In some embodiments, the prior treatment administered to the subject is a proteasome inhibitor. In some embodiments, the proteasome inhibitor is selected from the group consisting of bortezomib, carfilzomib, and ixazomib. In some embodiments, the proteasome inhibitor is selected from the group consisting of bortezomib. In some embodiments, the proteasome inhibitor is selected from the group consisting of carfilzomib. In some embodiments, the proteasome inhibitor is selected from the group consisting of ixazomib. In some embodiments, the prior treatment administered to the subject is an anti-CD38 monoclonal antibody. In some embodiments, the anti-CD38 monoclonal antibody is selected from the group consisting of daratumumab, isatuximab, mezagitamab (TAK-079) and felzartamab (MOR202). In some embodiments, the anti-CD38 monoclonal antibody is daratumumab. In some embodiments, the anti-CD38 monoclonal antibody is isatuximab. In some embodiments, the anti-CD38 monoclonal antibody is mezagitamab. In some embodiments, the anti-CD38 monoclonal antibody is felzartamab.

Examples of blood cancers to be treated by the combination of the heterodimeric proteins of the disclosure the anti-CD38 antibody or antigen-binding fragment thereof include, but are not limited, to leukemias, lymphomas, and myelomas. More particular non-limiting examples of such blood cancers include acute myeloid leukemia, adult acute lymphoblastic leukemia, chronic lymphocytic leukemia, non-Hodgkin's lymphoma, B-cell non-Hodgkin's lymphoma, and multiple myeloma. In some embodiments, the blood cancer is a relapsed or refractory. In some embodiments, the blood cancer is selected from the group consisting of leukemia, acute myeloid leukemia, adult acute lymphoblastic leukemia, chronic lymphocytic leukemia, lymphoma, non-Hodgkin's lymphoma, B-cell non-Hodgkin's lymphoma, and multiple myeloma. In some embodiments, the blood cancer is selected from the group consisting of leukemia, acute myeloid leukemia, adult acute lymphoblastic leukemia, chronic lymphocytic leukemia. In some embodiments, the blood cancer is selected from the group consisting of lymphoma, non-Hodgkin's lymphoma, B-cell non-Hodgkin's lymphoma. In some embodiments, the blood cancer is leukemia. In some embodiments, the blood cancer is acute myeloid leukemia. In some embodiments, the blood cancer is adult acute lymphoblastic leukemia. In some embodiments, the blood cancer is chronic lymphocytic leukemia. In some embodiments, the blood cancer is lymphoma. In some embodiments, the blood cancer is non-Hodgkin's lymphoma. In some embodiments, the blood cancer is B-cell non-Hodgkin's lymphoma. In some embodiments, the blood cancer is multiple myeloma. In some embodiments, the blood cancer is relapsed or refractory multiple myeloma. In some embodiments, the blood cancer is a blood cancer for which standard therapy does not exist, has proven to be ineffective or intolerable, or is considered inappropriate, or for whom a clinical trial of an investigational agent is a recognized standard of care.

A combination therapy could also provide improved responses at lower or less frequent doses of the anti-CD38 antibody or antigen-binding fragment thereof resulting in a better tolerated treatment regimen. For example, the combined therapy of the heterodimeric protein(s) and the anti-CD38 antibody or antigen-binding fragment thereof could provide enhanced clinical activity through various mechanisms, including augmented ADCC, ADCP, and/or NK cell, T cell, neutrophil or monocytic cell levels or immune responses.

### Exemplary Embodiments

Particular embodiments of the disclosure are set forth in the following numbered embodiments:
1. A method of treating a blood cancer in a subject in need thereof, the method comprising administering to the subject a therapeutically effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.
2. A method for inducing the proliferation of CD8⁺ effector memory T cells in a subject suffering from a blood cancer, the method comprising administering to the subject an effective amount of a heterodimeric protein, wherein the heterodimeric protein comprises (i) a first monomer comprising IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.
3. A method for inducing the proliferation of NK cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.
4. A method for inducing the proliferation of CD8⁺ effector memory T cells and NK cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.
5. A method for inducing IFNγ production in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.
6. The method according to any one of embodiments 1-5, wherein each of said first and second Fc domains comprises amino acid substitutions E233P, L234V, L235A, G236del, and S267K, according to EU numbering.
7. The method according to any one of embodiments 1-6, wherein said first Fc domain further comprises amino acid substitutions L368D and K370S and said second Fc domain further comprises amino acid substitutions S364K and E357Q, according to EU numbering.
8. The method according to any one of embodiments 1-6, wherein said first Fc domain further comprises amino acid substitutions S364K and E357Q and said second Fc domain further comprises amino acid substitutions L368D and K370S, according to EU numbering.
9. The method according to any one of embodiments 1-8, wherein said first Fc domain further comprises amino acid substitutions Q295E, N384D, Q418E and N421D, according to EU numbering.
10. The method according to any one of embodiments 1-8, wherein said second Fc domain further comprises amino acid substitutions Q295E, N384D, Q418E and N421D, according to EU numbering.
11. The method according to any one of embodiments 1-10, wherein said second Fc domain further comprises amino acid substitution K246T, according to EU numbering.
12. The method according to any one of embodiments 1-11, wherein said IL-15 protein comprises amino acid substitutions D30N, E64Q and N65D.
13. The method according to any one of embodiments 1-12, wherein said IL-15 protein comprises the amino acid sequence set forth in SEQ ID NO: 5.
14. The method according to any one of embodiments 1-13, wherein said sushi domain of IL-15Rα protein comprises the amino acid sequence set forth in SEQ ID NO: 4.
15. The method according to any one of embodiments 1-14, wherein the IL-15 protein is covalently attached to the N-terminus of the first Fc domain via a first linker.
16. The method according to any one of embodiments 1-15, wherein the IL-15Rα protein is covalently attached to the N-terminus of the second Fc domain via a second linker.
17. The method according to any one of embodiments 1-16, wherein the IL-15 protein is covalently attached to the N-terminus of the first Fc domain via a first linker and the IL-15Rα protein is covalently attached to the N-terminus of the second Fc domain via a second linker.
18. The method according to any one of embodiments 15-17, wherein the first linker and/or second linker is independently a variable length Gly-Ser linker.
19. The method according to embodiment 18, wherein the first linker and/or the second linker independently comprises a linker selected from the group consisting of (Gly-Gly-Gly-Gly-Ser)n (SEQ ID NO: 39), (Ser-Ser-Ser-Ser-Gly)n (SEQ ID NO: 40), (Gly-Ser-Ser-Gly-Gly)n (SEQ ID NO: 41), and (Gly-Gly-Ser-Gly-Gly)n (SEQ ID NO: 42), where n is an integer between 1 and 5.
20. A method of treating a blood cancer in a subject in need thereof, the method comprising administering to the subject a therapeutically effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains comprises a set of amino acid substitutions selected from the group consisting of S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L; K370S: S364K/E357Q; S267K/S364K/E357Q: S267K/L368D/K370S; L368D/K370S: S364K/E357Q; S364K: L368D/K370S; S364K: L368E/K370S; D401K: T411E/K360E/Q362E; S364K/E357L: L368D/K370S; and S364K/E357Q: K370S, according to EU numbering.
21. A method for inducing the proliferation of CD8+ effector memory T cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains comprises a set of amino acid substitutions selected from the group consisting of S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L; K370S: S364K/E357Q; S267K/S364K/E357Q: S267K/L368D/K370S; L368D/K370S: S364K/E357Q; S364K: L368D/K370S; S364K: L368E/K370S; D401K: T411E/K360E/Q362E; S364K/E357L: L368D/K370S; and S364K/E357Q: K370S, according to EU numbering.
22. A method for inducing the proliferation of NK cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fe domains comprises a set of amino acid substitutions selected from the group consisting of S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L; K370S: S364K/E357Q; S267K/S364K/E357Q: S267K/L368D/K370S; L368D/K370S: S364K/E357Q; S364K: L368D/K370S; S364K: L368E/K370S; D401K: T411E/K360E/Q362E; S364K/E357L: L368D/K370S; and S364K/E357Q: K370S, according to EU numbering.
23. A method for inducing the proliferation of CD8⁺ effector memory T cells and NK cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains comprises a set of amino acid substitutions selected from the group consisting of S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L; K370S: S364K/E357Q; S267K/S364K/E357Q: S267K/L368D/K370S; L368D/K370S: S364K/E357Q; S364K: L368D/K370S; S364K: L368E/K370S; D401K: T411E/K360E/Q362E; S364K/E357L: L368D/K370S; and S364K/E357Q: K370S, according to EU numbering.
24. A method for inducing IFNγ production in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising an IL-15Rα protein and a second Fc domain, wherein said IL-15Rα protein is covalently attached to the N-terminus of said second Fc domain; wherein said first and said second Fc domains comprises a set of amino acid substitutions selected from the group consisting of S267K/L368D/K370S: S267K/S364K/E357Q; S364K/E357Q: L368D/K370S; L368D/K370S: S364K; L368E/K370S: S364K; T411E/K360E/Q362E: D401K; L368D/K370S: S364K/E357L; K370S: S364K/E357Q; S267K/S364K/E357Q: S267K/L368D/K370S; L368D/K370S: S364K/E357Q; S364K: L368D/K370S; S364K: L368E/K370S; D401K: T411E/K360E/Q362E; S364K/E357L: L368D/K370S; and S364K/E357Q: K370S, according to EU numbering.
25. The method according to any one of embodiments 20-24, wherein each of said first and/or second Fc domains independently further comprises amino acid substitutions Q295E, N384D, Q418E and N42ID, according to EU numbering.
26. The method according to any one of embodiments 20-25, wherein each of said first and/or second Fc domains independently further comprises amino acid substitutions selected from the group consisting of G236R/L328R; E233P/L234V/L235A/G236del/S239K; E233P/L234V/L235A/G236del/S267K; E233P/L234V/L235A/G236del/S239K/A327G; E233P/L234V/L235A/G236del/S267K/A327G; and E233P/L234V/L235A/G236del, according to EU numbering and wherein the Fc domains are derived from IgG1 or IgG3 Fc domains.
27. The method according to any one of embodiments 20-25, wherein each of said first and/or second Fc domains independently further comprises amino acid substitutions selected from the group consisting of L328R; S239K; and S267K, according to EU numbering and wherein the Fc domains are derived from IgG2 Fc domain.
28. The method according to any one of embodiments 20-25, wherein each of said first and/or second Fc domains independently further comprises amino acid substitutions selected from the group consisting of G236R/L328R; E233P/F234V/L235A/G236del/S239K; E233P/F234V/L235A/G236del/S267K; E233P/F234V/L235A/G236del/S239K/A327G; E233P/F234V/L235A/G236del/S267K/A327G; and E233P/F234V/L235A/G236del, according to EU numbering and wherein the Fc domains are derived from IgG4 Fc domain.
29. The method according to any one of embodiments 20-28, wherein said IL-15 protein comprises one or more amino acid substitutions selected from the group consisting of N1D, N4D, D8N, D30N, D61N, E64Q, N65D and Q108E.
30. The method according to any one of embodiments 20-28, wherein said IL-15 protein and said IL-15Rα protein comprise a set of amino acid substitutions or additions selected from E87C: 65DPC; E87C: 65DCA; V49C: S40C; L52C: S40C; E89C: K34C; Q48C: G38C; E53C: L42C; C42S: A37C and L45C: A37C, respectively.
31. The method according to any one of embodiments 20-30, wherein said IL-15 protein comprises a polypeptide sequence selected from the group consisting of SEQ ID NO:1 and SEQ ID NO:2.
32. The method according to any one of embodiments 20-31, wherein said IL-15Rα protein comprises a polypeptide sequence selected from the group consisting of SEQ ID NO:3 and SEQ ID NO:4.
33. The method according to any one of embodiments 20-24, wherein the first Fc domain comprises amino acid substitutions L368D and K370S; wherein the second Fc domain further comprises amino acid substitutions S364K and E357Q; and wherein each of said first and second Fc domains further comprises amino acid substitutions C220S, E233P, L234V, L235A, G236del, S267K, M428L and N434S, according to EU numbering; wherein said IL-15 protein comprises amino acid substitutions D30N, E64Q and N65D; and wherein said IL-15Rα protein comprises SEQ ID NO:4.
34. The method according to any one of embodiments 20-24, wherein the first Fc domain comprises amino acid substitutions S364K and E357Q; wherein the second Fc domain comprises amino acid substitutions L368D and K370S; and wherein each of said first and second Fc domains further comprises amino acid substitutions C220S, E233P, L234V, L235A, G236del, S267K, M428L and N434S, according to EU numbering; wherein said IL-15 protein comprises amino acid substitutions D30N, E64Q and N65D; and wherein said IL-15Rα protein comprises SEQ ID NO:4.
35. The method according to any one of embodiments 20-24, wherein the first Fc domain comprises amino acid substitutions L368D and K370S; wherein the second Fc domain comprises amino acid substitutions K246T, S364K and E357Q; and wherein each of said first and second Fc domains further comprises amino acid substitutions C220S, E233P, L234V, L235A, G236del, S267K, M428L and N434S, according to EU numbering; wherein said IL-15 protein comprises amino acid substitutions D30N, E64Q and N65D; and wherein said IL-15Rα protein comprises SEQ ID NO:4.
36. The method according to any one of embodiments 20-24, wherein the first Fc domain comprises amino acid substitutions S364K and E357Q; wherein the second Fc domain comprises amino acid substitutions K246T, L368D and K370S; and wherein each of said first and second Fc domains further comprises amino acid substitutions C220S, E233P, L234V, L235A, G236del, S267K, M428L and N434S, according to EU numbering; wherein said IL-15 protein comprises amino acid substitutions D30N, E64Q and N65D; and wherein said IL-15Rα protein comprises SEQ ID NO:4.
37. The method according to any one of embodiments 20-36, wherein the IL-15 protein is covalently attached to the N-terminus of the first Fc domain via a first linker.
38. The method according to any one of embodiments 20-37, wherein the IL-15Rα protein is covalently attached to the N-terminus of the second Fc domain via a second linker.
39. The method according to any one of embodiments 20-38, wherein the IL-15 protein is covalently attached to the N-terminus of the first Fc domain via a first linker and the IL-15Rα protein is covalently attached to the N-terminus of the second Fc domain via a second linker.
40. The method according to any one of embodiments 37-39, wherein the first linker and/or second linker is independently a variable length Gly-Ser linker.
41. The method according to embodiment 40, wherein the first linker and/or the second linker independently comprises a linker selected from the group consisting of (Gly-Gly-Gly-Gly-Ser)n (SEQ ID NO: 39), (Ser-Ser-Ser-Ser-Gly)n (SEQ ID NO: 40), (Gly-Ser-Ser-Gly-Gly)n (SEQ ID NO: 41), and (Gly-Gly-Ser-Gly-Gly)n (SEQ ID NO: 42), where n is an integer between 1 and 5.
42. The method according to any one of embodiments 20-41, wherein said heterodimeric protein is selected from the group consisting of XENP22822, XENP23504, XENP24045, XENP24306, XENP22821, XENP23343, XENP23557, XENP24113, XENP24051, XENP24341, XENP24052, XENP24301, and XENP32803 proteins.
43. The method according to any one of embodiments 1-5 and 20-24, wherein said first monomer comprises the amino acid sequence set forth in SEQ ID NO: 9, and the second monomer comprises the amino acid sequence set forth in SEQ ID NO: 10.
44. The method according to any one of embodiments 1-5 and 20-24, wherein said first monomer comprises the amino acid sequence set forth in SEQ ID NO: 9, and the second monomer comprises the amino acid sequence set forth in SEQ ID NO: 16.
45. The method according to any one of embodiments 1-5 and 20-24, wherein said heterodimeric protein is XENP24306, XENP32803, or a combination thereof.
46. The method according to any one of embodiments 1-45, wherein a combination of a first heterodimeric protein and a second heterodimeric protein is administered to the subject.
47. The method according to embodiment 46, wherein the first heterodimeric protein comprises a first monomer comprising the amino acid sequence set forth in SEQ ID NO: 9, and a second monomer comprising the amino acid sequence set forth in SEQ ID NO: 10; and the second heterodimeric protein comprises a first monomer comprising the amino acid sequence set forth in SEQ ID NO: 9, and a second monomer comprising the amino acid sequence set forth in SEQ ID NO: 16.
48. The method according to embodiment 46 or 47, wherein said first and second heterodimeric proteins are administered simultaneously.
49. The method according to embodiment 46 or 47, wherein said first and second heterodimeric proteins are administered sequentially.
50. The method according any one of embodiments 1-49, wherein said blood cancer is selected from the group consisting of leukemia, acute myeloid leukemia, adult acute lymphoblastic leukemia, chronic lymphocytic leukemia, lymphoma, non-Hodgkin's lymphoma, B-cell non-Hodgkin's lymphoma, and multiple myeloma.
51. The method according to embodiment 50, wherein said blood cancer is multiple myeloma.
52. The method according to embodiment 51, wherein said multiple myeloma is relapsed or refractory multiple myeloma.
53. The method according to embodiment 50, wherein said blood cancer is B-cell non-Hodgkin's lymphoma.
54. The method according to embodiment 50, wherein said blood cancer is chronic lymphocytic leukemia.
55. The method according to any one of embodiments 1-54, wherein the subject has been previously administered one or more prior treatments.
56. The method according to embodiment 55, wherein the prior treatment is an immunomodulatory drug, a proteasome inhibitor, or an anti-CD38 monoclonal antibody.
57. The method according to embodiment 56, wherein the immunomodulatory drug is selected from the group consisting of lenalidomide, thalidomide, and pomalidomide.
58. The method according to embodiment 56, wherein the proteasome inhibitor is selected from the group consisting of bortezomib, carfilzomib, and ixazomib.
59. The method according to embodiment 56, wherein the anti-CD38 monoclonal antibody is selected from the group consisting of daratumumab, isatuximab, mezagitamab, and felzartamab.
60. The method according to any one of embodiments 1-59, wherein said heterodimeric protein or combination of heterodimeric proteins is administered at a dose selected from the group consisting of about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.1 mg/kg, about 0.12 mg/kg, about 0.16 mg/kg, about 0.2 mg/kg, about 0.24 mg/kg and about 0.32 mg/kg body weight.
61. The method according to embodiment 60, wherein said heterodimeric protein or combination of heterodimeric proteins is administered at a dose selected from the group consisting of about 0.01 mg/kg, about 0.02 mg/kg, about 0.04 mg/kg, and about 0.06 mg/kg body weight.
62. The method according to any one of embodiments 1-61, wherein said heterodimeric protein or combination of heterodimeric proteins is administered at a dose selected from the group consisting of 0.0025 mg/kg, 0.005 mg/kg, 0.01 mg/kg, 0.015 mg/kg, 0.02 mg/kg, 0.025 mg/kg, 0.03 mg/kg, 0.04 mg/kg, 0.05 mg/kg, 0.06 mg/kg, 0.08 mg/kg, 0.10 mg/kg, 0.16 mg/kg, 0.20 mg/kg, 0.24 mg/kg and 0.32 mg/kg body weight.
63. The method according to embodiment 62, wherein said heterodimeric protein or combination of heterodimeric proteins is administered at a dose selected from the group consisting of 0.01 mg/kg, 0.02 mg/kg, 0.04 mg/kg, and 0.06 mg/kg body weight.
64. The method according to any one of embodiments 1-63, wherein said method further comprises administering to the subject an anti-CD38 monoclonal antibody.
65. The method according to embodiment 64, wherein the anti-CD38 monoclonal antibody is selected from the group consisting of daratumumab, isatuximab, mezagitamab, and felzartamab.
66. The method according to embodiment 65, wherein the anti-CD38 monoclonal antibody is daratumumab.
67. The method according to any one of embodiments 64-66, wherein said heterodimeric protein and said anti-CD38 monoclonal antibody are administered simultaneously.
68. The method according to any one of embodiments 64-66, wherein said heterodimeric protein and said anti-CD38 monoclonal antibody are administered sequentially.
69. The method according to any one of embodiments 1-68, wherein said heterodimeric protein is administered at a frequency selected from the group consisting of Q1W, Q2W, Q3W, Q4W, Q5W and Q6W.
70. The method according to embodiment 69, wherein said heterodimeric protein is administered at a frequency of Q1W in one or more cycles.
71. The method according to embodiment 69, wherein said heterodimeric protein is administered at a frequency of Q2W in one or more cycles.
72. The method according to embodiment 69, wherein said heterodimeric protein is administered at a frequency of Q4W in one or more cycles.
73. The method according to any one of embodiments 64-72, wherein said anti-CD38 monoclonal antibody is administered at a frequency selected from the group consisting of Q1W, Q2W, Q3W, Q4W, Q5W and Q6W.
74. The method according to embodiment 73, wherein said anti-CD38 monoclonal antibody is administered at a frequency of Q1W in one or more cycles.
75. The method according to embodiment 73, wherein said anti-CD38 monoclonal antibody is administered at a frequency of Q2W in one or more cycles.
76. The method according to embodiment 73, wherein said anti-CD38 monoclonal antibody is administered at a frequency of Q4W in one or more cycles.
77. The method according to embodiment 73, wherein said heterodimeric protein is administered at a frequency of Q2W, and wherein said anti-CD38 monoclonal antibody is administered at a frequency of Q1W in one or more cycles.
78. The method according to embodiment 73, wherein said heterodimeric protein is administered at a frequency of Q2W, and wherein said anti-CD38 monoclonal antibody is administered at a frequency of Q2W in one or more cycles.
79. The method according to embodiment 73, wherein said heterodimeric protein is administered at a frequency of Q4W, and wherein said anti-CD38 monoclonal antibody is administered at a frequency of Q4W in one or more cycles.
80. The method according to any one of embodiments 1-79, wherein said heterodimeric protein is administered intravenously.
81. The method according to any one of embodiments 64-68, wherein said anti-CD38 monoclonal antibody is administered subcutaneously.

### EXAMPLES

### Example 1: Non-Clinical Pharmacology of XENP24306

As detailed below, a combination of IL15/IL15Rα heterodimeric proteins (XENP24306 (˜82%) and XENP32803 (˜18%) (“XENP24306+XENP32803”)) was evaluated in multiple in vitro and in vivo studies to characterize non-clinical pharmacology properties. In vitro studies demonstrated that the combination of IL15/IL15Rα heterodimeric proteins showed binding to human and cynomolgus IL-2/IL-15@7 receptor complex (CD122/CD132), had activity in human and cynomolgus CD8⁺ T cells and NK cells, but was inactive in rodent cells (mouse and rat). XENP24306+XENP32803 showed increased neonatal Fc receptor (FcRn) binding (at pH 6.0) but had no effector function in terms of mediating antibody-dependent cellular cytotoxicity (ADCC) or complement-dependent cytotoxicity (CDC). Both in vitro and in vivo studies showed that XENP24306+XENP32803 preferably expanded CD8⁺ T cells and NK cells, with modest impact on expansion of CD4⁺ T-helper lymphocytes, while having minimal impact on expansion of the Treg population and cytokine release syndrome (CRS)-associated cytokines.

### In Vitro Studies

The IL-15 component of XENP24306 and XENP32803 comprises three amino acid substitutions (D30N, E64Q, and N65D). These substitutions result in reduced potency of IL-15. The binding affinity XENP24306+XENP32803 to human and cynomolgus monkey IL-2/IL-15 βγ receptor complex (CD122/CD132) was determined with surface plasmon resonance. Similar binding kinetics and affinities were observed between the two species, establishing the relevancy of cynomolgus monkey as a preclinical animal species for pharmacology and toxicity studies.

XENP24306 and XENP32803 are effectorless, demonstrated by lack of binding to FcγR and human complement component 1q (C1q), and are not expected to induce target-cell killing via ADCC or CDC mechanisms. Specifically, the Fc region XENP24306 and XENP32803 was engineered to remove binding to human, cynomolgus monkey, and mouse FcγR; no binding interactions were detected with the Bio-Layer Interferometry (BLI) method. Binding of XENP24306+XENP32803 to human C1q, a critical component of the C1 complex that initiates the complement system, was also assessed using BLI, and no binding was observed.

Furthermore, the Fc regions of XENP24306 and XENP32803 were engineered to enhance binding to FcRn at a lower pH (6.0) with the goal of extending the half-life of XENP24306. Binding interactions with human, cynomolgus monkey, and mouse FcRn were determined with the BLI method, and affinities of XENP24306+XENP32803 for these receptors were significantly enhanced at pH 6.0, the physiologically relevant pH for endosome trafficking.

XENP24306+XENP32803-species selectivity was evaluated using a phospho-STAT5 assay. Binding of IL-15/IL-15Rα receptor complex to CD122/CD132-expressing lymphocytes led to activation of the Janus kinase signal transducer and activator of transcription signaling pathway, which resulted in phosphorylation of STAT5 and subsequent cell proliferation. XENP24306+XENP32803 did not induce phosphorylation of STAT5 in mouse or rat CD8⁺ T cells, which thereby precluded use of rodents for toxicity studies or the use of syngeneic mouse models for evaluation of XENP24306+XENP32803 for antitumor efficacy.

Potency of XENP24306+XENP32803 was assessed in in vitro cell proliferation assays. Human CD8⁺ T cells and NK cells showed strong proliferative responses to XENP24306+XENP32803 treatment. Among these two target cell populations, XENP24306+XENP32803 showed relatively higher potency for NK-cell (half maximal effective concentration [EC₅₀]: 1.2 μg/mL) than CD8⁺ T cell (EC₅₀: 12.7 μg/mL) proliferation (FIGS. 1A and 1B). In addition to CD8⁺ T cell and NK-cell proliferation, XENP24306+XENP32803 also induced IFNγ production in human PBMCs. XENP24306+XENP32803 also promoted NK-cell (EC₅₀: 0.5 μg/mL) and CD8⁺ T cell (EC₅₀: 3.8 μg/mL) proliferation in cynomolgus monkey PBMCs, which validated cynomolgus monkey as a nonclinical animal species for pharmacology and toxicity studies.

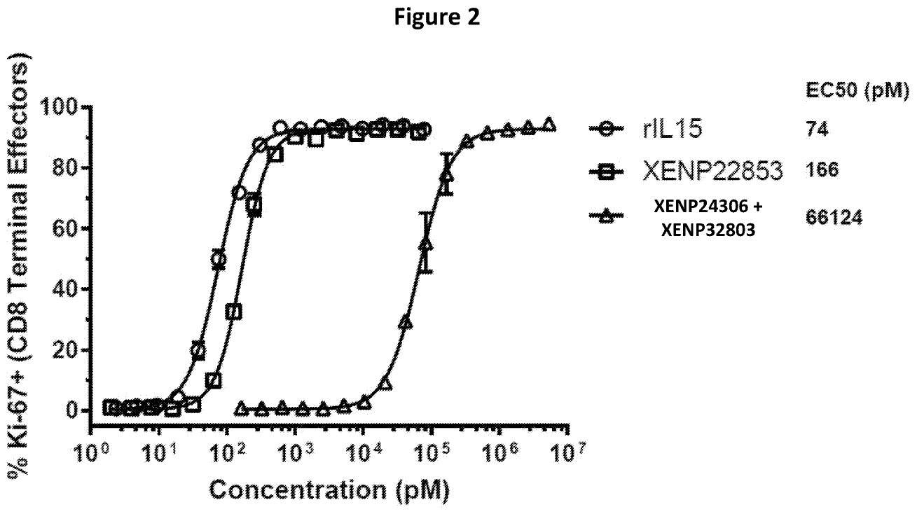

XENP24306 and XENP32803 are potency-reduced, recombinant human IL-15s, designed as IL-15/IL-15Rα heterodimer Fe fusion proteins. Approximately 900-fold lower potency was observed for XENP24306+XENP32803 than recombinant wild-type IL-15 and approximately 400-fold lower potency than recombinant wild-type IL-15 (rIL15) of similar format (wild-type IL-15/wild-type IL-15Rα heterodimer Fe fusion; named as XENP22853; SEQ ID NO: 11 (wild-type IL-15-Fc first monomer) and SEQ ID NO: 7 (IL-15Rα-Fc second monomer)), as shown on CD8⁺ terminal effector T cells (FIG. 2). XENP24306+XENP32803 potency was assessed on different human immune cell subsets. Specifically, Human PBMC were treated with increasing concentrations of XENP24306+XENP32803, recombinant wild-type IL15, or wild-type IL-15/wild-type IL-15Rα heterodimer Fc fusion (XENP22853) for 4 days and assayed by flow cytometry for proliferation through intracellular staining for the cell cycle protein Ki67. FIG. 2 shows results for CD8⁺ terminal effector T cells defined by gating for CD3⁺ CD8⁺CD45RA⁺ CCR7⁻ CD28⁻ CD95⁺ population. Curve fits were generated using the least squares method. EC₅₀ values were determined by non-linear regression analysis using agonist versus response and a variable-slope (four-parameter) equation. XENP24306+XENP32803 enhanced activation of effector memory CD8⁺ and CD4⁺ T cells and NK cells as indicated by increased frequencies of these cell subsets expressing the cell proliferation marker Ki67 and cell activation markers CD69 and CD25. XENP24306 had minimal effects on naïve CD8⁺ or CD4⁺ T cells.

Two additional in vitro toxicity studies were performed (1) an assessment of the binding profile of XENP24306+XENP32803 using a human plasma membrane protein cell array and (2) an assessment of cytokine release induced by XENP24306+XENP32803, which compared the ability of soluble and immobilized XENP24306+XENP32803 to induce cytokine production. Data from multiple experiments using an optimized concentration of XENP24306+XENP32803 (20 μg/mL) showed that there were no convincing off-target binding interactions identified for XENP24306+XENP32803. Potential risk of Cytokine release syndrome (CRS) with XENP24306+XENP32803 was investigated using unstimulated human PBMCs in vitro. To evaluate the potential for XENP24306+XENP32803 to induce production of cytokines associated with CRS, in vitro stimulation of human PBMCs was performed at 10 and 20 μg/mL (43-fold and 87-fold higher than predicted Cmax (0.23 μg/mL) in blood at the recommended FIH dose (0.01 mg/kg)) concentrations of XENP24306+XENP32803. Both immobilized and soluble formats of XENP24306+XENP32803 induced IFNγ production. The magnitude of IFNγ induction with XENP24306 (9- to 14-fold compared to vehicle control) was multi-fold lower than observed with an anti-CD28 antibody (393-fold compared to vehicle control) or anti-CD3 antibody (1605-fold compared to vehicle control), used as positive controls. No induction of any other cytokines such as IL-1α, IL-2, IL-4, IL-6, IL-8, IL-10, IL-12p70, IL-13, or TNF was observed. XENP24306+XENP32803 did not induce inflammatory cytokines that were known to be involved in CRS, such as IL-6 and TNF, which indicates that the risk of XENP24306+XENP32803 inducing CRS is low.

### In Vivo Studies

Immune responses were assessed in cynomolgus monkeys following single or repeat doses of XENP24306+XENP32803. No apparent elevation of inflammatory cytokines, such as IL-6, tumor necrosis factor-α (TNFα), and IFNγ was observed following IV doses of XENP24306+XENP32803. Transient elevation of other cytokines and chemokines, such as IP-10, MCP-1 (monocyte chemoattractant protein-1), MIP-1α (macrophage inflammatory protein-1α), MIP-1β (macrophage inflammatory protein-1β), TARC (Thymus and Activation Regulated Chemokine), and eotaxin was observed, indicative of PD activity. Peak serum concentrations of these cytokines and chemokines were reached within 1 day of administration and returned to pretreatment levels by Day 15. Soluble CD25 serum concentrations peaked around Day 4 after treatment and returned to pretreatment levels by Day 15.

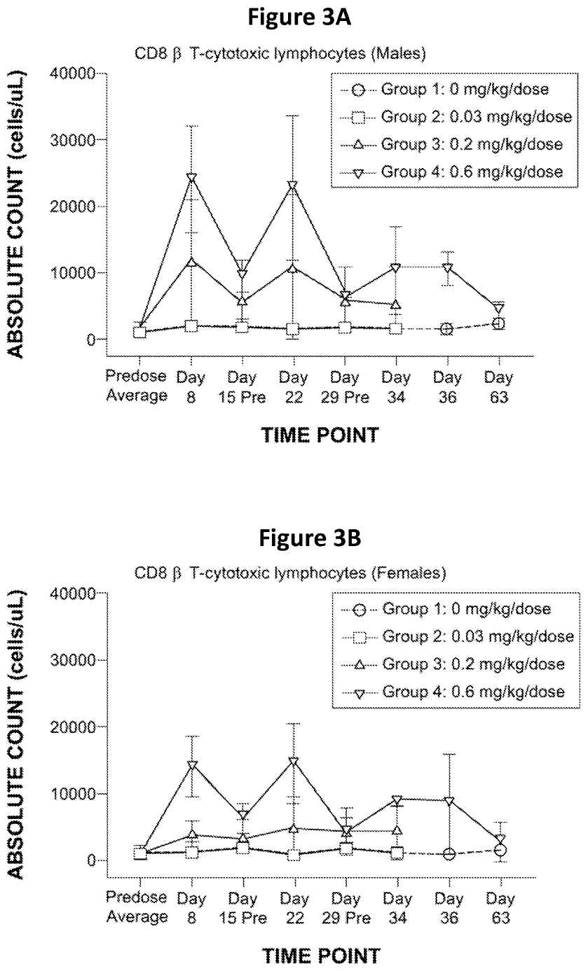

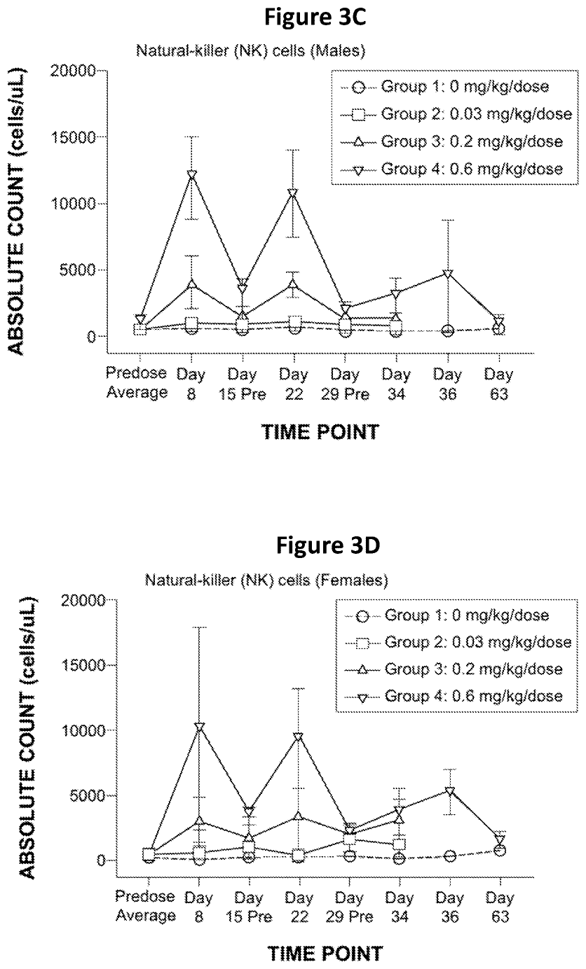

XENP24306+XENP32803 treatment expanded CD8⁻ T cell and NK-cell numbers in peripheral blood, validating the targeting of expected immune cell populations. Following an initial decrease in blood lymphocytes, likely due to margination, CD8⁺ T cells and NK cells exhibited dose-dependent expansion over pretreatment levels. Peak response in blood was achieved a week after dosing, and cell counts appeared to return close to pretreatment levels 2 weeks later. CD8⁺ memory T cell subsets, including central and effector memory, terminal effector, and stem cell memory cells were expanded, but naïve CD8⁺ T cells were not. CD4⁻ T cells, Tregs, B cells, and granulocytes showed either minimal expansion or were not responsive to XENP24306+XENP32803. A transient and dose-dependent increase in frequencies of Ki67 expression (cell proliferation marker) was also observed among these target cell populations consistent with expansion of absolute cell numbers. Repeat dosing of XENP24306+XENP32803 (0.03, 0.2, and 0.6 mg/kg, Q2W) showed transient elevations in cytokine and chemokine responses after each dose. Responses to XENP24306+XENP32803 were dose-dependent, and changes were reversible with cytokines, chemokines, and sCD25 levels. The repeat-dose toxicity study demonstrated that CD8⁺ T cell and NK-cell expansion (approximately 6-fold at mid dose and 14-17 fold at high dose) in peripheral blood was transient after each dose with lower peak counts observed following repeated XENP24306+XENP32803 treatment (FIG. 3). Peripheral CD8⁺ T cell and NK-cell numbers returned to pretreatment levels after a 4-week recovery period.

Subjects with multiple myeloma or leukemia may be treated with stem cell transplantation but complications including graft-versus-host-disease (GVHD) can arise when the donor's T cells (graft) attack the subject's healthy cells (host). The ability of XENP24306+XENP32803 to enhance leukocyte proliferation and effector activity was tested in a repeat dose study in a mouse graft-versus-host-disease (GVHD) model. XENP24306+XENP32803 (at four dose levels of 0.01, 0.03, 0.1, or 0.3 mg/kg, dosed on Days 0, 7, 14, and 21) was evaluated in non-obese diabetic/severe combined immunodeficient gamma (NSG) mice engrafted with human PBMCs, as a single agent. This study monitored an immune response against the mouse host that was measurable by clinical signs of GVHD (i.e., body weight loss and mortality), and immune monitoring assessments, such as elevations in peripheral human CD8⁺ T cell and NK-cell counts and serum IFNγ concentrations. Dose-dependent, GVHD-inducing activity was observed with significant body weight loss seen in mice treated with 0.3 mg/kg XENP24306+XENP32803, while significant elevations in CD8⁺ T cell and NK-cell counts and serum IFNγ concentrations were detected at lower doses. Time (Day 7, 14, 21) and dose-dependent increases in CD8⁺ T cell and NK-cell counts were observed. Expansion of CD4⁺ T cells was only observed on Day 14 at the two highest dose levels tested. The minimum pharmacologically active dose manifested by increased expansion of NK cells was 0.01 mg/kg, whereas higher doses were required to demonstrate significant enhancements of CD8⁺ T cells and serum IFNγ. Thus, XENP24306+XENP32803 promoted proliferation and effector enhancement of CD8⁺ T cells and NK cells that contributed to GVHD.

### Example 2: Pharmacokinetics and Drug Metabolism in Animals

A combination of XENP24306 (˜82%) and XENP32803 (˜18%) (“XENP24306+XENP32803”) binds to human and cynomolgus monkey IL-2/IL-15βγ heterodimeric receptor complex with comparable affinities and is active on both human and cynomolgus monkey CD8⁺ T cells and NK cells. Therefore, pharmacokinetics (PK) of XENP24306+XENP32803 were investigated in cynomolgus monkeys to support dose selection for Good Laboratory Practice (GLP) toxicity studies and to support selection of dose and dose regimen in the first-in-human (FIH) study. To support GLP toxicity studies, an electrochemiluminescent assay was developed and validated to quantify XENP24306+XENP32803 in cynomolgus monkey serum samples. Goat anti-human IL-15Rα antibody was used as capture, while mouse anti-human/primate IL-15 biotinylated antibody and sulfo-tagged streptavidin were used as primary and secondary detection reagents. The lower limit of quantification (LLOQ) was 30.0 ng/mL.

A time-resolved fluorescence method was developed to quantify XENP24306+XENP32803 concentrations in non-GLP PK/PD studies in cynomolgus monkey serum samples. The LLOQ in this assay was 1.4 ng/mL.

### Single-Dose Pharmacokinetics in Cynomolgus Monkeys

A preliminary pilot study designed to assess efficacy and to help define the max tolerated dose for GLP study design was conducted. Single-dose pharmacokinetics of XENP24306+XENP32803 were characterized in two, independent PK/PD studies in cynomolgus monkeys at 3.0 mg/kg in males and at 0.6 mg/kg in females. XENP24306+XENP32803 demonstrated a multiphasic profile with a mean Clearance (CL) of 66.4 mL/day/kg and mean volume of distribution at steady state (Vₛₛ) of 107 mL/kg following a single, 3.0 mg/kg IV administration to male cynomolgus monkeys. Mean Cₘₐₓ and exposure (area under the concentration-time curve from Time 0 to infinity [AUC_{0-∞}]) was 69.6 μg/mL and 45.4 day·μg/mL, respectively. Following a single IV administration of 0.6 mg/kg XENP24306+XENP32803 to female cynomolgus monkeys, the mean Cmax was 11.9 μg/mL, exposure (AUC_{0-∞}) was 11.7 day·μg/mL, CL was 52.6 mL/day/kg, and Vₛₛ was 89.0 mL/kg. See Table 3.

TABLE 3
Summary (Mean ± SD) Pharmacokinetic parameters for
XENP24306 + XENP32803 following a single,
intravenous 3.0 mg/kg dose in male cynomolgus monkeys and
a single, intravenous 0.6 mg/kg dose in female cynomolgus monkeys

| | 3.0 mg/kg | 0.6 mg/kg |
|---|---|---|
| PK Parameter | (male; n = 3) | (female; n = 3) |
| Cmax(μg/mL) | 69.6 ± 5.03 | 11.9 ± 0.618 |
| AUC_{0-∞} (day · μg/mL) | 45.4^{a} | 11.7 ± 2.1 |
| CL (mL/day/kg) | 66.4^{a} | 52.6 ± 8.81 |
| Vₛₛ(mL/kg) | 107^{a} | 89.0 ± 4.58 |

^{a}Mean of 2 animals, therefore no SD reported. The 3.0 mg/kg dose was not well tolerated.

### Repeat-Dose Pharmacokinetics in Cynomolgus Monkeys

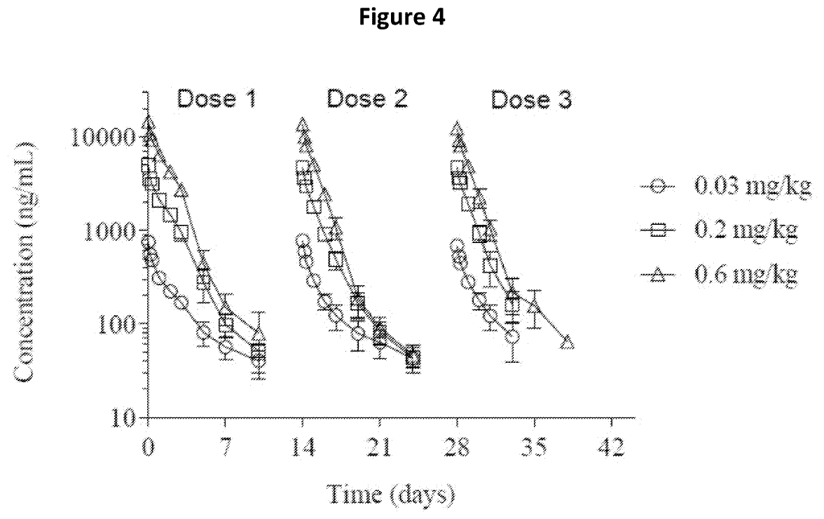

The toxicokinetics (TK) of XENP24306+XENP32803 were characterized in a 5-week, GLP, repeat-dose, toxicity study in cynomolgus monkeys. Three dose levels (0.03, 0.2, and 0.6 mg/kg XENP24306+XENP32803) were given at 14-day intervals for a total of 3 doses. Systemic exposure was confirmed in all animals and there were no sex differences observed in NENP24306+XENP32803 exposure in cynomolgus monkeys (FIG. 4). The Cₘₐₓ was dose-proportional after the first dose. There was a slight trend for decreasing Cₘₐₓ with repeated dosing; however, the ranges (mean SD) were overlapping for Cmax after the first, second, and third doses. The AUC₀₋₁₄ was slightly less than dose-proportional after the first dose. In addition to this, exposure (AUC) decreased with repeated XENP24306+XENP32803 dosing, particularly at the 0.2 mg/kg dose (from 7.74 to 5.96 day·μg g/mL, 22% decrease) and the 0.6 mg/kg dose (from 21.1 to 14.9 day·μg/mL, 30% decrease; Table 4). This decrease in systemic exposure (AUC) upon repeated dosing might be attributed to an increase in TMDD as a result of increased target-cell population. The XENP24306+XENP32803 CL after the first dose ranged from 18 to 28 mL/day/kg, and the Vₛₛ was in the range of 52 to 86 mL/kg. The higher-than-normal IgG clearances (<10 mL/day/kg for a typical IgG) of XENP24306+XENP32803 observed in these studies were likely a consequence of TMDD. Time-varying, non-linear PK behavior was observed for XENP24306+XENP32803 across dose levels as indicated by increased CL with increased dose after the first dose and a further less-than-dose-proportional increase in AUC₀₋₁₄ after repeat dosing. A similar PK behavior is expected for XENP24306+XENP32803 in humans. Increased target-cell population in response to XENP24306+XENP32803 dosing was expected to increase the TMDD effect leading to time varying pharmacokinetics, as observed in this study. No accumulation was observed following repeated administration as indicated by decreasing AUC values, with an AUC ratio of 0.704- to 0.991-fold between the first and second doses (Table 4).

TABLE 4
Group mean (±SD) toxicokinetic parameters (males and
females combined) for XENP24306 + XENP32803 in cynomolgus
monkeys following Q2W (every 2 weeks) intravenous dosing.

| | Group 2 | Group 3 | Group 4 |
|---|---|---|---|
| Toxicokinetic Parameter | (0.03 mg/kg) | (0.2 mg/kg) | (0.6 mg/kg) |
| Cₘₐₓ, first dose (μg/mL) | 0.750 ± 0.0410 | 5.03 ± 0.851 | 14.7 ± 1.73 |
| Cₘₐₓ, second dose (μg/mL) | 0.776 ± 0.0415 | 4.73 ± 0.455 | 13.6 ± 1.88 |
| Cₘₐₓ, third dose (μg/mL) | 0.687 ± 0.0510 | 4.75 ± 0.555 | 12.4 ± 1.58 |
| AUC₀₋₁₄, first dose | 1.56 ± 0.148 | 7.74 ± 0.960 | 21.1 ± 1.21 |
| (day · μg/mL) | | | |
| AUC₀₋₁₄, second dose | 1.55 ± 0.247 | 5.96 ± 0.489 | 14.9 ± 1.36 |
| (day · μg /mL) | | | |
| CL, first dose (mL/day/kg) | 17.9 ± 2.22 | 26.0 ± 3.09 | 28.4 ± 1.61 |
| Vₛₛ, first dose (mL/kg) | 86.2 ± 6.31 | 56.1 ± 5.72 | 52.3 ± 6.98 |

### Example 3: Pharmacodynamic Effects

### Effect on Cytokines, Chemokines and Soluble CD25

Cytokines were assessed following single-dose 0.6 or 3.0 mg/kg of a combination of IL15/IL15Rα heterodimeric proteins (XENP24306 (˜82%) and XENP32803 (˜18%) (“XENP24306+XENP32803”)) in two, independent, cynomolgus monkey PK/PD studies). At both the 0.6 mg/kg and 3.0 mg/kg XENP24306+XENP32803 dose, elevations of serum markers as well as cytokines and chemokines peaked within 8 to 16 hours following dosing and generally returned to pretreatment levels by day 15. Serum markers that were elevated following XENP24306+XENP32803 treatment included eotaxin, eotaxin-3, IL-8, IP-10, MCP-1, MCP-4, MDC, MIP-1a, MIP-1s, and TARC. Increased expression of these cytokines and chemokines may further contribute to the lymphocyte expansions induced by XENP24306+XENP32803.

In two, independent, PK/PD studies, sCD25/IL-2Ra was assessed following a single dose of 0.6 or 3.0 mg/kg XENP24306+XENP32803. At both the 0.6 mg/kg and 3.0 mg/kg XENP24306+XENP32803 dose groups, the pattern for sCD25 showed gradual increases 3 to 4 days following dosing, which aligned with CD25 expression on T cells.

### Effect on Lymphocytes

After a single dose of 0.6 mg/kg or 3.0 mg/kg XENP24306+XENP32803, lymphocytes were mildly-to-moderately decreased until 3 days following dosing. This was followed by a variable, dose-dependent, moderate-to-marked increase that peaked 7 to 9 days after dosing. Lymphocytes were subsequently recovered or partially recovered towards pretreatment levels by end of study. Monocytes tended to mirror lymphocytes, but to a much lesser degree. Blood smear examination performed on the 0.6 mg/kg-dose animals noted that many of the lymphocytes were atypical/reactive.

### Mononuclear Cell Infiltration

Following single-dose 0.6 mg/kg XENP24306+XENP32803, minimal-to-mild mononuclear cell infiltration was observed in the sinusoids of the liver. At single-dose 3.0 mg/kg XENP24306+XENP32803, mononuclear-cell infiltrates were noted in the liver, kidneys, lung, jejunum, urinary bladder, and skin.

### Example 4: Repeat-Dose Toxicity

Two, repeat-dose, GLP studies were conducted: (1) a 5-week toxicity study with a 4-week recovery period described in this Example and (2) a dedicated cardiovascular safety pharmacology study described in Example 5.

The 5-week, repeat-dose, GLP toxicity study was conducted in male and female cynomolgus monkeys to evaluate toxicity, pharmacology, and TK of a combination of IL15/IL15Rα heterodimeric proteins (XENP24306 (˜82%) and XENP32803 (˜18%) (“XENP24306+XENP32803”)). Animals either received vehicle (control group) or were dosed with 0.03, 0.2, or 0.6 mg/kg XENP24306+XENP32803 via IV bolus on Days 1, 15, and 29, and underwent necropsy on Day 34 (main study cohort) or Day 64 (recovery cohort; control and 0.6 mg/kg XENP24306). The 30-day recovery period was designed to assess reversibility or persistence of any XENP24306+XENP32803-related effects.

Assessment of toxicity was based on clinical observations, body weight, qualitative food evaluation, ophthalmology, ECG, clinical pathology parameters (hematology, coagulation, clinical chemistry, urinalysis, and urine chemistry), bioanalytical and TK parameters, ADA, cytokines, flow cytometry analyses, gross necropsy findings, organ weights, and histopathologic examinations.

TK analysis confirmed systemic exposure of XENP24306+XENP32803 at all dose levels tested. There were no differences in exposure between sexes. The Cₘₐₓ was dose proportional after the first dose. The AUC₀₋₁₄ after the first dose increased with dose, but was slightly less than dose proportional, and exposure (AUC) decreased upon repeated dosing. XENP24306+XENP32803 appeared to have non-linear kinetics in cynomolgus monkeys due to TMDD at the dose levels tested (Example 2).

All findings in the repeat-dose GLP toxicity study were consistent with the expected pharmacologic response of T cell and NK-cell expansion and activation with an associated pro-inflammatory response. The NOAEL defined from the dedicated repeat-dose, GLP toxicity study was determined to be 0.03 mg/kg XENP24306+XENP32803. Corresponding safety margins of the proposed XENP24306+XENP32803 FIH dose of 0.01 mg/kg IV Q2W to the NOAEL are described in Example 5.

### Example 5: Safety Pharmacology

A single, dedicated, GLP safety pharmacology study was performed in telemetry-instrumented male cynomolgus monkeys (four per group, including a vehicle control group) to assess the potential effects of a combination of IL15/IL15Rα heterodimeric proteins (XENP24306 (˜82%) and XENP32803 (˜18%) (“XENP24306+XENP32803”)) on the cardiovascular system. XENP24306+XENP32803 was administered at 0.03, 0.2, and 0.6 mg/kg (same doses as in the GLP toxicity study) by IV bolus injection on Days 1 and 15, and animals returned to the colony on Day 23. The following parameters and end points were evaluated: clinical signs, food consumption (qualitative evaluation), body weight, cardiovascular evaluation (systolic, diastolic, and MAP, heart rate, and ECG (including qualitative evaluation, and measurements of the RR-, PR-, QRS-, and QT-intervals and derived heart rate-corrected QT [QTca] interval), body temperature, serum albumin concentrations, and XENP24306+XENP32803 exposure and ADA incidence.

XENP24306+XENP32803 was clinically well tolerated at all doses (0.03, 0.2, and 0.6 mg/kg) with all animals surviving the study period and no veterinary intervention required. No adverse clinical signs, test article-related changes in food consumption, body weight changes, or ECG abnormalities were observed at any dose. ECGs were considered qualitatively normal for the cynomolgus monkey with no treatment-related changes in PR-, QRS-, or QTca-intervals.

Systemic exposure of XENP24306+XENP32803 was demonstrated at all dose levels. No treatment-related changes in body weight or qualitative food consumption occurred during the study.

Based on the totality of findings from GLP studies in cynomolgus monkeys, the no-observed-adverse-effect level (NOAEL) dose was considered to be 0.03 mg/kg XENP24306+XENP32803. Due to the immune agonist properties of XENP24306+XENP32803, determination of the FIH dose was based on a minimum anticipated biological effect level (MABEL) approach. A dose of 0.01 mg/kg XENP24306+XENP32803, IV, as a single agent is proposed as the FIH dose for XENP24306+XENP32803. This FIH dose is based on EC₂₀ (0.23 μg/mL; geometric mean of 20 donors) and was derived using in vitro NK-cell (CD3⁻CD56⁺) proliferation (percent of cells that express Ki67) in human PBMCs, the most sensitive in vitro assay with XENP24306+XENP32803. See FIG. 1. The recommended FIH dose of 0.01 mg/kg XENP24306+XENP32803 is anticipated to be safe and is expected to provide minimal biological effect with minimal risk for treatment-mediated reactions in humans. Cₘₐₓ of XENP24306+XENP32803 administered IV in humans at the recommended FIH dose (i.e., at 0.01 mg/kg) is not expected to exceed this EC₂O level. The starting dose of 0.01 mg/kg XENP24306+XENP32803 in humans has a three-fold safety margin to the NOAEL dose (0.03 mg/kg XENP24306+XENP32803, Q2W) in the 5-week, GLP toxicity study in cynomolgus monkeys. Cmax of XENP24306+XENP32803 administered IV in humans at 0.01 mg/kg XENP24306+XENP32803 is expected to be 3.3-fold below the observed Cmax (0.75±0.04 μg/mL; first dose) at the NOAEL dose in cynomolgus monkeys. See Table 5. Furthermore, AUC at 0.01 mg/kg XENP24306+XENP32803 in humans is expected to be 1.8-fold below the AUC observed at the NOAEL dose in cynomolgus monkeys (Table 5). In summary, the observed Cmax and AUC at the NOAEL of XENP24306+XENP32803 in a relevant nonclinical GLP toxicity model (cynomolgus monkeys) further support the MABEL-based starting dose of 0.01 mg/kg XENP24306+XENP32803 IV and provide sufficient safety margins (Table 5) for the study.

The dosing frequency of XENP24306+XENP32803 in humans is Q2W and is supported by the 5-week, cynomolgus monkey, GLP toxicity study, where XENP24306+XENP32803 was generally well tolerated when given Q2W with no significant, acute toxicities. Peak, peripheral PD response (target-cell expansion such as NK and CD8⁺ T cells) was achieved a week after dosing and these peripheral target cell counts were declining toward their baseline by end of 2 weeks, following XENP24306+XENP32803 administration. Furthermore, cytokines and chemokines indicative of PD activity peaked between 8 to 16 hours following dosing and returned to baseline within 14 days of dosing (See Example 3). Therefore, an initial dosing frequency of Q2W is considered appropriate in the monotherapy dose escalation study with XENP24306+XENP32803 with the dose-limiting toxicity observation period encompassing the first cycle of study treatment.

TABLE 5
Non-clinical safety margin estimates for XENP24306 +
XENP32803 at proposed FIH dose: dose, AUC, and
Cₘₐₓ based exposure multiples for the recommended
starting dose of XENP24306 + XENP32803 (0.01 mg/kg,
Q2W) versus NOAEL (0.03 mg/kg, Q2W) in the 5-Week,
GLP, Toxicity Study in Cynomolgus Monkeys

| | Cₘₐₓ | AUC | Dose |
|---|---|---|---|
| | (μg/mL) | (day · μg/mL)^{a} | (mg/kg) |
| Starting dose in human: 0.01 mg/kg | 0.23 | 0.86 | 0.01 |
| Anticipated values | | | |
| NOAEL in cynomolgus monkey: | 0.75 | 1.56 | 0.03 |
| 0.03 mg/kg | | | |
| Observed values | | | |
| Safety margins | 3.3x | 1.8x | 3x |

AUC = area under the concentration-time curve;
Cmax = maximum observed serum concentration;
GLP = Good Laboratory Practice;
IV = intravenous;
NOAEL = no-observed-adverse-effect level;
Q2W = every 2 weeks.
^{a}AUCₕᵤₘₐₙ is predicted AUC₀₋₁₄ (i.e., dose/scaled human clearance) and AUC_{cyno} is observed AUC₀₋₁₄ after the first dose at NOAEL (0.03 mg/kg) in the 5-week, GLP toxicity study in cynomolgus monkeys.
Scaled human clearance = 11.6 mL/day/kg.

### Example 6: Combination Therapy, Open-Label, Multicenter, Global, Dose-Escalation Study of IL15/IL15Rα in Combination with Daratumumab

A combination therapy, open-label, multicenter, global, dose-escalation study to evaluate the safety, tolerability, and pharmacokinetics of IL15/IL15Rα heterodimeric proteins (XENP24306 (˜82%) and XENP32803 (˜18%) (“XENP24306+XENP32803”)) in combination with the anti-CD38 antibody daratumumab will be conducted in subjects who have received prior treatments (e.g., an immunomodulatory drug (IMiD), a proteasome inhibitor, or an anti-CD38 monoclonal antibody).

The study consists of a screening period of up to 28 days, a treatment period, and a minimum follow-up period of 90 days after treatment.

Subjects will be enrolled in two stages: a dose-escalation stage and an expansion stage.

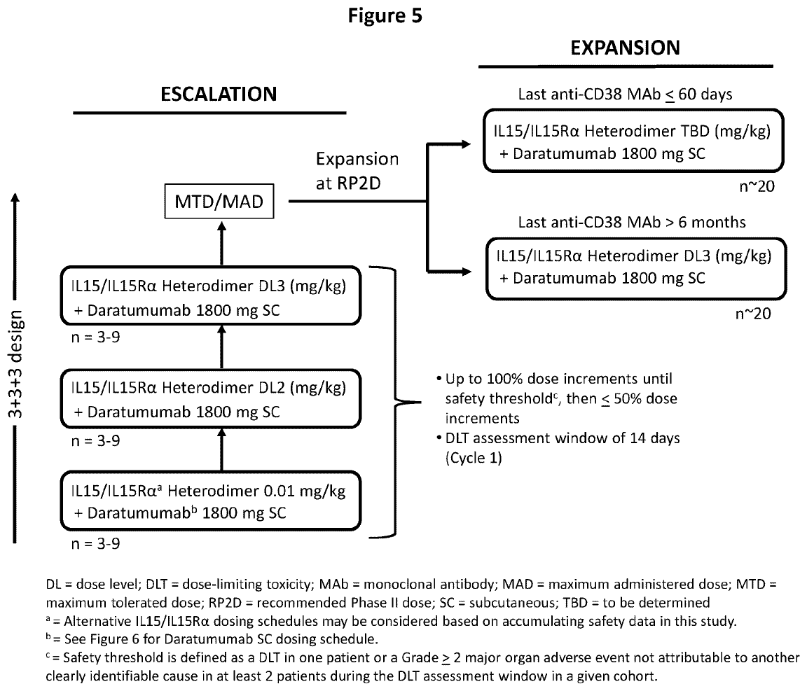

Cohorts of 3-9 subjects with a blood cancer (e.g., relapsed or refractory multiple myeloma) will be enrolled in the dose-escalation stage for the combination therapy portion of the study. XENP24306+XENP32803 at escalating doses will be administered by IV infusion and 1800 mg daratumumab will be administered subcutaneously following a 3+3+3 design (FIG. 5) to determine the maximum tolerated dose (MTD) or maximum administered dose (MAD) for XENP24306+XENP32803 in combination with daratumumab.

A provisional XENP24306+XENP32803 recommended Phase II dose (RP2D) at or below the MTD and MAD will be established in the dose-escalation stage. After establishing the RP2D, additional subjects will be enrolled in the expansion stage and treated at the RP2D. A total of approximately 60 subjects will enroll in the study at different global investigative sites.

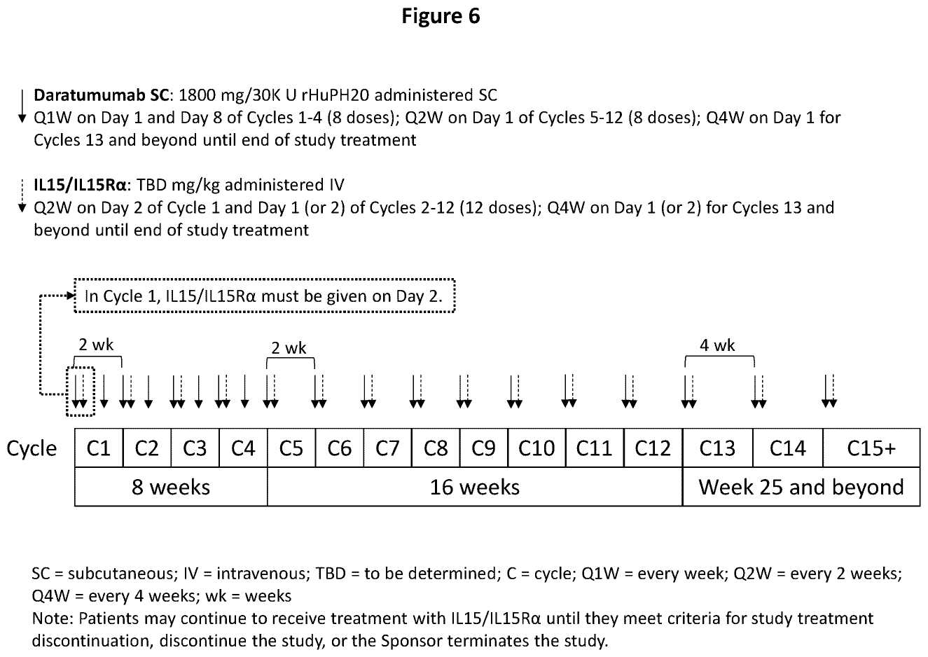

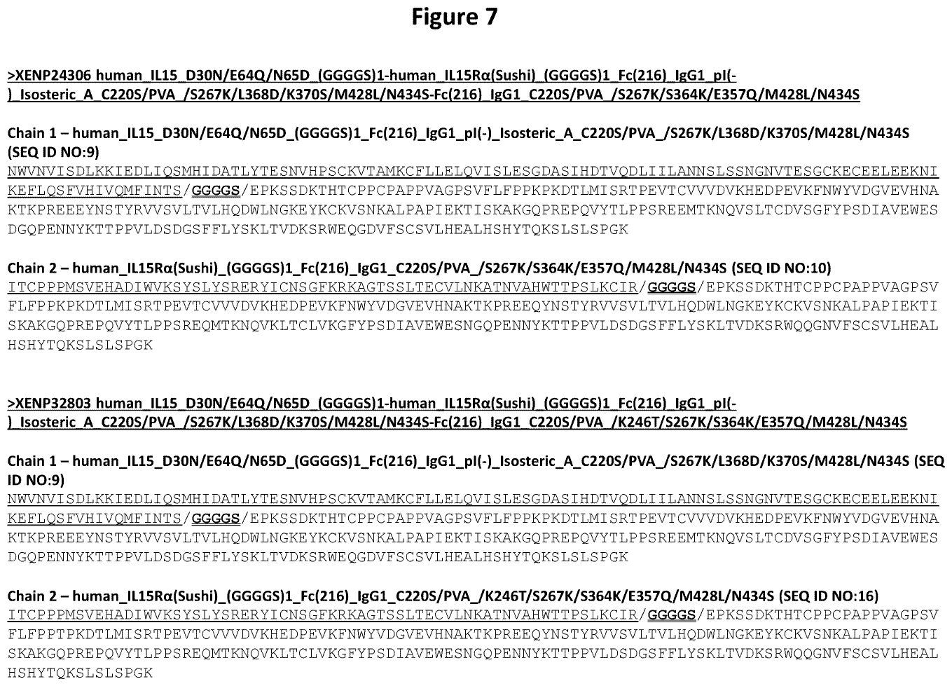

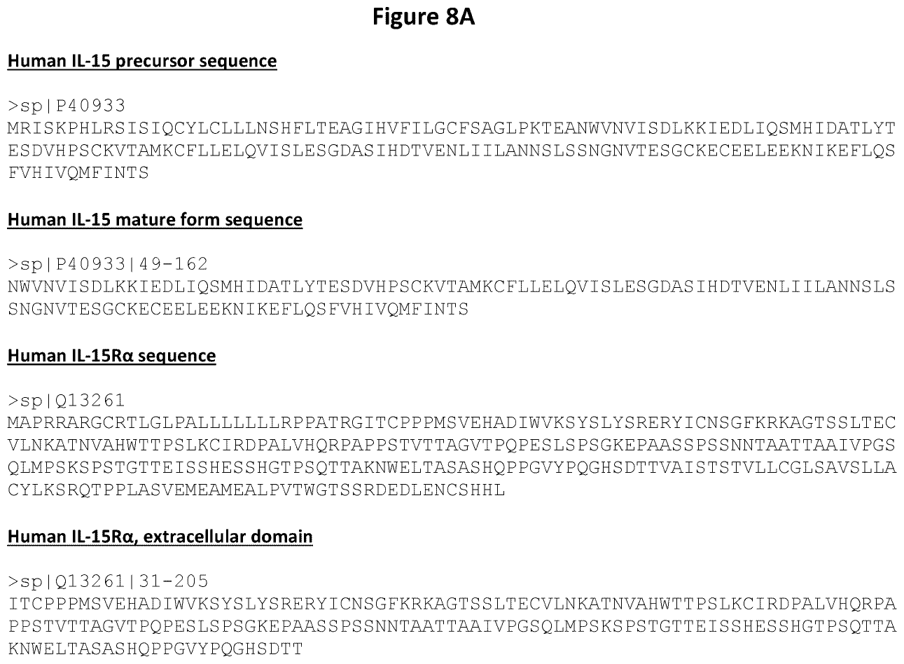

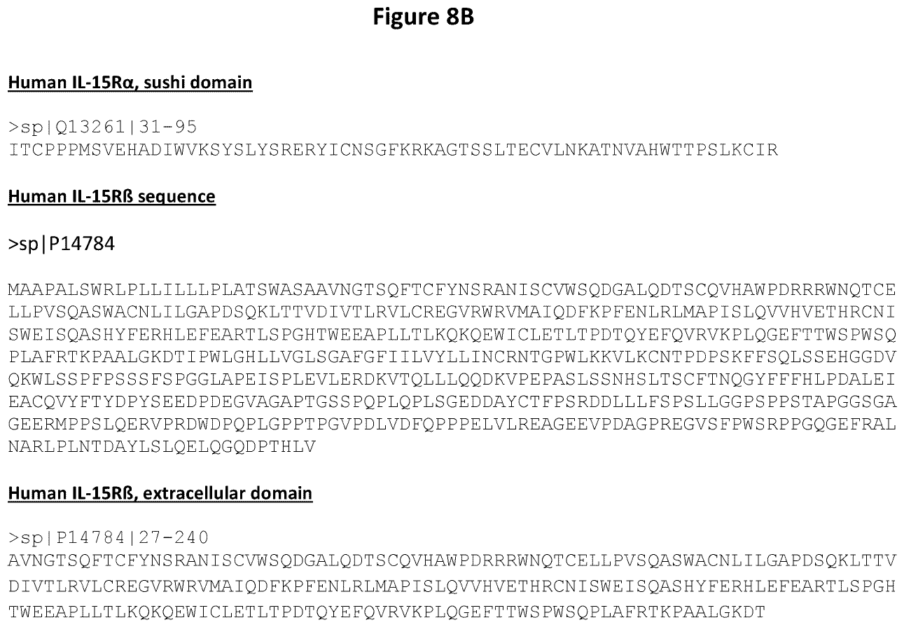

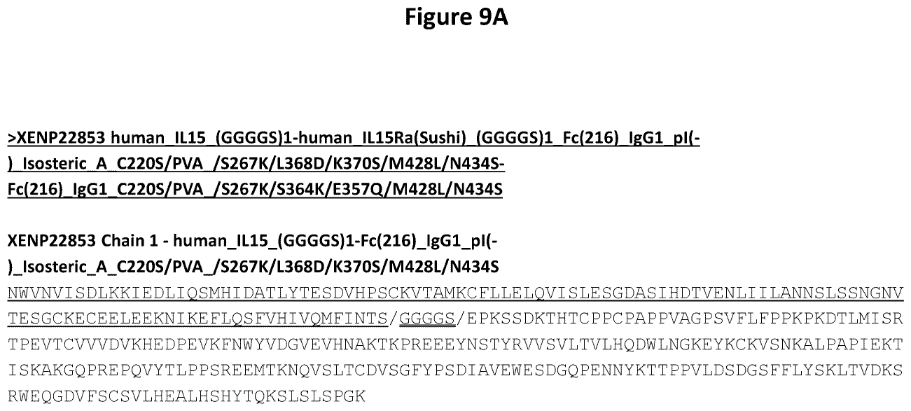

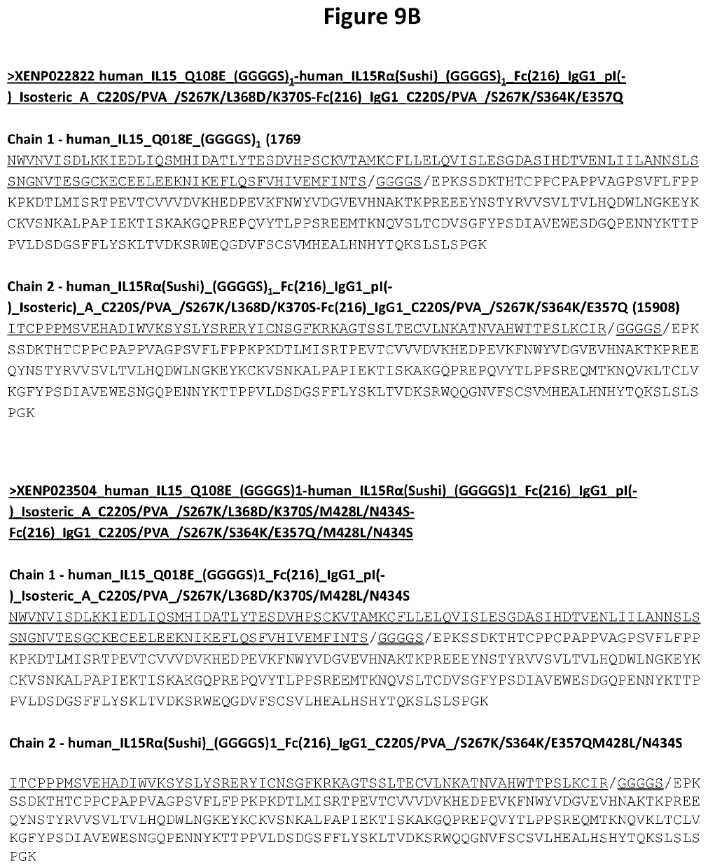

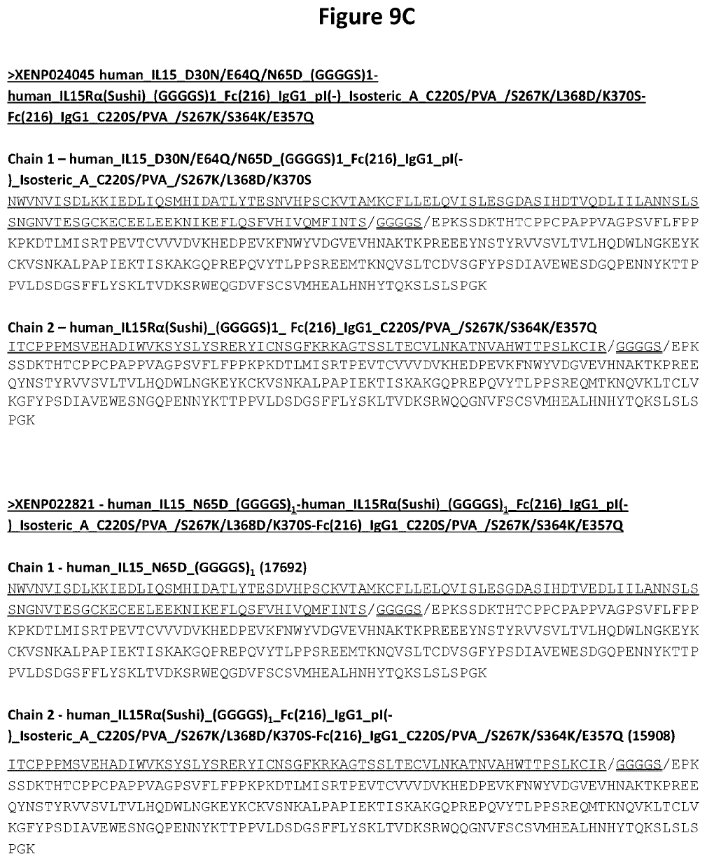

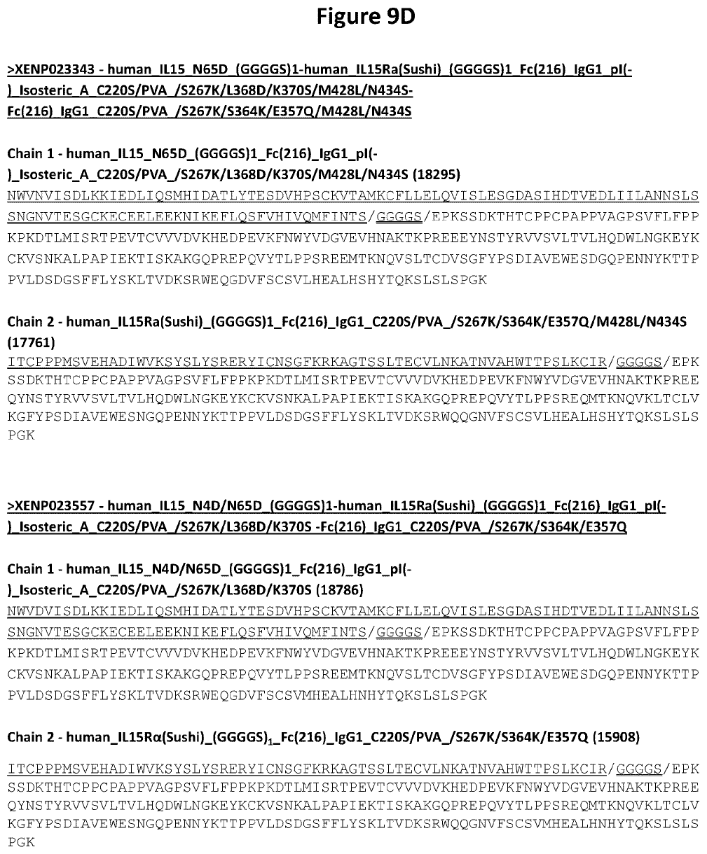

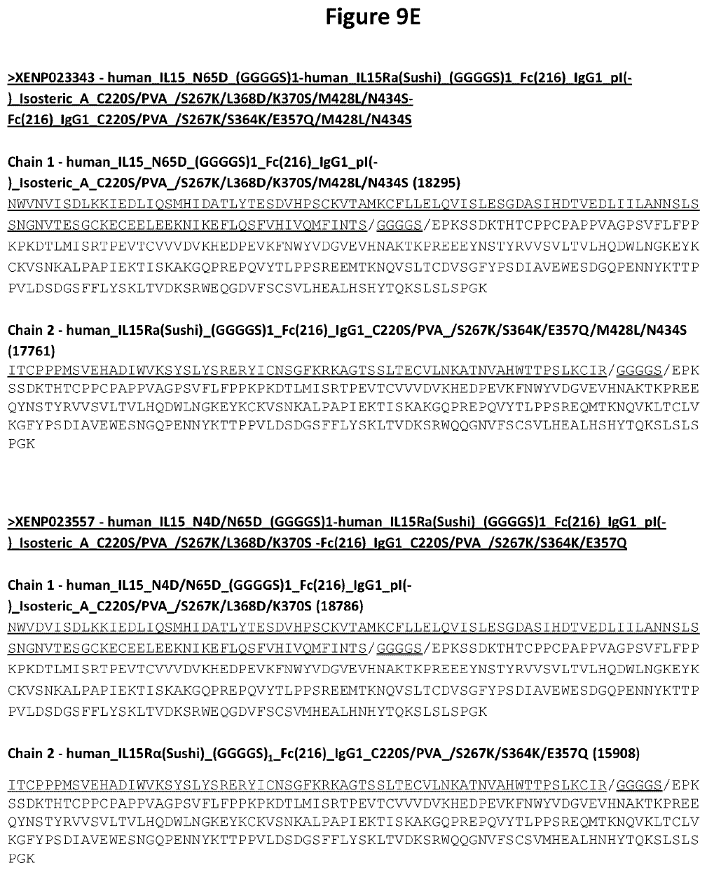

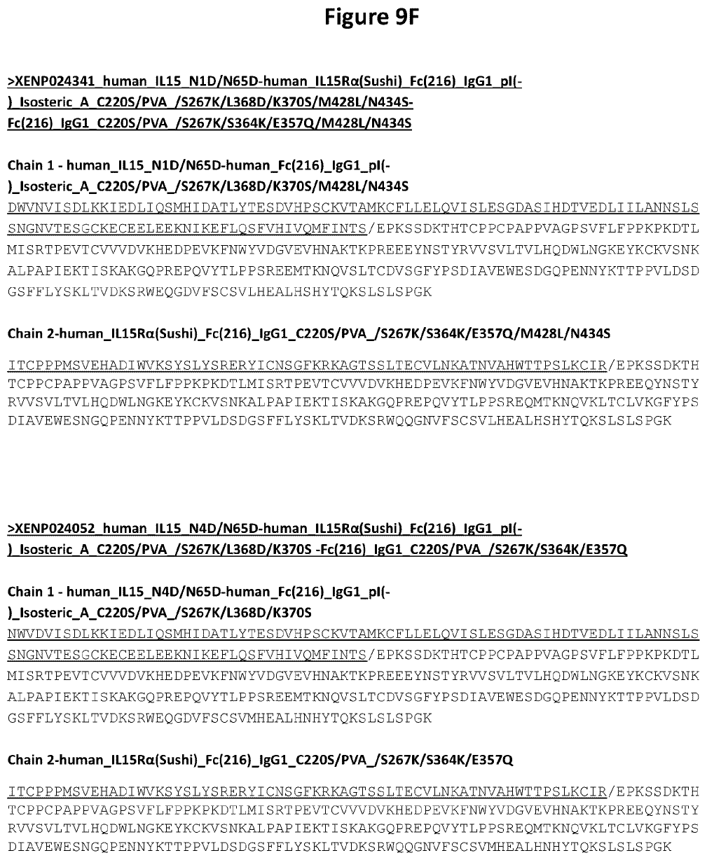

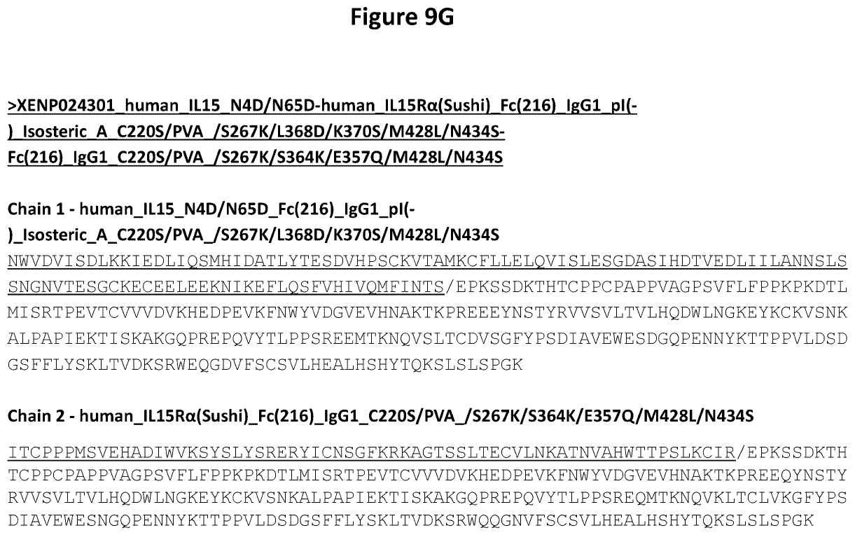

Following confirmation of eligibility, subjects will receive XENP24306+XENP32803 in combination with daratumumab. XENP24306+XENP32803 will be administered by IV infusion (starting at 0.01 mg/kg) every 2 weeks (Q2W) for Cycles 1-12, then every 4 weeks (Q4W) starting at Cycle 13 and beyond. Daratumumab will be administered subcutaneously (SC) every week (Q1W) for Cycles 1-4, Q2W for Cycles 5-12, then Q4W starting at Cycle 13 and beyond per the daratumumab SC monotherapy prescribing information (see, e.g., Darzalex SmPC). Study treatment cycles last 2 weeks for Cycles 1-12 and 4 weeks starting with Cycle 13 and beyond (FIG. 6). Subjects will be evaluated weekly by physical examination and routine hematologic and metabolic laboratory monitoring for the first four cycles of the combination treatment and less frequently thereafter.

All adverse events will be reported until 30 days after the final dose of study treatment or until initiation of new systemic anti-cancer therapy, whichever comes first. Serious adverse events and adverse events of special interest will continue to be reported until 90 days after the final dose of study treatment or until initiation of new systemic anti-cancer therapy, whichever occurs first. Adverse events will be graded according to NCI CTCAE v5.0.

Subjects will undergo disease assessments at screening (baseline) and at regular intervals during the study, which will be measured according to IMWG Uniform Response Criteria. Subjects may continue treatment with XENP24306+XENP32803 and daratumumab until disease progression as determined by the investigator according to IMWG Uniform Response Criteria, unacceptable toxicity, start of a new anti-cancer therapy, or withdrawal from the study.

Subjects who permanently discontinue XENP24306+XENP32803 and daratumumab will return to the clinic for a treatment discontinuation visit within 30 days after the final dose of study treatment. The visit at which response assessment shows progressive disease may be used as the treatment discontinuation visit.

To characterize the safety, tolerability, pharmacokinetics, activity of XENP24306+XENP32803 in combination with daratumumab, blood samples will be taken at various timepoints before and after dosing.

The safety objective for this study is to evaluate the safety tolerability of XENP24306+XENP32803 in combination with daratumumab on the basis of the following endpoints:
Incidence and severity of adverse events, with severity determined according to National Cancer Institute Common Terminology Criteria for Adverse Events, Version 5.0 (NCI CTCAE v5.0); with the exception of cytokine-release syndrome (CRS), which will be graded according to the American Society for Transplantation and Cellular Therapy (ASTCT)
Change from baseline in targeted vital signs
Change from baseline in targeted clinical laboratory test results
Change from baseline in ECG parameters

The pharmacokinetic (PK) objective for this study is to characterize the PK profile of XENP24306+XENP32803 in combination with daratumumab on the basis of the following endpoints:
Serum concentration of XENP24306+XENP32803
Serum concentration of daratumumab

The activity objective for this study is to make a preliminary assessment of the activity of XENP24306+XENP32803 when administered in combination with daratumumab, on the basis of the following endpoints:
Objective response rate (ORR), defined as the proportion of subjects with best overall response of stringent complete response (sCR), complete response (CR), very good partial response (VGPR), or partial response (PR) as determined according to International Myeloma Working Group (IlVIWG) criteria;
Duration of response (DOR), defined as the time from the first occurrence of a documented confirmed objective response (sCR, CR, VGPR, or PR) to disease progression or death from any cause during the study (defined as within 30 days after the final dose of study drug), as determined by the investigator according to IMWG criteria;
Progression-free survival (PFS), defined as the time from the first study treatment to the first occurrence of disease progression or death from any cause during the study (defined as within 30 days) after the final dose of study drug), whichever occurs first, as determined by the investigator according to IMWG criteria.

The immunogenicity objective for this study is to evaluate the immune response to XENP24306+XENP32803 in combination with daratumumab on the basis of the following endpoints:
Prevalence of XENP24306+XENP32803 anti-drug antibodies (ADAs) at baseline and incidence of XENP24306+XENP32803 ADAs during the study;
To characterize the immunogenicity of daratumumab when administered in combination with XENP24306+XENP32803 on the basis of the following endpoint: Prevalence of daratumumab ADAs at baseline and incidence of daratumumab ADAs during the study;
To evaluate potential effects of ADAs on the basis of the following endpoint: Relationship between ADA status and safety, PK, or activity endpoints.

The exploratory biomarker objective for this study is to identify and/or evaluate biomarkers that are predictive of response to XENP24306+XENP32803 and daratumumab (i.e., predictive biomarkers), are early surrogates of activity, are associated with progression to a more severe disease state (i.e., prognostic biomarkers), are associated with acquired resistance to XENP24306+XENP32803 and daratumumab, are associated with susceptibility to developing adverse events or can lead to improved adverse event monitoring or investigation (i.e., safety biomarkers), can provide evidence of XENP24306+XENP32803 and daratumumab activity (i.e., pharmacodynamic (PD) biomarkers), or can increase the knowledge and understanding of disease biology and drug safety, on the basis of the following endpoint:
Relationship between biomarkers in blood and bone marrow and safety, PK, activity, immunogenicity, or other biomarker endpoints.

### Example 7: Combination Therapy, Open-Label, Multicenter, Global, Dose-Escalation Study of XENP24306 in Combination with Daratumumab

A combination therapy, open-label, multicenter, global, dose-escalation study to evaluate the safety, tolerability, and pharmacokinetics of XENP24306 in combination with the anti-CD38 antibody daratumumab will be conducted in subjects who have received prior treatments (e.g., an immunomodulatory drug (IMiD), a proteasome inhibitor, or an anti-CD38 monoclonal antibody).

The study consists of a screening period of up to 28 days, a treatment period, and a minimum follow-up period of 90 days after treatment.

Subjects will be enrolled in two stages: a dose-escalation stage and an expansion stage.

Cohorts of 3-9 subjects with a blood cancer (e.g., relapsed or refractory multiple myeloma) will be enrolled in the dose-escalation stage for the combination therapy portion of the study. XENP24306 at escalating doses will be administered by IV infusion and 1800 mg daratumumab will be administered subcutaneously following a 3+3+3 design (FIG. 5) to determine the maximum tolerated dose (MTD) or maximum administered dose (MAD) for XENP24306 in combination with daratumumab.

A provisional XENP24306 recommended Phase II dose (RP2D) at or below the MTD and MAD will be established in the dose-escalation stage. After establishing the RP2D, additional subjects will be enrolled in the expansion stage and treated at the RP2D. A total of approximately 60 subjects will enroll in the study at different global investigative sites.

Following confirmation of eligibility, subjects will receive XENP24306 in combination with daratumumab. XENP24306 will be administered by IV infusion (starting at 0.01 mg/kg) every 2 weeks (Q2W) for Cycles 1-12, then every 4 weeks (Q4W) starting at Cycle 13 and beyond. Daratumumab will be administered subcutaneously (SC) every week (Q1W) for Cycles 1-4, Q2W for Cycles 5-12, then Q4W starting at Cycle 13 and beyond per the daratumumab SC monotherapy prescribing information (see, e.g., Darzalex SmPC). Study treatment cycles last 2 weeks for Cycles 1-12 and 4 weeks starting with Cycle 13 and beyond (FIG. 6). Subjects will be evaluated weekly by physical examination and routine hematologic and metabolic laboratory monitoring for the first four cycles of the combination treatment and less frequently thereafter.

All adverse events will be reported until 30 days after the final dose of study treatment or until initiation of new systemic anti-cancer therapy, whichever comes first. Serious adverse events and adverse events of special interest will continue to be reported until 90 days after the final dose of study treatment or until initiation of new systemic anti-cancer therapy, whichever occurs first. Adverse events will be graded according to NCI CTCAE v5.0.

Subjects will undergo disease assessments at screening (baseline) and at regular intervals during the study, which will be measured according to IMWG Uniform Response Criteria. Subjects may continue treatment with XENP24306 and daratumumab until disease progression as determined by the investigator according to IMWG Uniform Response Criteria, unacceptable toxicity, start of a new anti-cancer therapy, or withdrawal from the study.

Subjects who permanently discontinue XENP24306 and daratumumab will return to the clinic for a treatment discontinuation visit within 30 days after the final dose of study treatment. The visit at which response assessment shows progressive disease may be used as the treatment discontinuation visit.

To characterize the safety, tolerability, pharmacokinetics, activity of XENP24306 in combination with daratumumab, blood samples will be taken at various timepoints before and after dosing.

The safety objective for this study is to evaluate the safety tolerability of XENP24306 in combination with daratumumab on the basis of the following endpoints:
Incidence and severity of adverse events, with severity determined according to National Cancer Institute Common Terminology Criteria for Adverse Events, Version 5.0 (NCI CTCAE v5.0); with the exception of cytokine-release syndrome (CRS), which will be graded according to the American Society for Transplantation and Cellular Therapy (ASTCT)
Change from baseline in targeted vital signs
Change from baseline in targeted clinical laboratory test results
Change from baseline in ECG parameters

The pharmacokinetic (PK) objective for this study is to characterize the PK profile of XENP24306 in combination with daratumumab on the basis of the following endpoints:
Serum concentration of XENP24306
Serum concentration of daratumumab

The activity objective for this study is to make a preliminary assessment of the activity of XENP24306 when administered in combination with daratumumab, on the basis of the following endpoints:
Objective response rate (ORR), defined as the proportion of subjects with best overall response of stringent complete response (sCR), complete response (CR), very good partial response (VGPR), or partial response (PR) as determined according to International Myeloma Working Group (IMWG) criteria;
Duration of response (DOR), defined as the time from the first occurrence of a documented confirmed objective response (sCR, CR, VGPR, or PR) to disease progression or death from any cause during the study (defined as within 30 days after the final dose of study drug), as determined by the investigator according to IMWG criteria;
Progression-free survival (PFS), defined as the time from the first study treatment to the first occurrence of disease progression or death from any cause during the study (defined as within 30 days) after the final dose of study drug), whichever occurs first, as determined by the investigator according to IMWG criteria.

The immunogenicity objective for this study is to evaluate the immune response to XENP24306 in combination with daratumumab on the basis of the following endpoints:
Prevalence of XENP24306 anti-drug antibodies (ADAs) at baseline and incidence of XENP24306 ADAs during the study;
To characterize the immunogenicity of daratumumab when administered in combination with XENP24306 on the basis of the following endpoint: Prevalence of daratumumab ADAs at baseline and incidence of daratumumab ADAs during the study;
To evaluate potential effects of ADAs on the basis of the following endpoint: Relationship between ADA status and safety, PK, or activity endpoints.

The exploratory biomarker objective for this study is to identify and/or evaluate biomarkers that are predictive of response to XENP24306 and daratumumab (i.e., predictive biomarkers), are early surrogates of activity, are associated with progression to a more severe disease state (i.e., prognostic biomarkers), are associated with acquired resistance to XENP24306 and daratumumab, are associated with susceptibility to developing adverse events or can lead to improved adverse event monitoring or investigation (i.e., safety biomarkers), can provide evidence of XENP24306 and daratumumab activity (i.e., pharmacodynamic (PD) biomarkers), or can increase the knowledge and understanding of disease biology and drug safety, on the basis of the following endpoint:
Relationship between biomarkers in blood and bone marrow and safety, PK, activity, immunogenicity, or other biomarker endpoints.

### Example 8: Combination Therapy, Open-Label, Multicenter, Global, Dose-Escalation Study of XENP32803 in Combination with Daratumumab

A combination therapy, open-label, multicenter, global, dose-escalation study to evaluate the safety, tolerability, and pharmacokinetics of XENP32803 in combination with an anti-CD38 antibody such as daratumumab will be conducted in subjects who have received prior treatments (e.g., an immunomodulatory drug (IMiD), a proteasome inhibitor, and an anti-CD38 monoclonal antibody).

The study consists of a screening period of up to 28 days, a treatment period, and a minimum follow-up period of 90 days after treatment.

Subjects will be enrolled in two stages: a dose-escalation stage and an expansion stage.

Cohorts of 3-9 subjects with a blood cancer (e.g., relapsed or refractory multiple myeloma) will be enrolled in the dose-escalation stage for the combination therapy portion of the study. XENP32803 at escalating doses will be administered by IV infusion and 1800 mg daratumumab will be administered subcutaneously following a 3+3+3 design (FIG. 5) to determine the maximum tolerated dose (MTD) or maximum administered dose (MAD) for XENP32803 in combination with daratumumab.

A provisional XENP32803 recommended Phase II dose (RP2D) at or below the MTD and MAD will be established in the dose-escalation stage. After establishing the RP2D, additional subjects will be enrolled in the expansion stage and treated at the RP2D. A total of approximately 60 subjects will enroll in the study at different global investigative sites.

Following confirmation of eligibility, subjects will receive XENP32803 in combination with daratumumab. XENP32803 will be administered by IV infusion (starting at 0.01 mg/kg) every 2 weeks (Q2W) for Cycles 1-12, then every 4 weeks (Q4W) starting at Cycle 13 and beyond. Daratumumab will be administered subcutaneously (SC) every week (Q1W) for Cycles 1-4, Q2W for Cycles 5-12, then Q4W starting at Cycle 13 and beyond per the daratumumab SC monotherapy prescribing information (see, e.g., Darzalex SmPC). Study treatment cycles last 2 weeks for Cycles 1-12 and 4 weeks starting with Cycle 13 and beyond (FIG. 6). Subjects will be evaluated weekly by physical examination and routine hematologic and metabolic laboratory monitoring for the first four cycles of the combination treatment and less frequently thereafter.

All adverse events will be reported until 30 days after the final dose of study treatment or until initiation of new systemic anti-cancer therapy, whichever comes first. Serious adverse events and adverse events of special interest will continue to be reported until 90 days after the final dose of study treatment or until initiation of new systemic anti-cancer therapy, whichever occurs first. Adverse events will be graded according to NCI CTCAE v5.0.

Subjects will undergo disease assessments at screening (baseline) and at regular intervals during the study, which will be measured according to IMWG Uniform Response Criteria. Subjects may continue treatment with XENP32803 and daratumumab until disease progression as determined by the investigator according to IMWG Uniform Response Criteria, unacceptable toxicity, start of a new anti-cancer therapy, or withdrawal from the study.

Subjects who permanently discontinue XENP32803 and daratumumab will return to the clinic for a treatment discontinuation visit within 30 days after the final dose of study treatment. The visit at which response assessment shows progressive disease may be used as the treatment discontinuation visit.

To characterize the safety, tolerability, pharmacokinetics, activity of XENP32803 in combination with daratumumab, blood samples will be taken at various timepoints before and after dosing.

The safety objective for this study is to evaluate the safety tolerability of XENP32803 in combination with daratumumab on the basis of the following endpoints:
Incidence and severity of adverse events, with severity determined according to National Cancer Institute Common Terminology Criteria for Adverse Events, Version 5.0 (NCI CTCAE v5.0); with the exception of cytokine-release syndrome (CRS), which will be graded according to the American Society for Transplantation and Cellular Therapy (ASTCT)
Change from baseline in targeted vital signs
Change from baseline in targeted clinical laboratory test results
Change from baseline in ECG parameters

The pharmacokinetic (PK) objective for this study is to characterize the PK profile of XENP32803 in combination with daratumumab on the basis of the following endpoints:
Serum concentration of XENP32803
Serum concentration of daratumumab

The activity objective for this study is to make a preliminary assessment of the activity of XENP32803 when administered in combination with daratumumab, on the basis of the following endpoints:
Objective response rate (ORR), defined as the proportion of subjects with best overall response of stringent complete response (sCR), complete response (CR), very good partial response (VGPR), or partial response (PR) as determined according to International Myeloma Working Group (IMWG) criteria;
Duration of response (DOR), defined as the time from the first occurrence of a documented confirmed objective response (sCR, CR, VGPR, or PR) to disease progression or death from any cause during the study (defined as within 30 days after the final dose of study drug), as determined by the investigator according to IMWG criteria;
Progression-free survival (PFS), defined as the time from the first study treatment to the first occurrence of disease progression or death from any cause during the study (defined as within 30 days) after the final dose of study drug), whichever occurs first, as determined by the investigator according to IMWG criteria.

The immunogenicity objective for this study is to evaluate the immune response to XENP32803 in combination with daratumumab on the basis of the following endpoints:
Prevalence of XENP32803 anti-drug antibodies (ADAs) at baseline and incidence of XENP32803 ADAs during the study;
To characterize the immunogenicity of daratumumab when administered in combination with XENP32803 on the basis of the following endpoint: Prevalence of daratumumab ADAs at baseline and incidence of daratumumab ADAs during the study;
To evaluate potential effects of ADAs on the basis of the following endpoint: Relationship between ADA status and safety, PK, or activity endpoints.

The exploratory biomarker objective for this study is to identify and/or evaluate biomarkers that are predictive of response to XENP32803 and daratumumab (i.e., predictive biomarkers), are early surrogates of activity, are associated with progression to a more severe disease state (i.e., prognostic biomarkers), are associated with acquired resistance to XENP32803 and daratumumab, are associated with susceptibility to developing adverse events or can lead to improved adverse event monitoring or investigation (i.e., safety biomarkers), can provide evidence of XENP32803 and daratumumab activity (i.e., pharmacodynamic (PD) biomarkers), or can increase the knowledge and understanding of disease biology and drug safety, on the basis of the following endpoint:
Relationship between biomarkers in blood and bone marrow and safety, PK, activity, immunogenicity, or other biomarker endpoints.

1. A method of treating a blood cancer in a subject in need thereof, the method comprising administering to the subject a therapeutically effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.

2. A method for inducing the proliferation of CD8⁺ effector memory T cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.

3. A method for inducing the proliferation of NK cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.

4. A method for inducing the proliferation of CD8⁺ effector memory T cells and NK cells in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL-15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.

5. A method for inducing IFNγ production in a subject suffering from a blood cancer, the method comprising administering to the subject an effective amount of a heterodimeric protein, wherein the heterodimeric protein comprises (i) a first monomer comprising an IL-15 protein and a first Fc domain, wherein said IL15 protein is covalently attached to the N-terminus of said first Fc domain and (ii) a second monomer comprising a sushi domain of IL-15Rα protein and a second Fc domain, wherein said sushi domain of IL-15Rα protein is covalently attached to the N-terminus of said second Fc domain; and wherein said IL-15 protein comprises an N65D amino acid substitution and one or more amino acid substitutions selected from the group consisting of N4D, D30N, E64Q.

6. The method according to claim 1, wherein each of said first and second Fc domains comprises amino acid substitutions E233P, L234V, L235A, G236del, and S267K, according to EU numbering.

7. The method according to claim 1, wherein:
(i) said first Fc domain further comprises amino acid substitutions L368D and K370S and said second Fc domain further comprises amino acid substitutions S364K and E357Q, according to EU numbering; or
(ii) said first Fc domain further comprises amino acid substitutions S364K and E357Q and said second Fc domain further comprises amino acid substitutions L368D and K370S, according to EU numbering.

8. (canceled)

9. The method according to claim 1, wherein:
(i) said first Fc domain further comprises amino acid substitutions Q295E, N384D, Q418E and N421D, according to EU numbering;
(ii) said second Fc domain further comprises amino acid substitutions Q295E, N384D, Q418E and N421D, according to EU numbering: or
(iii) said first Fc domain further comprises amino acid substitutions Q295E, N384D, Q418E and N421D, according to EU numbering and said second Fc domain further comprises amino acid substitutions Q295E, N384D, Q418E and N421D, according to EU numbering.

10. (canceled)

11. The method according to claim 1, wherein said second Fc domain further comprises amino acid substitution K246T, according to EU numbering.

12. The method according to claim 1, wherein said IL-15 protein comprises amino acid substitutions D30N, E64Q and N65D.

13. The method according to claim 1, wherein said IL-15 protein comprises the amino acid sequence set forth in SEQ ID NO: 5.

14. The method according to claim 1, wherein said sushi domain of IL-15Rα protein comprises the amino acid sequence set forth in SEQ ID NO: 4.

15. The method according to claim 1, wherein:
(i) the IL-15 protein is covalently attached to the N-terminus of the first Fc domain via a first linker;
(ii) the IL-15Rα protein is covalently attached to the N-terminus of the second Fc domain via a second linker: or
(iii) the IL-15 protein is covalently attached to the N-terminus of the first Fc domain via a first linker and the IL-15Rα protein is covalently attached to the N-terminus of the second Fc domain via a second linker.

16.-17. (canceled)

18. The method according to claim 15, wherein the first linker and/or second linker is independently a variable length Gly-Ser linker selected from the group consisting of (Gly-Gly-Gly-Gly-Ser)n (SEQ ID NO: 39), (Ser-Ser-Ser-Ser-Gly)n (SEQ ID NO: 40), (Gly-Ser-Ser-Gly-Gly)n (SEQ ID NO: 41), and (Gly-Gly-Ser-Gly-Gly)n (SEQ ID NO: 42), where n is an integer between 1 and 5.

19.-42. (canceled)

43. The method according to claim 1, wherein said first monomer comprises the amino acid sequence set forth in SEQ ID NO: 9, and the second monomer comprises the amino acid sequence set forth in SEQ ID NO: 10 or SEQ ID NO: 16.

44.-49. (canceled)

50. The method according to claim 1, wherein said blood cancer is selected from the group consisting of leukemia, acute myeloid leukemia, adult acute lymphoblastic leukemia, chronic lymphocytic leukemia, lymphoma, non-Hodgkin's lymphoma, B-cell non-Hodgkin's lymphoma, and multiple myeloma.

51.-59. (canceled)

60. The method according to claim 1, wherein said heterodimeric protein or combination of heterodimeric proteins is administered at a dose selected from the group consisting of about 0.0025 mg/kg, about 0.005 mg/kg, about 0.01 mg/kg, about 0.015 mg/kg, about 0.02 mg/kg, about 0.025 mg/kg, about 0.03 mg/kg, about 0.04 mg/kg, about 0.05 mg/kg, about 0.06 mg/kg, about 0.08 mg/kg, about 0.1 mg/kg, about 0.12 mg/kg, about 0.16 mg/kg, about 0.2 mg/kg, about 0.24 mg/kg and about 0.32 mg/kg body weight.

61.-63. (canceled)

64. The method according to claim 1, wherein said method further comprises administering to the subject an anti-CD38 monoclonal antibody, optionally selected from the group consisting of daratumumab, isatuximab, mezagitamab, and felzartamab.

65.-68. (canceled)

69. The method according to claim 1, wherein said heterodimeric protein is administered at a frequency selected from the group consisting of Q1W, Q2W, Q3W, Q4W, Q5W and Q6W.

70.-72. (canceled)

73. The method according to claim 64, wherein said anti-CD38 monoclonal antibody is administered at a frequency selected from the group consisting of Q1W, Q2W, Q3W, Q4W, Q5W and Q6W.

74.-81. (canceled)


SEQUENCE LISTING (56 sequences)

SEQ ID NO: 1 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 4 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 5 - protein, 114 aa, synthetic construct
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 6 - protein, 231 aa, synthetic construct
EPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 7 - protein, 231 aa, synthetic construct
EPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 8 - protein, 231 aa, synthetic construct
EPKSSDKTHTCPPCPAPPVAGPSVFLFPPTPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 9 - protein, 350 aa, synthetic construct
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 10 - protein, 301 aa, synthetic construct
ITCPPPMSVEHADIWVKSYSLYSRERYICNSGFKRKAGTSSLTECVLNKATNVAHWTTPSLKCIRGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 11 - protein, 350 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 12 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 231 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKQPREPQVYTLPPSREEMTKNQVSLTCLVKGFYPSDIAVEWESNGQPENNYNTTPPMLDSDGSFFLYSKLTVDKSRWQQGNIFSCSVMHEALHNRFTQKSLSLSPGK

SEQ ID NO: 15 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 301 aa, synthetic construct
ITCPPPMSVEHADIWVKSYSLYSRERYICNSGFKRKAGTSSLTECVLNKATNVAHWTTPSLKCIRGGGGSEPKSSDKTHTCPPCPAPPVAGPSVFLFPPTPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 17 - protein, 350 aa, synthetic construct
NWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 18 - protein, 301 aa, synthetic construct
ITCPPPMSVEHADIWVKSYSLYSRERYICNSGFKRKAGTSSLTECVLNKATNVAHWTTPSLKCIRGGGGSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 350 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 20 - protein, 301 aa, synthetic construct; identical to SEQ ID NO: 18

SEQ ID NO: 21 - protein, 350 aa, synthetic construct
NWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 22 - protein, 301 aa, synthetic construct; identical to SEQ ID NO: 18

SEQ ID NO: 23 - protein, 350 aa, synthetic construct
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 24 - protein, 301 aa, synthetic construct; identical to SEQ ID NO: 18

SEQ ID NO: 25 - protein, 345 aa, synthetic construct
DWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 26 - protein, 296 aa, synthetic construct
ITCPPPMSVEHADIWVKSYSLYSRERYICNSGFKRKAGTSSLTECVLNKATNVAHWTTPSLKCIREPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 345 aa, synthetic construct
NWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 28 - protein, 296 aa, synthetic construct; identical to SEQ ID NO: 26

SEQ ID NO: 29 - protein, 350 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 30 - protein, 301 aa, synthetic construct; identical to SEQ ID NO: 10

SEQ ID NO: 31 - protein, 350 aa, synthetic construct
NWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 32 - protein, 301 aa, synthetic construct; identical to SEQ ID NO: 10

SEQ ID NO: 33 - protein, 350 aa, synthetic construct
NWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 34 - protein, 301 aa, synthetic construct; identical to SEQ ID NO: 10

SEQ ID NO: 35 - protein, 345 aa, synthetic construct
DWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 36 - protein, 296 aa, synthetic construct
ITCPPPMSVEHADIWVKSYSLYSRERYICNSGFKRKAGTSSLTECVLNKATNVAHWTTPSLKCIREPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 37 - protein, 345 aa, synthetic construct
NWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 38 - protein, 296 aa, synthetic construct; identical to SEQ ID NO: 36

SEQ ID NO: 39 - protein, 25 aa, synthetic construct
  SITE 1..25: note = "This sequence may encompass 1-5 'Gly Gly Gly Gly Ser'repeating units"
GGGGSGGGGSGGGGSGGGGSGGGGS

SEQ ID NO: 40 - protein, 25 aa, synthetic construct
  SITE 1..25: note = "This sequence may encompass 1-5 'Ser Ser Ser Ser Gly'repeating units"
SSSSGSSSSGSSSSGSSSSGSSSSG

SEQ ID NO: 41 - protein, 25 aa, synthetic construct
  SITE 1..25: note = "This sequence may encompass 1-5 'Gly Ser Ser Gly Gly'repeating units"
GSSGGGSSGGGSSGGGSSGGGSSGG

SEQ ID NO: 42 - protein, 25 aa, synthetic construct
  SITE 1..25: note = "This sequence may encompass 1-5 'Gly Gly Ser Gly Gly'repeating units"
GGSGGGGSGGGGSGGGGSGGGGSGG

SEQ ID NO: 43-44 - skipped (2 entries)

SEQ ID NO: 45 - protein, 6 aa, synthetic construct
TVAAPS

SEQ ID NO: 46 - skipped

SEQ ID NO: 47 - protein, 7 aa, synthetic construct
ASTSGPS

SEQ ID NO: 48 - protein, 18 aa, synthetic construct
KESGSVSSEQLAQFRSLD

SEQ ID NO: 49 - protein, 14 aa, synthetic construct
EGKSSGSGSESKST

SEQ ID NO: 50 - protein, 6 aa, synthetic construct
GGGGGG

SEQ ID NO: 51 - protein, 8 aa, synthetic construct
GGGGGGGG

SEQ ID NO: 52 - protein, 12 aa, synthetic construct
GSAGSAAGSGEF

SEQ ID NO: 53 - protein, 5 aa, synthetic construct
GGGGS

SEQ ID NO: 54 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 55 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 56 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT